(19) United States
(12) Patent Application Publication
(10) Pub. No.: US 2021/0037866 A1
(43) Pub. Date: Feb. 11, 2021

(54) EMULSIFIERS AND THE USES THEREOF

(71) Applicant: Phytoption LLC, West Lafayette, IN (US)

(72) Inventor: Jingmin Zhang, West Lafayette, IN (US)

(73) Assignee: Phytoption LLC, West Lafayette, IN (US)

(21) Appl. No.: 17/072,200

(22) Filed: Oct. 16, 2020

Related U.S. Application Data
(63) Continuation of application No. PCT/US2019/028411, filed on Apr. 19, 2019.
(60) Provisional application No. 62/659,950, filed on Apr. 19, 2018.
(60) Provisional application No. 62/698,193, filed on Jul. 15, 2018.
(60) Provisional application No. 62/731,960, filed on Sep. 16, 2018.

(51) Int. Cl. A23L 29/10; A21D 2/36; A21D 2/26; A23L 7/10; A23L 33/105

(52) U.S. Cl. CPC A23L 29/10; A21D 2/36; A23L 33/105; A23L 7/10; A21D 2/265

(57) ABSTRACT

The present application relates generally to emulsifiers and methods of production thereof, and specifically to emulsifiers prepared using renewable and/or agricultural products through a physical process. Flour of cereal grains, legumes, or other plant materials contains both protein and carbohydrate, in particular starch. The preparation procedure of flour emulsifier contains physical processing, including but not limited to specifically designed heating, milling, or the combinations of both. Emulsions of lipophilic materials are prepared using flour emulsifiers. The emulsions formed can be further dehydrated using spray drying, drum drying, freeze drying, vacuum drying, or other drying methods. The emulsions prepared using flour emulsifiers as well as their dehydrated products show enhanced stability against physical and chemical deteriorations as compared with those prepared using conventional emulsifiers, such as octenylsuccinate starch and gum arabic.

### CROSS REFERENCES

This application is a Continuation of International Application PCT/US2019/028411, filed Apr. 19, 2019 which claims the benefit of US Provisional Application 62/659,950, filed on Apr. 19, 2018, U.S. Provisional Application 62/698,193, filed on Jul. 15, 2018, and U.S. Provisional Application 62/731,960, filed on Sep. 16, 2018. The contents of which are expressly incorporated herein entirely.

### STATEMENT OF GOVERNMENT RIGHTS

This invention was made with government support under NSF 1556121, awarded by the National Science Foundation. The government has certain rights in the invention.

### TECHNICAL FIELD

The present application relates generally to flour emulsifiers and methods of production thereof, and specifically to emulsifiers prepared using renewable products of agriculture through a physical process. The flour emulsifiers can be used in both food and non-food applications. While the flour emulsifiers have a basic form of flour, they can be produced, processed, or formulated in other forms such as particulates, grits, granules, powders, dispersions, or suspensions that are suitable for specific applications. The flour emulsifiers primarily provide emulsification properties, and in certain circumstances and applications, they may also provide other functionalities such as bulking, stabilizing, texturizing, formulating, and protecting in food and non-food systems.

### BACKGROUND

This section introduces aspects that may help facilitate a better understanding of the disclosure. Accordingly, these statements are to be read in this light and are not to be understood as admissions about what is or is not prior art.

In general, the food industry selects food materials and ingredients according to a number of characteristics, such as cost (e.g. affordability), sustainability (e.g. sustained availability and consistent pricing), functionalities (e.g. desirable properties and performances), sensory qualities, and resources. With respect to the resource of food materials and ingredients, the consumers and the food industry increasingly prefer the use of natural (or non-synthetic) materials in processed foods, which is usually related to the health image, health benefit, and sustainability that natural materials may provide. On the other hand, synthetic or partially synthetic materials are usually considered as not consumer friendly, not environmentally friendly, and thus should be reduced, removed, and/or avoided.

Therefore, there is a great need in the food industry to use natural, non-synthetic food materials and ingredients, which has been driving the rapid growth of “clean label” foods. Clean label is a recent term that has been used to describe the absence of ingredients with unfamiliar or chemical-like names, which are mostly associated with synthetic (artificial) or partially synthetic ingredients labeled in the Ingredients List of processed foods. In addition to clean label and natural resource, the consumer and the food industry are also demanding low cost, superior functionalities, and the sustainability for food materials and ingredients, without compromising sensory qualities.

Associated with emulsions and encapsulation systems, a number of naturally occurring materials have been used as emulsifiers for food, such as gum arabic, saponin, caseinate, and lecithin. However, these emulsifiers have various disadvantages. For example, gum arabic has been frequently associated with issues related to sustainability and price fluctuations, a major concern for its users in the food industry. Lecithin materials are in general lipophilic, thus showing limited emulsification capability in oil-in-water emulsions. In addition, soy lecithin is usually associated with GMO (genetically modified organism) soybeans. Thus far, the use of saponin has been limited to the foaming agent in semi-frozen carbonated and non-carbonated beverages. Sodium caseinate is a dairy based material and its emulsification capability decreases in systems with high acidity. For all currently used natural emulsifiers, there has been always a great need to reduce the cost of use, which constitutes a large challenge to the food industry.

In addition to food systems, there are a number of other application systems that also demand the use of natural and/or sustainable ingredients or excipients, such as personal care, cosmetics, health care, pharmaceuticals, medicals, and agricultural applications. A number of industrial applications may also need natural and/or sustainable ingredients, such as detergents, industrial cleanings, coatings, paintings, fuel formulations, and constructions.

### SUMMARY

The present application relates generally to emulsifiers and methods of production thereof, and specifically to flour emulsifiers prepared using renewable agricultural products and through physical processes. Flour emulsifiers are prepared from cereal grains, legumes, or other plant materials that contain both protein and carbohydrate, in particular starch. Said plant material is a flour, meal, faction or whole grain of cereal grain, legume, tuber, root, stem, seed, nut of a plant, and the combination thereof, including but not limited to wheat, corn, rice, wild rice, barley, fonio, Job's tears, sorghum, millet, oats, rye, teff, triticale, buckwheat, tartary buckwheat, amaranth, quinoa, pitseed goosefoot, canihua, chia, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, arhar/toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taro, tuber, cassava (tapioca), water chestnut, arrowroot, sweet potato, Chinese yam, lotus root, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, and the combination thereof. The preparation process primarily contains physical processing, such as specifically designed combined heating and milling. Emulsions of lipophilic materials are prepared using flour emulsifiers. The emulsions formed can be further dehydrated using spray drying, drum drying, freeze drying, vacuum drying, or other drying methods. The emulsions prepared using flour emulsifiers and the dehydrated emulsions may show enhanced stability against physical and chemical deteriorations compared with the emulsions and the dehydrated emulsions prepared using conventional emulsifiers, such as octenylsuccinate starch (a type of modified starch), sodium caseinate, whey protein, pea protein, soy protein, whey protein, lecithin, and gum arabic.

In an emulsion-related encapsulation solid, the oil droplets are embedded in “wall materials” for desirable protection and release properties during the manufacturing, storage, and usages. Usually, the wall materials can be carbohydrate(s), protein(s), or their mixtures. Some wall materials may have multiple functionalities such as emulsifying and bulking, with gum arabic, octenylsuccinate starch (OSA-starch), and sodium caseinate as examples. Some wall materials can only be used as bulking agent, such as maltodextrin or corn syrup. For effective encapsulation, it is necessary to form an emulsion with desirable stability.

The flour emulsifiers described in this invention can be used to form emulsions that are used to prepare emulsions, encapsulation products, or products. In these applications, the flour emulsifiers can be used as an emulsifier, as a bulking agent, as a texturing agent, as a protective agent, as a wall material, or a combination thereof.

In general, when the particles size of an encapsulation product is at micrometer level, the word “microencapsulation” is usually used instead of encapsulations. In general, the flour emulsifiers described in this invention can be used in both encapsulation and microencapsulation products. As used herein, the word “encapsulation” is to indicate both encapsulation and microencapsulation.

An encapsulation composition that is prepared by dehydrating the flour emulsifier stabilized emulsion is disclosed.

In one illustrative embodiment, the present invention relates to a flour emulsifier composition,
(1) Wherein the flour emulsifier composition contains both protein component and carbohydrate component including starch, wherein the content of protein component is 1% to 85% (dry weight base) of the said flour emulsifier composition and the content of carbohydrate component including starch is 15% to 99% (dry weight base) of the said flour emulsifier composition;
(2) Wherein the crystallinity of the said starch in said flour emulsifier is less than 90% of the crystallinity of the starch in the original plant material;
(3) Wherein the flour emulsifier composition has emulsification property

In some illustrative embodiments, the present invention relates to a process of preparing flour emulsifiers comprising the steps of:
(1) Preparing a flour made from a plant material that contains both protein and starch, including but not limited to whole or fraction of cereal grains (rice, corn, wheat, barley, rye, oats, etc.), legume grains (bean, peas), and tubers (potato, sweet potato), nuts, seeds;
(2) Subjecting the flour through milling (2 min to 50 h) and heating (40° C. to 300° C., 2 min to 100 h) in one or a plurality of steps so that the starch crystallinity is reduced to less than 90% of the starch crystallinity of the original plant material, thus to generate a flour emulsifier with emulsification properties for the purpose of emulsifying lipophilic materials including but not limited to:
   a. Oil or fat from plant, animal, microbial, or petroleum sources, or their mixtures or mixtures with other materials,
   b. Colorants (such as carotenoid oleoresins), flavors, and or fragrant,
   c. Lipophilic vitamins (such as VA, VE), nutrients (such as EPA, DHA, co-enzyme Q10, lecithin), and antioxidants (such as lutein, curcumin, astaxanthin),
   d. Essential oils (such as orange oil, rosemary extract),
   e. Other lipophilic or hydrophobic compounds that are soluble in above lipophilic materials.

In some other illustrative embodiments, the present invention relates to a process of encapsulation using flour emulsifiers prepared according to the method disclosed herein.
(1) Conventional methods are used to prepare emulsion-based encapsulation solids
   a. Make oil-in-water emulsions
   b. Apply dehydration of emulsions using spray-drying, freeze-drying, drum drying, vacuum drying, etc.
   c. The encapsulation solids product has acceptable properties in one or more of the following:
      i. Oil yield
      ii. Oil loading capacity
      iii. Surface oil
      iv. Stability against lipid oxidation
      v. Hydration
      vi. Physical and chemical stability of emulsion formed through the hydration of encapsulates

In some illustrative embodiments, this invention relates to a process for preparing an emulsifier from a plant material selected from a flour, a meal, a fraction, or a whole grain of a cereal grain, a legume, a tuber, a root, a stem, or other plant materials, or a combination thereof, comprising one or a plurality of steps of milling and heating of said plant material selected from a flour, meal, fraction, or whole grain of a cereal grain, a legume, a tuber, a root, a stem, a nut, a seed, or other plant materials, or a combination thereof to afford an emulsifier, wherein said emulsifier comprises at least a protein component and a carbohydrate component including starch, and wherein the crystallinity of starch is less than 90% of the crystallinity of starch in the original plant material.

In some illustrative embodiments, this invention relates to a process for preparing an emulsifier from a plant material selected from a flour, a meal, a fraction, or a whole grain of cereal grains, legumes, a tuber, a root, a stem, a nut, a seed, or other plant materials or a combination thereof, comprising one or a plurality of steps of milling and heating of said plant material selected from a flour, meal, fraction, or whole grain of cereal grains, legumes, a tuber, a root, a stem, a nut, a seed, or other plant materials or a combination thereof to afford an emulsifier,
(1) Wherein each said milling process provides a powder input of not less than 0.05 kilowatts (kw) per kilogram (kg) of the said plant material;
(2) wherein each said milling process lasts about 2 minutes to about 50 hours;
(3) wherein each said heating process has a temperature from about 40° C. to about 300° C. for a period of about 2 minutes to about 100 hours; and
(4) wherein said emulsifier contains about 1% to about 85% protein and about 15% to 99% carbohydrate including starch with a starch crystallinity less than 90% of the starch crystallinity of the original plant material.

In some illustrative embodiments, this invention relates to an emulsifier prepared from a plant material selected from a flour, a meal, a fraction, or a whole grain of cereal grains, legumes, a tuber, a root, a stem, a nut, a seed, or other plant material or a combination thereof, comprising one or a plurality of steps of milling and heating said plant material to afford an emulsifier, wherein said emulsifier contains at least a protein component and a carbohydrate component including starch with a starch crystallinity of about 90% or less as compared with the starch crystallinity of the original plant material.

In some illustrative embodiments, this invention relates to an emulsion that contains a flour emulsifier.

In some illustrative embodiments, this invention relates to an emulsion that contains a flour emulsifier. Said emulsion has an oil phase including a lipophilic compound or a combination of lipophilic compounds thereof; wherein said emulsion also contains a water phase which is an aqueous solution, a suspension, or a mixture.

In some illustrative embodiments, this invention relates to an emulsion that contains a flour emulsifier. Wherein said emulsifier contains both protein component and carbohydrate component including starch, wherein the content of protein component is about 1% to about 85% (dry weight base) of the said flour emulsifier composition and the content of carbohydrate component including starch is about 15% to about 99% (dry weight base) of the said flour emulsifier composition, and wherein the crystallinity of the said starch in said flour emulsifier is less than 90% of the crystallinity of the starch in the original plant, and wherein the flour emulsifier composition has emulsification property.

In some illustrative embodiments, this invention relates to an emulsion prepared according to the steps of
a. providing a lipophilic compound or a combination of lipophilic compounds thereof;
b. preparing water or an aqueous solution;
c. preparing an emulsifier or a combination thereof, wherein said emulsifier is prepared from a plant material selected from a flour, a meal, a fraction, or a whole grain of cereal grains, legumes, a tuber, a root, a stem, a nut, a seed, or other plant material or a combination thereof, through one or a plurality of steps of milling and heating of said plant material to afford said emulsifier, wherein said emulsifier contains at least a protein component and a carbohydrate component including starch of reduced crystallinity;
d. mixing components of a., b. and c. to afford a mixture; wherein the preparing and mixing take no particular order;
e. homogenizing said mixture of step d. to afford an emulsion.

In some illustrative embodiments, this invention relates to an encapsulation composition that contains a flour emulsifier component that contains both protein component and carbohydrate component including starch, wherein the content of protein component is about 1% to about 85% (dry weight base) of the said flour emulsifier composition and the content of carbohydrate component including starch is about 15% to about 99% (dry weight base) of the said flour emulsifier composition, and wherein the crystallinity of the said starch in said flour emulsifier is less than 90% of the crystallinity of the starch in the original plant, and wherein the flour emulsifier composition has emulsification property.

In some illustrative embodiments, this invention relates to an encapsulation composition prepared through drying an emulsion that contains an emulsifier prepared from a plant material selected from a flour, a meal, a fraction, or a whole grain of cereal grains, legumes, a tuber, a root, a stem, a nut, a seed, or other plant material or a combination thereof, through one or a plurality of steps of milling and heating of said plant material to afford said emulsifier, wherein said emulsifier contains at least a protein component and a carbohydrate component including starch of reduced crystallinity;

In some illustrative embodiments, this invention relates to using flour emulsifier to make food and beverage, supplement, personal care, cosmetics products, human or animal drug, vaccine, recreational, smoke, inhaled, pharmaceutical, agricultural products, industrial products, or other products. Said products include but not limited to an oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, marijuana or cannabis products allowed by law, animal feed, feed additive, pet food, fish feed, fragrance, marijuana or cannabis containing products allowed by law, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, paint, fabric or leather treatment chemical, plastic, rubber, container, utensil, packaging, tire, construction materials, cloth, fabric, leather and the like, or the ingredient of, or the combination of any of above products.

In some illustrative embodiments, this invention relates to using flour emulsifier in combination with other emulsifiers, including but not limited to small-molecule emulsifiers, modified starch, gum arabic, protein-based emulsifiers, etc.

In some illustrative embodiments, this invention relates to using flour emulsifier in combination with bulking agent, including but not limited to starch-based bulking agent (e.g. maltodextrins, syrups), sugars, sugar alcohols, oligosaccharides, hydrolyzed biopolymers (e.g. polysaccharides hydrolysates, protein hydrolysates), etc.

In some illustrative embodiments, this invention relates to using flour emulsifier in combination with rheological property modifiers, including but not limited to polysaccharide gums, protein-based hydrocolloids, synthetic polymers, etc.

In some illustrative embodiments, this invention relates to using flour emulsifier in combination with various protein resources, including but not limited to dairy proteins, pea proteins, soy proteins, cricket powders, cricket proteins, protein hydrolysates, egg whites, egg powders, egg products, etc.

In some illustrative embodiments, this invention relates to using flour emulsifier in combination with prebiotics and probiotics, including but not limited to probiotics bacteria and their formulations, prebiotics and their formulations, components and formulations related to concepts and applications of microbiome, etc.

In some illustrative embodiments, this invention relates to using flour emulsifier in combination with marijuana or cannabis related material or product.

In one illustrative embodiment, the present invention relates to an emulsifier composition,
(1) Wherein the emulsifier composition is made from a mixture of protein component and carbohydrate component;
(2) wherein the content of protein component is about 1% to about 95% (dry weight base) of the said emulsifier composition and the content of carbohydrate component is about 5% to about 99% (dry weight base) of the said emulsifier composition;
(3) Wherein the emulsifier composition is obtained through subjecting the mixture of protein component and carbohydrate component to milling (2 minutes to 50 hours) and heating (40° C. to 300° C., 2 minutes to 100 hours) in one or a plurality of steps;
(4) Wherein the said milling process provides a power input of not less than 0.05 kilowatts (kw) per kilogram of the said mixture of protein and carbohydrate.
(5) Wherein the said emulsifier composition has an emulsification property.

In this invention, a flour emulsifier refers to a disclosed emulsifier in flour form. A regular flour or raw flour refers to an original plant material in powder form, which is simply a reduction of particle size without any other further treatment on the original plant material disclosed in this invention.

The products of the methods or processes disclosed herein are within the scope of this present invention.

### BRIEF DESCRIPTION OF THE DRAWINGS

The above and other objects, features, and advantages of the present invention will become more apparent when taken in conjunction with the following descriptions and drawings wherein identical reference numerals have been used, where possible, to designate identical features that are common to the figures, and wherein:

FIG. 1. Emulsions prepared with no emulsifier, rice flour emulsifier #1 (RFE-1), untreated rice flour #1 (URF-1), and gum arabic. The images were taken right after shaking (<2 min) and at 30, 60, and 120 minutes after shaking.

FIG. 1 shows the images of homogenized mixtures taken right after shaking (within 2 minutes after shaking) and at 30, 60, and 120 min after the shaking. The image taken at 120 min after the shaking showed that without the use of emulsifier (no emulsifier), the majority of oil droplets moved to the top of aqueous phase. For untreated rice flour #1 (URF-1) stabilized emulsion, both creaming and sedimentation occurred, indicating the low stability of emulsion and thus the lack of emulsification capability of URF-1. For gum arabic stabilized emulsion, a layer of creaming occurred with its thickness much lower than that of no emulsifier and of URF-1 groups. For rice flour emulsifier 1 (RFE-1) stabilized emulsion, the creaming layer was comparable as that of gum arabic, showing a much greater emulsification capability of RFE-1 than URF-1.

FIG. 2. Emulsions prepared with no emulsifier, rice flour emulsifier #2 (RFE-2), untreated rice flour #2 (URF-2), and gum arabic. The images were taken right after shaking (<2 min) and at 30, 60, and 120 minutes after shaking.

FIG. 2 shows the image taken right after shaking (within 2 minutes after shaking) and at 30, 60, and 120 min after tube shaking. For the “120 min” group, it is shown that without the use of emulsifier, the majority of oil droplets moved to the top of aqueous phase. For URF-2 stabilized emulsion, both creaming and sedimentation occurred, indicating the low stability of emulsion and thus the lack of emulsification capability of URF-2. For gum arabic stabilized emulsion, a layer of creaming occurred with its thickness much lower than that of no emulsifier and of URF-2 groups. For RFE-2 stabilized emulsion, the creaming layer was much thinner than that of gum arabic, showing a greater emulsification capability of RFE-2 than URF-2 and gum arabic.

FIG. 3. Emulsions prepared with no emulsifier, rice flour emulsifier #2 (RFE-2-1), untreated rice flour #2 (URF-2), and gum arabic.

FIG. 3 shows the image taken at 120 min after tube shaking. Without the use of emulsifier, the majority of oil droplets moved to the top of aqueous phase. For URF-2 stabilized emulsion, both creaming and sedimentation occurred, indicating the low stability of emulsion and thus the lack of emulsification capability of URF-2. For gum arabic stabilized emulsion, a layer of creaming occurred with its thickness much lower than that of no emulsifier and URF-2 groups. For RFE-2-1 stabilized emulsion, the creaming layer was lighter and thinner than that of gum arabic, showing a greater emulsification capability of RFE-2-1 than URF-2 and gum arabic.

FIG. 4. Emulsions prepared with no emulsifier, rice flour emulsifier #3 (RFE-3), untreated rice flour #3 (URF-3), and gum arabic.

FIG. 4 shows the image taken at 120 min after tube shaking. Without the use of emulsifier, the majority of oil droplets moved to the top of aqueous phase. For URF-3 stabilized emulsion, both creaming and sedimentation occurred, indicating the low stability of emulsion and thus the lack of emulsification capability of URF-3. For gum arabic stabilized emulsion, a layer of creaming occurred with its thickness much lower than that of no emulsifier and URF-3 groups. For RFE-3 stabilized emulsion, the creaming layer was much lighter and thinner than that of URF-3, showing a much enhanced emulsification capability of RFE-3 compared with that of URF-3.

FIG. 5. Emulsions prepared with no emulsifier, barley flour emulsifier (BFE), untreated barley flour (UBF), and gum arabic. The images were taken right after shaking (<2 min) and at 30, 60, and 120 minutes after shaking.

FIG. 5 shows the image taken right after shaking (within 2 min after shaking) and at 30, 60, and 120 min after tube shaking. For the “120 min” group, it is shown that without the use of emulsifier, the majority of oil droplets moved to the top of aqueous phase. For UBF stabilized emulsion, both creaming and sedimentation occurred, indicating the low stability of emulsion and thus the lack of emulsification capability of UBF. For gum arabic stabilized emulsion, a layer of creaming occurred with its thickness much lower than that of no emulsifier and of UBF groups. For BFE stabilized emulsion, the creaming layer was much lighter and thinner than that of UBF and gum arabic, showing a greater emulsification capability of BFE than that of UBF and gum arabic.

FIG. 6. Emulsions prepared with no emulsifier, rice flour emulsifier #4 (RFE-4), untreated rice flour #4 (URF-4), and gum arabic.

FIG. 6 shows the image taken at 120 min after tube shaking. Without the use of emulsifier, the majority of oil droplets moved to the top of aqueous phase. For URF-4 stabilized emulsion, both creaming and sedimentation occurred, indicating the low stability of emulsion and thus the lack of emulsification capability of URF-4. For gum arabic stabilized emulsion, a layer of creaming occurred with its thickness much lower than that of no emulsifier and URF-4 groups. For RFE-4 stabilized emulsion, the creaming layer was much lighter and thinner than that of URF-4 and gum arabic, showing a much greater emulsification capability of RFE-4 than that of gum arabic.

FIG. 7. Emulsions prepared with no emulsifier, whole-wheat flour emulsifier (WFE), untreated whole-wheat flour (UWF), and gum arabic.

FIG. 7 shows the image taken at 120 min after tube shaking. Without the use of emulsifier, the majority of oil droplets moved to the top of aqueous phase. For untreated wheat flour (UWF) stabilized emulsion, both creaming and sedimentation occurred, indicating the low stability of emulsion and thus the lack of emulsification capability of UWF. For gum arabic stabilized emulsion, a layer of creaming occurred with its thickness much lower than that of no emulsifier and UWF groups. For wheat flour emulsifier (WFE) stabilized emulsion, the creaming layer was much lighter and thinner than that of UWF, indicating its higher emulsification capability than that of UWF.

FIG. 8. Emulsions prepared with no emulsifier, degermed corn flour emulsifier #1 (CFE-1), untreated corn flour #1 (UCF-1), and gum arabic.

FIG. 8 shows the image taken at 120 min after tube shaking. Without the use of emulsifier, the majority of oil droplets moved to the top of aqueous phase. For untreated corn flour #1 (UCF-1) stabilized emulsion, both creaming and sedimentation occurred, indicating the low stability of emulsion and thus the lack of emulsification capability of UCF-1. For gum arabic stabilized emulsion, a layer of creaming occurred with its thickness much lower than that of no emulsifier and UCF-1 groups. For corn flour emulsifier 1 (CFE-1) stabilized emulsion, the creaming layer was much lighter and thinner than that of UCF-1 and similar to that of gum arabic, indicating a similar emulsification capability of CFE-1 to that of gum arabic.

FIG. 9. Emulsions prepared with no emulsifier, degermed corn flour emulsifier #2 (CFE-2), untreated corn flour #2 (UCF-2), and gum arabic.

FIG. 9 shows the image taken at 120 min after tube shaking. Without the use of emulsifier, the majority of oil droplets moved to the top of aqueous phase. For UCF-2 stabilized emulsion, substantial creaming occurred, indicating the low stability of emulsion and thus the low emulsification capability of UCF-2. For gum arabic stabilized emulsion, a layer of creaming occurred with its thickness much lower than that of no emulsifier and UCF-2 groups. For CFE-2 stabilized emulsion, the creaming layer was much lighter and thinner than that of UCF-2 and similar to that of gum arabic, indicating a similar emulsification capability of CFE-2 to that of gum arabic.

FIG. 10. Emulsions prepared with no emulsifier, rice flour emulsifier #5 (RFE-5), untreated rice flour #5 (URF-5), and gum arabic.

FIG. 10 shows the image taken at 120 min after tube shaking. Without the use of emulsifier, the majority of oil droplets moved to the top of aqueous phase. For URF-5 stabilized emulsion, substantial creaming occurred, indicating the low stability of emulsion and thus the lack of emulsification capability of URF-5. For gum arabic stabilized emulsion, a layer of creaming occurred with its thickness much lower than that of no emulsifier and URF-5 groups. For RFE-5 stabilized emulsion, the creaming layer was much thinner than that of URF-5 and similar to that of gum arabic, indicating a comparable emulsification capability of RFE-5 with gum arabic.

FIG. 11. Emulsions prepared with no emulsifier, rice flour emulsifier #6 (RFE-6), untreated rice flour #6 (URF-6), and gum arabic.

FIG. 11 shows the image taken at 120 min after tube shaking. Without the use of emulsifier, the majority of oil droplets moved to the top of aqueous phase. For URF-6 stabilized emulsion, substantial creaming and sedimentation occurred, indicating the low stability of emulsion and thus the lack of emulsification capability of URF-6. For gum arabic stabilized emulsion, a layer of creaming occurred with its thickness much lower than that of no emulsifier and URF-6 groups. For RFE-6 stabilized emulsion, the creaming layer was much thinner than that of URF-6 and similar to that of gum arabic, indicating a comparable emulsification capability of RFE-6 with gum arabic.

FIG. 12. Emulsions prepared with no emulsifier, northern bean flour emulsifier (NBFE), untreated northern bean flour (UNBF), and gum arabic.

FIG. 12 shows the image taken at 120 min after tube shaking. Without the use of emulsifier, the majority of oil droplets moved to the top of aqueous phase. For untreated northern bean flour (UNBF) stabilized emulsion, substantial creaming and sedimentation occurred, indicating the low stability of emulsion and thus the lack of emulsification capability of UNBF. For gum arabic stabilized emulsion, a layer of creaming occurred with its thickness much lower than that of no emulsifier and UNBF. For northern bean flour emulsifier (NBFE) stabilized emulsion, the creaming layer was much thinner than that of UNBF, indicating an increased emulsification capability of northern bean flour due to the combined heating and milling treatment.

FIG. 13. Emulsions prepared with no emulsifier, kidney bean flour emulsifier (KBFE), untreated kidney bean flour (UKBF), and gum arabic.

FIG. 13 shows the image taken at 120 min after tube shaking. Without the use of emulsifier, the majority of oil droplets moved to the top of aqueous phase. For untreated kidney bean flour (UKBF) stabilized emulsion, substantial creaming and sedimentation occurred, indicating the low stability of emulsion and thus the lack of emulsification capability of UKBF. For gum arabic stabilized emulsion, a layer of creaming occurred with its thickness much lower than that of no emulsifier and UKBF. For kidney bean flour (KBFE) stabilized emulsion, the creaming layer was lighter than that of UKBF, indicating an increased emulsification capability of kidney bean flour due to the combined heating and milling treatment.

FIG. 14. Emulsions prepared with no emulsifier, rice flour emulsifier #2-2 (RFE-2-2), untreated rice flour #2 (URF-2), and gum arabic.

FIG. 14 shows the image taken at 120 min after tube shaking. Without the use of emulsifier, the majority of oil droplets moved to the top of aqueous phase. For URF-2 stabilized emulsion, both creaming and sedimentation occurred, indicating the low stability of emulsion and thus the lack of emulsification capability of URF-2. For gum arabic stabilized emulsion, a layer of creaming occurred with its thickness much lower than that of no emulsifier and URF-2 groups. For RFE-2-2 stabilized emulsion, the creaming layer was lighter and thinner than that of gum arabic, showing a greater emulsification capability of RFE-2-2 than URF-2 and gum arabic.

FIG. 15. Emulsions of retinol-soybean oil mixture prepared with no emulsifier, rice flour emulsifier #2-2 (RFE-2-2), and gum arabic.

FIG. 15 shows the image taken at 120 min after tube shaking. Without the use of emulsifier, the majority of oil droplets moved to the top of aqueous phase. For gum arabic stabilized emulsion, a light layer of creaming occurred with its thickness much lower than that of no emulsifier. For RFE-2-2 stabilized emulsion, the creaming layer was nearly invisible, showing a greater emulsification capability of RFE-2-2 than gum arabic for retinol-soybean oil mixture.

FIG. 16. Emulsions of tocopherol prepared with no emulsifier, rice flour emulsifier #2-2 (RFE-2-2), and gum arabic.

FIG. 16 shows the image taken at 120 min after tube shaking. Without the use of emulsifier, the majority of oil droplets separated from the water phase. For gum arabic stabilized emulsion, a layer of creaming occurred. For RFE-2-2 stabilized emulsion, the creaming layer also formed, however, with lower density, showing the emulsification capability of RFE-2-2 comparable with or superior to that of gum arabic for tocopherol.

FIG. 17. Emulsions of astaxanthin oleoresin prepared with no emulsifier, rice flour emulsifier #2-2 (RFE-2-2), and gum arabic.

FIG. 17 shows the image taken at 120 min after tube shaking. Without the use of emulsifier, the astaxanthin oleoresin cannot be well dispersed and thus form oily paste dots along the wall of tube. For gum arabic, the oleoresin was better dispersed but a substantial amount of oily paste still attached at the wall surface. For RFE-2-2, the oily paste was nearly negligible due to a much more even dispersion formed. Apparently, RFE-2-2 has a much greater capability to disperse and emulsify astaxanthin oleoresin than gum arabic.

FIG. 18. Color of emulsions of paprika oleoresin stabilized using rice flour emulsifier #2-2 (RFE-2-2) and gum arabic.

FIG. 18 shows that both REF-2-2 and gum arabic were able to form emulsions of paprika oleoresin. After 48 h exposure to light, the color strength of gum arabic stabilized emulsion was substantially reduced, whereas the color of RFE-2-2 stabilized emulsion nearly retained its original strength. Therefore, the used of REF-2-2 as an emulsifier was able to protect the coloring component in paprika oleoresin from being degraded by the light exposure.

FIG. 19. Emulsions prepared with rice flour emulsifier #2 (RFE-2) and rice flour treated with ball milling only (URF-2B) right after shaking and at 30, 60, and 120 min after shaking.

FIG. 19 shows the photographs taken right after shaking (within 2 minutes after shaking) and at 30, 60, and 120 min after shaking. It is shown that at any point, the emulsion formed with RFE-2 was much more stable than the emulsion formed with URF-2B, demonstrating the role of combined heating and milling on producing flour emulsifier with superior emulsification properties, compared with milling-only treatment.

FIG. 20. Emulsions prepared with rice flour emulsifier #2 (RFE-2) and treated rice starch (Starch-HB) right after shaking and at 30, 60, and 120 min after shaking.

FIG. 20 shows the images of homogenized mixtures taken right after shaking (within 2 min after shaking) and at 30, 60, and 120 min after shaking. It is shown that at any point after shaking, Starch-HB was not able to form emulsion. Even right after the shaking, the oil-water separation occurred immediately. This shows that the emulsification capability of Starch-HB was negligible. In contrast, RFE-2 stabilized the emulsion all through 120 min after shaking, highlighting the importance of protein components in the flour for providing acceptable emulsification properties.

FIG. 21. Photographs of emulsions subjected to freeze-thaw treatment. The type of emulsifiers used included rice flour not subjected to heating and milling (raw flour), rice flour subjected to heating and milling (flour emulsifier, FE1), and OSA-starch (OSA-starch).

As shown in FIG. 21, both FE1 and OSA-starch were able to form emulsions. In contrast, rice flour not subjected to heating and milling could not form emulsion effectively. For emulsions subjected to freeze-thaw treatment, emulsion formed with OSA-starch showed a thick oil layer, whereas no noticeable oil layer was found with emulsion formed with FE1. The result indicated that the freeze-thaw stability of emulsion formed with FE1 was much higher than that of the emulsion formed with regular rice flour and the emulsion formed with OSA-starch.

FIG. 22. Photograph showing the appearance of coffee drink added with FE1 creamer or a commercial creamer, with black coffee as comparison.

As shown in FIG. 22, the coffee creamer prepared using flour emulsifier FE1 showed essentially the same whitening capability as that shown by the commercial creamer. Based on the sensory tests, both FE1-containing creamer and commercial creamer performed similarly in providing creamy mouthfeel and masking the bitterness and acidity originally in black coffee.

FIG. 23. Microscopic images of oil droplets in almond milk. Left: almond milk without added emulsifier. Right: almond milk with FE1. Magnification: 400×. Length of black needle: 220 μm.

As shown in FIG. 23, the almond milk with FE1 showed much smaller oil droplets than those in the almond milk without added emulsifier, suggesting the capability of FE1 to stabilize almond milk as an emulsion.

FIG. 24. Photograph of emulsion of black pepper oleoresin formed without emulsifier (left) or with FE1 as emulsifier (right).

As shown in FIG. 24, a stable emulsion of black pepper oleoresin was formed using FE1 as the emulsifier.

FIG. 25. Photographs of French dressings (top) and microscopic images of oil droplets in diluted dressings (bottom). For microscopic images, magnification was 400× and the length of black needle was 220 μm. Raw flour: rice flour not subjected to heating and milling. FE1: flour emulsifier (rice flour subjected to heating and milling).

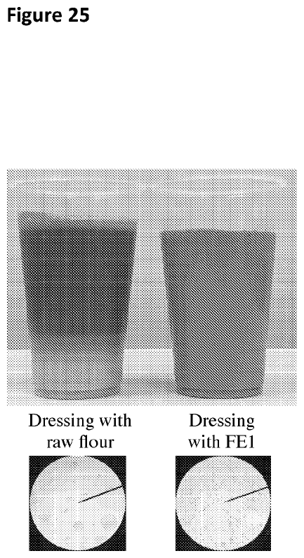

As shown in FIG. 25, the dressing made with raw flour showed layer separation, whereas the dressing made with FE1 was uniform and stable. In addition, the oil droplets of dressing formed with FE1 were much smaller than those of dressing formed with raw flour. Such a result indicates that FE1 is an effective emulsifier for preparing French dressing.

### DETAILED DESCRIPTION

For the purposes of promoting an understanding of the principles of the present disclosure, reference will now be made to the embodiments illustrated in the drawings and examples, and specific language will be used to describe the same. It will nevertheless be understood that no limitation of the scope of this disclosure is thereby intended.

In the present disclosure, the term “about” can allow for a degree of variability in a value or range, for example, within 20%, within 10%, within 5%, or within 1% of a stated value or of a stated limit of a range.

In the present disclosure, the term “substantially” can allow for a degree of variability in a value or range, for example, within 80%, within 90%, within 95%, or within 99% of a stated value or of a stated limit of a range.

The present application relates generally to flour emulsifiers, methods of production thereof, their use in emulsions, their use in food and non-food products, formulations or products containing said emulsifiers, and specifically to emulsifiers prepared using renewable products of agriculture through a physical process. The flour emulsifiers are made from plant sources without chemical treatment. The flour emulsifiers can be used in both food and non-food applications. While the flour emulsifiers have a basic form of flour, they can be produced, processed, or formulated in other forms such as particulates, grits, granules, powders, solutions, dispersions, suspensions, or mixture with other ingredients that are suitable for specific applications. The flour emulsifiers primarily provide emulsification properties, and in certain circumstances and applications, they may also provide other functionalities including but not limited to bulking, stabilizing, texturizing, formulating, and or protecting in food and non-food systems.

In some other embodiments, this invention relates to an emulsifier, wherein said emulsifier is made from a flour, a meal, a fraction, or a whole grain of a cereal grain, a legume, a tuber, a root, a stem, a seed, a nut, or other plant material, or a combination thereof.

In this invention, an emulsifier is described, wherein said emulsifier is made from a flour, a meal, a fraction, or a whole grain of a cereal grain, a legume, a tuber, a root, a stem, a nut, a seed, or other plant material, or a combination thereof. Said flour emulsifier has emulsification properties.

In some other embodiments, this invention relates to an emulsifier, wherein said emulsifier has protein component from about 1% to about 85% of said emulsifier.

In some other embodiments, this invention relates to an emulsifier, wherein said emulsifier has carbohydrate component from about 15% to about 99% of said emulsifier.

In some other embodiments, this invention relates to an emulsifier, wherein said emulsifier is a flour originated from one or a mixture of plant materials.

In some other embodiments, this invention relates to a flour emulsifier, wherein the crystallinity of the starch component of said flour emulsifier ranges from about 0% to about 70%.

In this invention, an emulsifier is described. The emulsifier is made from a flour, a meal, a fraction, or a whole grain of a cereal grain, a legume, a tuber, a root, a stem, a seed, a nut, or other plant material, or a combination thereof, wherein said emulsifier comprises at least a protein component and a carbohydrate component including starch, and wherein the crystallinity of the said starch is less than 90% of the crystallinity of the starch in the original plant material.

The major cereals are wheat, corn, rice, barley, sorghum, millet, oats, and rye. Botanically, cereals are grasses and belong to the monocot family Poaceae. Wheat, rye, and barley are closely related as members of the subfamily Pooideae and the tribus Triticeae. Oats are a distant relative of the Triticeae within the subfamily Pooideae. In general, cereals generate dry, one-seeded fruits called kernel or grain in the form of a caryopsis. The fruit coat (pericarp) is bound to the seed coat (testa). The size and weight of a grain vary from large corn grains (˜350 mg) to small millet grains (˜9 mg) (Koehler & Wieser. Chapter 2: Chemistry of Cereal Grains, from “Handbook on Sourdough Biotechnology” by Gobbetti & Ganzle, 2013). The anatomy of cereal grains is rather uniform, in which the germ and the endosperm are enclosed within the fruit and seed coats (bran) and consist of the starchy endosperm and the aleurone layer (Koehler & Wieser. Chapter 2: Chemistry of Cereal Grains, from “Handbook on Sourdough Biotechnology” by Gobbetti & Ganzle, 2013).

Cereal grains contain high amount of carbohydrates with starch mainly deposited in the endosperm and fiber mainly located in the bran. In the grains, the average content of protein ranges about 8-13%. The content of lipids in cereal grains is usually about (2-4%) and the content of minerals is about 1-3%. In addition, cereal grains contain various vitamins, in particular B-vitamins (Koehler & Wieser. Chapter 2: Chemistry of Cereal Grains, from “Handbook on Sourdough Biotechnology” by Gobbetti & Ganzle, 2013).

Table 1 lists the composition of several cereal grains, while it is understood that the actual amounts of individual components may vary depending on factors such as their genotypes, varieties, growth conditions, and growth locations, etc.

TABLE 1
Average values of cereal compositions (Belitz, Grosch, and Schieberle. Chapter
15: Cereal and Cereal Products, from “Food Chemistry”, Springer 2009)

| | Wheat | Rye | Corn | Barley | Oats | Rice | Millet |
|---|---|---|---|---|---|---|---|
| | weight % | | | | | | |
| Moisture | 13.2 | 13.7 | 12.5 | 11.7 | 13.0 | 13.1 | 12.1 |
| Protein (N × 6.25) | 11.7 | 9.5 | 9.2 | 10.6 | 12.6 | 7.4 | 10.6 |
| Lipids | 2.2 | 1.7 | 3.8 | 2.1 | 7.1 | 2.4^{a} | 4.05 |
| Available carbohydrates | 59.6 | 60.7 | 64.2 | 63.3 | 55.7 | 74.1 | 68.8 |
| Fiber | 13.3 | 13.2 | 9.7 | 9.8 | 9.7 | 2.2 | 3.8 |
| Minerals | 1.5 | 1.9 | 1.30 | 2.25 | 2.85 | 1.2 | 1.6 |
| | mg/kg | | | | | | |
| Thiamine | 5.5 | 4.4 | 4.6 | 5.7 | 7.0 | 3.4 | 4.6 |
| Niacin | 63.6 | 15.0 | 26.6 | 64.5 | 17.8 | 54.1 | 48.4 |
| Riboflavin | 1.3 | 1.8 | 1.3 | 2.2 | 1.8 | 0.55 | 1.5 |
| Pantothenic acid | 13.6 | 7.7 | 5.9 | 7.3 | 14.5 | 7.0 | 12.5 |

^{a}Polished rice: 0.8%.

Buckwheat (Fagopyrum esculentum) is a plant cultivated for its grain-like seeds and is a domesticated food plant common in Asia and Central and Eastern Europe. Buckwheat is not related to wheat, as it is not a grass. Buckwheat, with seeds rich in carbohydrates and protein, is a plant material related to this invention.

In general, the term “cereal” in this invention covers: (1) cereals which include but not limited to barley, fonio, Job's tears, maize (corn), millets, oats, rice, rye, sorghum, teff, triticale, wild rice, and wheat and (2) pseudocereals which include but not limited to buckwheat, tartary buckwheat, amaranth and its family, quinoa, pitseed goosefoot, canihua, and chia.

“Legumes” or “pulses” are ripe seeds of the plant family Fabaceae and are an important protein source for human food. The primary components of legumes include protein, carbohydrates, dietary fibers, lipids, vitamins, and minerals, as well as phytoestrogens, and saponins. Well-known legumes (or pulses) include alfalfa, clover, peas, beans, chickpeas, lentils, lupin bean, mesquite, carob, soybeans, peanuts, and tamarind. A partial list of legumes or pulses also includes: kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, lentil, Bambara groundnut, earth pea, vetch, common vetch, lupins, lablab, hyacinth bean, jack bean, sword bean, winged bean, vevet bean, cowitch, and yam bean.

Compared with cereals, legumes contain relatively high amount of protein (Table 2). In contrast, the starch content in legumes are relatively low compared with cereals (Table 3),

TABLE 2
Average values of legume compositions (Belitz, Grosch, and Schieberle. Chapter
16: Legumes, from “Food Chemistry”, Springer 2009)

| | | Crude | | Available | Dietary | |
|---|---|---|---|---|---|---|
| | | protein^{b} | Lipid | carbohydrates | fiber | Minerals |
| Name | Systematic name | (%) | (%) | (%) | ( %) | ( %) |
| Soybeans | Glycine hyspida max | 41.0 | 19.6 | 7.6 | 24.0 | 5.5 |
| Peanuts | Arachis hypogaea | 31.4 | 50.7 | 7.9 | 12.3 | 2.7 |
| Peas | Pisum sativum | 25.7 | 1.4 | 53.7 | 18.7 | 3.0 |
| Garden beans | Phaseolus vulgaris | 24.1 | 1.8 | 54.1 | 19.2 | 4.4 |
| Runner beans | Phaseolus coccineus | 23.1 | 2.1 | n.a. | n.a. | 3.9 |
| Black gram | Phaseolus mungo | 26.9 | 1.6 | 46.3 | n.a. | 3.6 |
| Green gram | Phaseolus aureus | 26.7 | 1.3 | 51.7 | 21.7 | 3.8 |
| (mungo beans) | | | | | | |
| Lima beans | Phaseolus lunatus | 25.0 | 1.6 | n.a. | n.a. | 3.9 |
| Chick peas | Cicer arietinum | 22.7 | 5.0 | 54.6 | 10.7 | 3.0 |
| Broad beans | Vicia faba | 26.7 | 2.3 | n.a. | n.a. | 3.6 |
| Lentils | Lens culinaris | 28.6 | 1.6 | 57.6 | 11.9 | 3.6 |

^{a}The result are average values given as weight-%/dry matter.
^{b}N × 6.25.
n.a.: not analyzed.

TABLE 3
Carbohydrates in legume flours (Belitz, Grosch, and Schieberle.
Chapter 16: Legumes, from “Food Chemistry”, Springer 2009)

| | Glu- | Saccha- | Raffi- | Stach- | Verbas- | |
|---|---|---|---|---|---|---|
| Flour | cose | rose | nose | yose | cose | Starch |
| Garden beans | 0.04 | 2.23 | 0.41 | 2.59 | 0.13 | 51.6 |
| Broad beans | 0.34 | 1.55 | 0.24 | 0.80 | 1.94 | 52.7 |
| Lentils | 0.07 | 1.81 | 0.39 | 1.85 | 1.20 | 52.3 |
| Green gram | 0.05 | 1.28 | 0.32 | 1.65 | 2.77 | 52.0 |
| (mungo beans) | | | | | | |
| Soybean^{b} | 0.01 | 4.5 | 1.1 | 3.7 | | 0.62 |

^{a}Weight—% of the dry matter.
^{b}Defatted flour.

Other plant materials that contain protein and starch include but are not limited to seeds and nuts. Nuts herein include but are not limited to botanical nuts and culinary nuts. Nuts include but are not limited to almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, etc. Table 4 shows the contents of some primary components among several nuts.

TABLE 4
contents of some primary components of several nuts

| | | Total | | Poly- | Mono- | Carbo- |
|---|---|---|---|---|---|---|
| | Protein, | fat, | Saturated | unsaturated | unsaturated | hydrate, |
| Name | % | % | fat, % | fat, % | fat, % | % |
| Almonds | 21.26 | 50.64 | 3.881 | 12.214 | 32.155 | 28.1 |
| Peanuts | 23.68 | 49.66 | 6.893 | 15.694 | 24.64 | 26.66 |
| Pistachio | 20.61 | 44.44 | 5.44 | 13.455 | 23.319 | 34.95 |
| Walnuts | 15.23 | 65.21 | 6.126 | 47.174 | 8.933 | 19.56 |

Other plant materials that contain protein and starch include but are not limited to chestnut, water chestnut, potato, cassava or tapioca, taro, arrowroot, sweet potato, yam, Chinese yam, lotus root, and other plant species or agricultural crops.

Starch, a group of alpha-D-glucans, is a major component among the most abundant plant products. Starch is a major food component providing a bulk nutrient and energy source, and also has been broadly used in the industry to bring various functionalities. In general, native starch exists in the granular form. There are two types of alpha-D-glucans in starch, amylose and amylopectin. Amylose is essentially a linear biopolymer of glucosyl units connected through 1,4-alpha-D glucosidic linkages. In amylose molecules, there can be a small number of branches attaching to the main linear chains through 1,6-alpha-D glucosidic linkages. In contrast, amylopectin is a highly-branched glucan molecule that contains both 1,4 and 1,6-alpha-D glucosidic linkages. It is considered that the branches of amylopectin are arranged in a cluster pattern to form the crystalline structure found in starch granules. The ratio between amylose and amylopectin, as well as the specific structure of amylose and amylopectin are affected by the species, variety, genetic background, and growth conditions of the starch-generating plant.

It is known that starch granules contain crystalline structure. The abundance of such crystalline structure is in general quantified using the degree of crystallinity or in short, crystallinity. Conceptually, starch granules contain both crystalline regions and amorphous regions, and the crystallinity can be defined as the following:

Crystallinity (%)=(amount of crystalline region)/(amount of crystalline region+amount of amorphous region)

Various techniques can be used to determine the crystallinity of starch, and the mostly used method is the X-ray powder diffraction. Three different types of crystal structures have been identified, classified as A-type characteristic of cereal starches, B-type of tuber starches, and C-type with legumes. In addition, it is considered that C-type is a mixture of A- and B-type. Usually, an X-ray powder diffraction crystallogram of a native starch depicts broadened diffraction lines and an underlying band of amorphous (noncrystalline) scattering. These features reflect both the partial crystallinity of granules and diffraction from small or imperfectly organized crystallites (Zobel & Stephen. Chapter 2: Starch: Structure, Analysis, and Application, from “Food Polysaccharides and Their Applications”, Second Edition, by Stephen, Phillips, and Williams, 2006).

In some other embodiments, this invention relates to an emulsifier composition made from one or a mixture of cereal grains, legumes, tubers, roots, stems, seeds, nuts, or other plant materials, wherein said emulsifier composition is able to form an emulsion.

Emulsions normally contain a water phase, an oil phase, and one or more emulsifiers.

In some illustrative embodiments, this invention relates to an emulsifier made from a flour, a meal, a fraction, or a whole grain of a cereal grain, a legume, tubers, roots, stems, a nut, a seed, or other plant material, or a combination thereof, wherein said emulsifier comprises at least a protein component and a carbohydrate component.

In some other embodiments, this invention relates to an emulsifier made from plant materials, wherein said plant materials comprise one or more from wheat, corn, rice, barley, sorghum, millet, oats, rye, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taco, tuber, tapioca, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, or the combination thereof.

In some other embodiments, this invention relates to an emulsifier, which is a flour of one or a mixture of cereal grains, legumes, seeds, nuts, or other plant materials, wherein said emulsifier contains about 1% to 85% protein, about 15% to about 99% carbohydrate, wherein said emulsifier composition is able to form an emulsion.

In some other embodiments, this invention relates to an emulsifier in the form of a flour made from a flour, a meal, a fraction, or a whole grain of a cereal grain, a legume, a tuber, a root, a stem, a seed, a nut, or other plant material, or a combination thereof, wherein said emulsifier contains about 1% to about 85% protein, about 15% to about 99% carbohydrate including starch, and wherein the crystallinity of starch is less than 90% of the crystallinity of starch in the original plant material.

In general, the emulsification capability of an emulsifier is evaluated using the stability of the emulsion formed by said emulsifier. Under specific conditions, the greater emulsion stability an emulsifier can generate, the greater emulsification capability this emulsifier shows. Method for evaluating the stability of emulsion is selected from one or more of the following:
a. The average size (defined by the average diameter) of the oil droplets in the said emulsion is not greater than 1000 μm, 500 μm, 100 μm, 50 μm, 30 μm, 10 μm, 5 μm, 1 μm, 0.5 μm, 0.2 μm, 0.1 μm, or 0.05 μm. The lower value of the average size of the oil droplets indicates greater emulsion stability;
b. The portion (by volume, weight, or number) of the oil droplets with size (diameter) greater than 500 μm is not greater than 95%, 90%, 80%, 70%, 60%, 50%, 40%, 30%, 20%, 10%, 5%, 2%, or 1%. The lower value of the portion of the oil droplets with size greater than 500 μm indicates greater emulsion stability;
c. The portion (by volume, weight, or number) of the oil droplets with size (diameter) greater than 50 μm is not greater than 95%, 90%, 80%, 70%, 60%, 50%, 40%, 30%, 20%, 10%, 5%, 2%, or 1%. The lower value of the portion of the oil droplets with size greater than 50 μm indicates greater emulsion stability;
d. The portion (by volume, weight, or number) of the oil droplets with size (diameter) greater than 10 μm is not greater than 95%, 90%, 80%, 70%, 60%, 50%, 40%, 30%, 20%, 10%, 5%, 2%, or 1%. The lower value of the portion of the oil droplets with size greater than 50 μm indicates greater emulsion stability;
e. The portion (by volume, weight, or number) of the oil droplets with size (diameter) greater than 5 μm is not greater than 95%, 90%, 80%, 70%, 60%, 50%, 40%, 30%, 20%, 10%, 5%, 2%, or 1%. The lower value of the portion of the oil droplets with size greater than Sum indicates greater emulsion stability;
f. The portion (by volume, weight, or number) of the oil droplets with size (diameter) greater than 1 μm is not greater than 95%, 90%, 80%, 70%, 60%, 50%, 40%, 30%, 20%, 10%, 5%, 2%, or 1%. The lower value of the portion of the oil droplets with size greater than 1 μm indicates greater emulsion stability;
g. Under about one g-force (e.g. regular gravity. One g or one g-force is the acceleration due to gravity at the Earth's surface and is the standard gravity, defined as 9.80665 metres per second squared, or equivalently 9.80665 newtons of force per kilogram of mass), the visible separation in the emulsion, including but not limited to creaming, flocculation, aggregation, sedimentation, and precipitation, occurs at least after 0.1 min, 0.5 min, 1 min, 5 min, 10 min, 20 min, 50 min, 100 min, 200 min, 500 min, 1000 min, 2000 min, 5000 min, 10,000 min, 20,000 min, 50,000 min, or 100,000 min. The greater time needed for visible separation to occur indicates greater emulsion stability;
h. After subjecting the emulsion to centrifugation for 1 minute, the visible separation in the emulsion, including but not limited to creaming, flocculation, aggregation, sedimentation, and precipitation, occurs at a centrifugal force of at least 1×g, 2×g, 5×g, 10×g, 20×g, 50×g, 100×g, 200×g, 500×g, 1000×g, 2000×g, 5000×g, 10,000×g, 20,000×g, and 50,000×g (relative centrifugal force (RCF) is the term used to describe the amount of accelerative force applied to a sample in a centrifuge. RCF is measured in multiples of the standard acceleration due to gravity at the Earth's surface (×g). This is why RCF and “×g” are used interchangeably in centrifugation protocols). The greater centrifugal force needed for visible separation to occur indicates greater emulsion stability;
i. After subjecting the emulsion to centrifugation for 10 minutes, the visible separation in the emulsion, including but not limited to creaming, flocculation, aggregation, sedimentation, and precipitation, occurs at a centrifugal force of at least 1×g, 2×g, 5×g, 10×g, 20×g, 50×g, 100×g, 200×g, 500×g, 1000×g, 2000×g, 5000×g, 10,000×g, 20,000×g, and 50,000×g. The greater centrifugal force needed for visible separation to occur indicates greater emulsion stability;
j. After subjecting the emulsion to centrifugation for a specific period of time, the visible separation in the emulsion, including but not limited to creaming, flocculation, aggregation, sedimentation, and precipitation, occurs at a centrifugal force of at least 1×g, 2×g, 5×g, 10×g, 20×g, 50×g, 100×g, 200×g, 500×g, 1000×g, 2000×g, 5000×g, 10,000×g, 20,000×g, and 50,000×g. The greater centrifugal force needed for visible separation to occur indicates greater emulsion stability.

In some other embodiments, this invention relates to an emulsifier composition made from one or a mixture of cereal grains, legumes, tubers, roots, stems, seeds, nuts, or other plant materials, or a combination thereof, wherein said emulsifier contains about 1% to 85% protein, about 15% to 99% carbohydrate, wherein said emulsifier composition is able to form an emulsion at the emulsifier composition-to-oil ratio of 1/100 to 100/1, preferentially of 1/10 to 10/1, wherein the emulsion is characterized by at least one of the following methods:
a. The average size (defined by the average diameter) of the oil droplets in the said emulsion is not greater than 1000 μm, 500 μm, 100 μm, 50 μm, 30 μm, 10 μm, 5 μm, 1 μm, 0.5 μm, 0.2 μm, 0.1 μm, or 0.05 μm;
b. The portion (by volume, weight, or number) of the oil droplets with size (diameter) greater than 500 μm is not greater than 95%, 90%, 80%, 70%, 60%, 50%, 40%, 30%, 20%, 10%, 5%, 2%, or 1%;
c. The portion (by volume, weight, or number) of the oil droplets with size (diameter) greater than 50 μm is not greater than 95%, 90%, 80%, 70%, 60%, 50%, 40%, 30%, 20%, 10%, 5%, 2%, or 1%;

d. The portion (by volume, weight, or number) of the oil droplets with size (diameter) greater than 10 μm is not greater than 95%, 90%, 80%, 70%, 60%, 50%, 40%, 30%, 20%, 10%, 5%, 2%, or 1%;
e. The portion (by volume, weight, or number) of the oil droplets with size (diameter) greater than Sum is not greater than 95%, 90%, 80%, 70%, 60%, 50%, 40%, 30%, 20%, 10%, 5%, 2%, or 1%;
f. The portion (by volume, weight, or number) of the oil droplets with size (diameter) greater than 1 μm is not greater than 95%, 90%, 80%, 70%, 60%, 50%, 40%, 30%, 20%, 10%, 5%, 2%, or 1%;
g. Under about one g-force (e.g. regular gravity. One g or one g-force is the acceleration due to gravity at the Earth's surface and is the standard gravity, defined as 9.80665 metres per second squared, or equivalently 9.80665 newtons of force per kilogram of mass), the visible separation in the emulsion, including but not limited to creaming, flocculation, aggregation, sedimentation, and precipitation, occurs at least after 0.1 min, 0.5 min, 1 min, 5 min, 10 min, 20 min, 50 min, 100 min, 200 min, 500 min, 1000 min, 2000 min, 5000 min, 10,000 min, 20,000 min, 50,000 min, or 100,000 min;
h. After subjecting the emulsion to centrifugation for 1 minute, the visible separation in the emulsion, including but not limited to creaming, flocculation, aggregation, sedimentation, and precipitation, occurs at a centrifugal force of at least 1×g, 2×g, 5×g, 10×g, 20×g, 50×g, 100×g, 200×g, 500×g, 1000×g, 2000×g, 5000×g, 10,000×g, 20,000×g, and 50,000×g (relative centrifugal force (RCF) is the term used to describe the amount of accelerative force applied to a sample in a centrifuge. RCF is measured in multiples of the standard acceleration due to gravity at the Earth's surface (×g). This is why RCF and “×g” are used interchangeably in centrifugation protocols);
i. After subjecting the emulsion to centrifugation for 10 minutes, the visible separation in the emulsion, including but not limited to creaming, flocculation, aggregation, sedimentation, and precipitation, occurs at a centrifugal force of at least 1×g, 2×g, 5×g, 10×g, 20×g, 50×g, 100×g, 200×g, 500×g, 1000×g, 2000×g, 5000×g, 10,000×g, 20,000×g, and 50,000×g;
j. After subjecting the emulsion to centrifugation for a specific period of time, the visible separation in the emulsion, including but not limited to creaming, flocculation, aggregation, sedimentation, and precipitation, occurs at a centrifugal force of at least 1×g, 2×g, 5×g, 10×g, 20×g, 50×g, 100×g, 200×g, 500×g, 1000×g, 2000×g, 5000×g, 10,000×g, 20,000×g, and 50,000×g.

One emulsion can also be compared with another emulsion to determine their relative stabilities. The comparison needs to be conducted at the same environmental conditions including but not limited to the same temperatures, gravity or centrifugation forces, containers, and light exposures. The parameters that can be used for comparison include but are limited to:
a. The average particle size of oil droplets in the emulsion;
b. The portion of oil droplets with size greater than a specific level;
c. The time needed for emulsion to show visible separation under regular gravity force or a specific centrifugation force;
d. The centrifugation force needed for emulsion to show visible separation over a specific centrifugation time;
e. A combination of parameters described in (a), (b), (c), and (d) above.

In some other embodiments, a comparison on emulsion stability is needed between different emulsions, thus to describe the emulsification property of different emulsifiers contained in those emulsions. For example, a comparison of emulsion stability is needed between emulsion A and emulsion B. For such a comparison, both emulsion A and emulsion B need to be subjected to defined environmental conditions including but not limited to temperatures, gravity or centrifugation forces, containers, and light exposures. Thereafter, the properties of emulsion A and emulsion B are compared. If one or more than one outcomes listed below occur, then emulsion A is considered more stable than emulsion B:
a. After emulsion preparation, both emulsion A and emulsion B are subjected to the same environmental conditions for the same period of time. Thereafter, the particle sizes of oil droplets are measured for emulsions. The results show that the average particle size of emulsion A is lower than that of emulsion B;
b. After emulsion preparation, both emulsion A and emulsion B are subjected to the same environmental conditions for the same period of time. Thereafter, the particle size distributions of oil droplets are measured for emulsions, and the portions of oil droplets with sizes greater than a specific value are determined as portion A and portion B for emulsion A and emulsion B, respectively. The results show that portion A is lower than portion B;
c. After emulsion preparation, both emulsion A and emulsion B are subjected to the same environmental conditions. It takes emulsion A longer time than emulsion B to reach the same or essentially the same separation level.
d. After emulsion preparation, emulsion A and emulsion B are subjected to different centrifugation forces for the same period of time to reach the same or essentially the same separation levels. The centrifugation force needed for emulsion A is greater than that needed for emulsion B.

In some other embodiments, the relative stability between different emulsions can be quantified. In this invention, the time needed for an emulsion to reach a specific level of separation is used to quantitatively characterize the stability of the emulsion. After the preparations of emulsion A and emulsion B, both are subjected to the same or essentially the same environmental conditions to allow for separation to occur. If one or more than one outcomes listed below occur, then the stability of emulsion A is considered to be at least N times that of the stability of emulsion B:
(1) The time duration needed for emulsion A to reach a specific separation (t_{A}) is N times the time duration needed for emulsion B to reach the same specific separation (t_{B}), that is, t_{A}=N t_{B};
(2) The time duration needed for emulsion A to reach a specific separation (t_{A}) is more than N times the time duration needed for emulsion B to reach the same specific separation (t_{B}), that is, t_{A}>N t_{B};
(3) The time duration needed for emulsion A to reach a specific separation (t_{A}) is more than N times the time duration needed for emulsion B to reach the same specific separation (t_{B}). However, to value is too large to be determined practically. In this scenario, t_{A}>>N t_{B};

For an emulsion, the time duration after the emulsion preparation can be defined by one of the following:
(1) The time passed after the emulsion is freshly prepared through agitation, shaking, sonication (i.e. treated with ultrasound), or homogenization of a mixture of oil and aqueous solvent, or through rehydration of a dried emulsion solid such as an encapsulation solid;
(2) The time passed after re-agitating, re-shaking, re-sonicating, or re-homogenizing a prepared emulsion to re-disperse the oil droplets;

With above comparison for different emulsions, the properties of emulsifiers forming said emulsions are described and compared. Under specific conditions, the emulsifier forming emulsion A described above has greater emulsification capability than the emulsifier forming emulsion B.

In some other embodiments, this invention relates to a flour emulsifier made from one or a mixture of cereal grains, legumes, seeds, nuts, or other plant materials, wherein said flour emulsifier has emulsification capability at least 2 times that of the regular flour from the same plant material.

In some other embodiments, this invention relates to a flour emulsifier made from one or a mixture of cereal grains, legumes, seeds, nuts, or other plant materials, wherein said flour emulsifier has emulsification capability at least 3 times that of the regular flour from the same plant material.

In some other embodiments, this invention relates to a flour emulsifier made from one or a mixture of cereal grains, legumes, seeds, nuts, or other plant materials, wherein said flour emulsifier has emulsification capability at least 5 times that of the regular flour from the same plant material.

In some other embodiments, this invention relates to a flour emulsifier made from one or a mixture of cereal grains, legumes, seeds, nuts, or other plant materials, wherein said flour emulsifier has emulsification capability at least 10 times that of the regular flour from the same plant material.

In some other embodiments, this invention relates to an emulsifier described herein, said emulsifier is used in a product. Said product is a food, food ingredient, beverage, personal care product, cosmetics, medical product, drug, industrial product, agricultural product, or other product alike.

In some other embodiments, this invention relates to an emulsifier described herein, said emulsifier is used in a product. Said product is a oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, animal feed, feed additive, pet food, fish feed, fragrance, marijuana or cannabis containing products allowed by law, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, paint, fabric or leather treatment chemical, plastic, rubber, container, utensil, packaging, tire, cloth, fabric, leather, or the ingredients of, or combinations of any of above products and the like.

In some other embodiments, this invention relates to an emulsifier described herein, said emulsifier also provide thickening, texture improving, creaminess, improved mouthfeel, improved freeze-thaw stability, improved physical stability, improved chemical stability, and/or other properties to an emulsion. For example, the use of said emulsifier may lead to higher viscosity or gel strength of an emulsion-related system or product.

In some other embodiments, this invention relates to an emulsifier described herein, said emulsifier also provide thickening, texture improving, creaminess, improved mouthfeel, improved freeze-thaw stability, improved physical stability, improved chemical stability, and/or other properties to a product. Said product is a food, food ingredient, beverage, personal care product, cosmetics, medical product, drug, industrial product, agricultural product, and the like.

In some other embodiments, this invention relates to an emulsifier described herein, said emulsifier provides protection to the oil phase of an emulsion. Said protection is to protect the oil phase or compounds in the oil phase against photo instability, oxidation, chemical instability, volatility, pH instability, temperature instability, color instability, taste change, and flavor change, etc.

In this invention, a process is described to prepare an emulsifier from a flour, a meal, a fraction, or a whole grain of a cereal grain, a legume, a tuber, a root, a stem, a seed, a nut, or other plant material, or a combination thereof. Wherein said emulsifier comprises at least a protein component and a carbohydrate component including starch, and wherein the crystallinity of starch is less than 90% of the crystallinity of starch in the original plant material.

In some illustrative embodiments, this invention relates to an emulsion containing any of the emulsifiers described herein.

In some other embodiments, this invention relates to an emulsion that contains a flour emulsifier made from wheat, corn, rice, barley, sorghum, millet, oats, rye, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taco, tuber, tapioca, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, or the combination thereof.

In some other embodiments, this invention relates to an emulsion containing a flour emulsifier that contains at least a protein component and a carbohydrate component including starch of reduced crystallinity.

In some other embodiments, this invention relates to an emulsion containing a flour emulsifier that contains at least a protein component and a carbohydrate component including starch, wherein said emulsifier contains about 1% to about 85% protein, about 15% to about 99% carbohydrate including starch, wherein the crystallinity of starch is less than 90% of the crystallinity of the starch in its original plant material.

In some other embodiments, this invention relates to an emulsion containing a flour emulsifier that contains at least a protein component and a carbohydrate component including starch, wherein said emulsifier contains about 1% to about 85% protein, about 15% to about 99% carbohydrate including starch, wherein the crystallinity of starch is less than 85% of the crystallinity of the starch in its original plant material.

In some other embodiments, this invention related to an emulsion containing a flour emulsifier disclosed herein, said emulsion is further processed to a product, and said emulsion or product further contains other ingredients for food, beverage, personal care, drug, medical, agricultural, industrial use.

In some embodiments, this invention related to a product containing a flour emulsifier disclosed herein, said product is an oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, marijuana or cannabis containing products allowed by law, animal feed, feed additive, pet food, fish feed, fragrance, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, fabric or leather treatment chemical, plastic, rubber, container, utensil, packaging, tire, cloth, fabric, leather and the like, or the ingredient or the combination of any of above products.

In some illustrative embodiments, this invention relates to a process for preparing an emulsifier from a plant material selected from a flour, a meal, a fraction, or a whole grain of a cereal grain, a legume, a tuber, a root, a stem, a nut, a seed, or other plant material, or a combination thereof, comprising a plurality of steps of milling and heating of said plant material to afford an emulsifier, wherein said emulsifier comprises at least a protein component and a carbohydrate component.

In some other embodiments, this invention relates to a process for preparing an emulsifier, wherein the protein component is from about 1% to about 85% of said emulsifier.

In some other embodiments, this invention relates to a process for preparing an emulsifier, wherein the carbohydrate component is from about 15% to about 99% of said emulsifier.

In some other embodiments, this invention relates to a process for preparing an emulsifier, wherein the crystallinity of the starch component of said emulsifier ranges from about 0% to about 70%.

In this invention, a process is described to prepare a flour emulsifier from a plant material selected from a flour, a meal, a fraction, or a whole grain of a cereal grain, a legume, a tuber, a root, a stem, a seed, a nut, or other plant materials, or a combination thereof. The process comprises one or a plurality of steps of milling and heating of said plant material to afford an emulsifier, wherein said emulsifier comprises about 1% to about 85% of protein and 15% to about 99% of carbohydrate.

In this invention, a process is described to prepare a flour emulsifier from a plant material selected from a flour, a meal, a fraction, or a whole grain of a cereal grain, a legume, a tuber, a root, a stem, a seed, a nut, or other plant material, or a combination thereof. The process comprises one or a plurality of steps of milling and heating of said plant material to afford an emulsifier, wherein said emulsifier comprises about 1% to about 85% of protein and 15% to about 99% of carbohydrate including starch, and wherein the crystallinity of starch is less than 90% of the crystallinity of starch in the original plant material.

In some other embodiments, this invention relates to a process for preparing an emulsifier, wherein said milling process comprises at least one form of milling using hammer mill, ball mill, jet mill, stone mill, roller mill, stirred mill, stirred ball mill, colloidal mill, attritor, homogenizer, fluidizer, high speed blender, sigma blender, or extruder.

In some other embodiments, this invention relates to a process for preparing an emulsifier, wherein said process comprises at least one step of milling process with a power input rate of at least 0.05 kw per kilogram of plant material processed.

In some other embodiments, this invention relates to a process for preparing an emulsifier, wherein said milling process needs an energy input rate of at least 0.1 kw per kilogram of plant material processed.

In some other embodiments, this invention relates to a process for preparing an emulsifier, wherein said milling process needs an energy input rate of at least 0.2 kw per kilogram of plant material processed.

In some other embodiments, this invention relates to a process for preparing an emulsifier, wherein said milling process needs an energy input rate of at least 0.3 kw per kilogram of plant material processed.

In some other embodiments, this invention relates to a process for preparing an emulsifier, wherein said milling process needs an energy input rate of at least 0.4 kw per kilogram of plant material processed.

In some other embodiments, this invention relates to a process for preparing an emulsifier, wherein said milling process needs an energy input rate of at least 0.5 kw per kilogram of plant material processed.

In some other embodiments, this invention relates to a process for preparing an emulsifier, wherein said milling process needs an energy input rate of at least 0.6 kw per kilogram of plant material processed.

In some other embodiments, this invention relates to a process for preparing an emulsifier, wherein said milling process needs a total milling time of at least 1 min at said energy input rate.

In some other embodiments, this invention relates to a process for preparing an emulsifier, wherein said milling process needs a total milling time of at least 2 min at said energy input rate.

In some other embodiments, this invention relates to a process for preparing an emulsifier, wherein said milling process lasts about 2 minutes to about 50 hours.

In some other embodiments, this invention relates to a process for preparing an emulsifier, wherein said heating process comprises at least one method or facility of heating selected from vacuum oven, ventilated oven, microwave oven, near infrared oven, steaming, hot gas heating, container with jacket for heating, static heating, stirred heating, jet cooking, temperature regulator or controller, or heat exchanger.

In some other embodiments, this invention relates to a process for preparing an emulsifier, wherein said heating has a temperature from about 40° C. to about 300° C., or a stepwise gradient thereof, or a combination of different temperature thereof, for a period of about 2 min to about 100 hours.

In some other embodiments, this invention relates to a process for preparing an emulsifier, wherein said flour, meal, fraction, or a whole grain is a type or a mixture of different plant materials.

In some other embodiments, this invention relates to a process for preparing an emulsifier, wherein said milling and heating take no particular order.

In some other embodiments, this invention relates to a process for preparing an emulsifier, wherein said milling and heating are combined into a single step of milling under elevated temperature.

In some other embodiments, this invention relates to a process for preparing an emulsifier using cereal grains, wherein said cereal grains comprises one or more cereal grains from maize (corn), wheat, rice, wild rice, barley, oats, sorghum, rye, and millet etc.

In some other embodiments, this invention relates to a process for preparing an emulsifier using legumes or pulses, wherein said legumes or pulses comprise one or more from kidney bean, pinto bean, mung bean, northern bean, and peas, lentils, etc.

In some other embodiments, this invention relates to a process for preparing an emulsifier using plant materials, wherein said plant materials comprise one or more from wheat, corn, rice, barley, sorghum, millet, oats, rye, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taco, tuber, tapioca, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, or the combination thereof.

In some other embodiments, this invention relates to a process for preparing an emulsifier, wherein said process further comprising the step of hydrothermal treatment of starting material, including at least one method selected from cooking, annealing, gelatinizing, steaming, baking, microwaving, extruding, and homogenizing the plant material.

In some other embodiments, this invention relates to a process for preparing an emulsifier, wherein said process further comprising the step of drying the plant material after subjecting it to hydrothermal treatment including at least one method selected from cooking, annealing, gelatinizing, steaming, baking, microwaving, extruding, and homogenizing the plant-based material.

In some other embodiments, this invention relates to a process for preparing an emulsifier from a plant material selected from a flour, a meal, a fraction, or a whole grain of cereal grains, legumes, pulses, seeds, nuts, or other plant materials or a combination thereof, comprising one or a plurality of steps of milling and heating of said plant material to afford an emulsifier, wherein each said milling process lasts about 2 minutes to about 50 hours; wherein at least one milling process has an energy input rate over 0.05 kw per kilogram of plant material processed; wherein each said heating process has a temperature from about 40° C. to about 300° C. for a period of about 2 minutes to about 100 hours.

In some other embodiments, this invention relates to a process for preparing an emulsifier from a plant material selected from a flour, a meal, a fraction, or a whole grain of cereal grains, legumes, pulses, seeds, nuts, or other plant materials or a combination thereof, comprising one or a plurality of steps of milling and heating of said plant material to afford an emulsifier, wherein each said milling process lasts about 2 min to about 50 hours; wherein at least one milling process has an energy input rate over 0.05 kw per kilogram of plant material processed; wherein each said heating process has a temperature from about 40° C. to about 300° C. for a period of about 2 min to about 100 hours; and wherein said emulsifier contains about 1% to about 85% protein, about 15% to about 99% carbohydrate including starch; and wherein the crystallinity of said starch is less than 90% of the crystallinity of starch in the original plant materials to make said flour emulsifier.

In some other embodiments, this invention relates to an emulsifier prepared according to any of above processes disclosed herein.

In some other embodiments, this invention relates to an emulsifier prepared according to any of above processes disclosed herein. Wherein the emulsifier is able to form an emulsion with an oil phase and wherein said emulsion has emulsifier composition-to-oil ratio of about 1/100 to about 100/1, preferentially of about 1/10 to about 10/1, wherein the oil phase optionally comprises additional compounds. Wherein the emulsion is characterized as at least one of following features:
a. the average size (diameter) of the oil droplets in said emulsion is not greater than 1000 μm, 500 μm, 100 μm, 50 μm, 30 μm, 10 μm, 5 μm, 1 μm, 0.5 μm, 0.2 μm, 0.1 μm, or 0.05 μm; or
   b. the portion (by volume, weight, or number) of the oil droplets with size (diameter) greater than 500 μm is not greater than 95%, 90%, 80%, 70%, 60%, 50%, 40%, 30%, 20%, 10%, 5%, 2%, or 1%; or
   c. the portion (by volume, weight, or number) of the oil droplets with size (diameter) greater than 50 μm is not greater than 95%, 90%, 80%, 70%, 60%, 50%, 40%, 30%, 20%, 10%, 5%, 2%, or 1%; or
d. the portion (by volume, weight, or number) of the oil droplets with size (diameter) greater than 10 μm is not greater than 95%, 90%, 80%, 70%, 60%, 50%, 40%, 30%, 20%, 10%, 5%, 2%, or 1%; or
e. the portion (by volume, weight, or number) of the oil droplets with size (diameter) greater than Sum is not greater than 95%, 90%, 80%, 70%, 60%, 50%, 40%, 30%, 20%, 10%, 5%, 2%, or 1%; or
f. the portion (by volume, weight, or number) of the oil droplets with size (diameter) greater than 1 μm is not greater than 95%, 90%, 80%, 70%, 60%, 50%, 40%, 30%, 20%, 10%, 5%, 2%, or 1%; or
g. under about 1×g force (e.g. regular gravity), the visible separation in the emulsion, including but not limited to creaming, flocculation, aggregation, and precipitation, occurs at least after 0.1 min, 0.5 min, 1 min, 5 min, 10 min, 20 min, 50 min, 100 min, 200 min, 500 min, 1000 min, 2000 min, 5000 min, 10,000 min, 20,000 min, 50,000 min, or 100,000 min; or
h. after subjecting the emulsion to centrifugation for about 1 minute, the visible separation in the emulsion, including but not limited to creaming, flocculation, aggregation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g; or
i. after subjecting the emulsion to centrifugation for 10 minute, the visible separation in the emulsion, including but not limited to creaming, flocculation, aggregation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g.
j. The emulsion containing said emulsifier composition is at least 1 time, 2 times, 3 times, 4 times, 5 times, 6 times, 7 times, 8 times, 9 times, or 10 times more stable than the emulsion containing same amount of regular flour from the same plant origin.

In some other embodiments, this invention relates to an emulsifier prepared according to the process disclosed herein, wherein said emulsifier is from a flour, a meal, a fraction, or a whole grain of cereal grains, legumes, pulses, seeds, nuts, or other plant materials or a combination thereof, comprising one or a plurality of steps of milling and heating said flour, meal, fraction, or whole grain to afford an emulsifier, wherein said emulsifier contains at least a protein component and a carbohydrate component including starch, wherein the crystallinity of said starch is less than 90% of the crystallinity of starch in the original plant material.

In some other embodiments, this invention relates to an emulsifier prepared according to the process disclosed herein, wherein said protein component is from about 1% to about 85% of said emulsifier.

In some other embodiments, this invention relates to an emulsifier prepared according to the process disclosed herein, wherein said carbohydrate component ranges from about 15% to about 99% of said emulsifier.

In some other embodiments, this invention relates to an emulsifier prepared according to the process disclosed herein, wherein crystallinity of said starch component ranges from about 0% to about 70%.

In some other embodiments, this invention relates to an emulsifier prepared according to the process disclosed herein, wherein said milling process comprise at least one from hammer mill, ball mill, jet mill, stone mill, roller mill, stirred mill, stirred ball mill, colloidal mill, attritor, homogenizer, fluidizer, high speed blender, sigma blender, and extruder.

In some other embodiments, this invention relates to an emulsifier prepared according to the process disclosed herein, wherein at least one said milling process has an energy input rate more than 0.05 kw per kilogram of emulsifier processed.

In some other embodiments, this invention relates to an emulsifier prepared according to the process disclosed herein, wherein said milling processes lasts about 2 minutes to about 50 hours.

In some other embodiments, this invention relates to an emulsifier prepared according to the process disclosed herein, wherein said heating process comprise the use of at least one method or facility selected from vacuum oven, ventilated oven, microwave oven, near infrared oven, steaming, hot gas heating, container with jacket for heating, static heating, stirred heating, jet cooking, temperature regulator or controller, and heat exchanger.

In some other embodiments, this invention relates to an emulsifier prepared according to the process disclosed herein, wherein said heating has a temperature from about 40° C. to about 300° C., or a stepwise gradient thereof, or a combination of different temperature thereof, for a period of about 2 min to about 100 hours.

In some other embodiments, this invention relates to an emulsifier prepared according to the process disclosed herein, wherein said flour, meal, fraction, or whole grain is a mixture of different plant materials.

In some other embodiments, this invention relates to an emulsifier prepared according to the process disclosed herein, wherein said milling and heating take no particular order.

In some other embodiments, this invention relates to an emulsifier prepared according to the process disclosed herein, wherein said milling and heating are combined into a single step of milling under elevated temperature.

In some other embodiments, this invention relates to an emulsifier prepared according to the process disclosed herein, wherein said cereal grains comprises one or more cereals from maize (corn), wheat, rice, barley, oats, sorghum, rye, and millet etc.

In some other embodiments, this invention relates to an emulsifier prepared according to the process disclosed herein, wherein said legumes or pulses comprise one or more from kidney bean, pinto bean, mung bean, northern bean, and peas, lentils, etc.

In some other embodiments, this invention relates to an emulsifier prepared according to the process disclosed herein, wherein the process further comprises a step of hydrothermal treatment of starting material, including at least one method selected from cooking, annealing, gelatinizing, steaming, baking, microwaving, extruding, and homogenizing the plant material.

In some other embodiments, this invention relates to an emulsifier prepared according to the process disclosed herein, wherein the process further comprises a step of drying the plant material after subjecting it to hydrothermal treatment including at least one method selected from cooking, annealing, gelatinizing, steaming, baking, microwaving, extruding, and homogenizing the plant-based material.

In some other embodiments, this invention relates to an emulsifier prepared according to the process disclosed herein, wherein said emulsifier is prepared from a flour, a meal, a fraction, or a whole grain of cereal grains, legumes, seeds, nuts, or other plant materials or a combination thereof, wherein said process comprising one or a plurality of steps of milling and heating of said flour, meal, fraction, or whole grain to afford an emulsifier, wherein each said milling process lasts about 2 min to about 50 hours; wherein at least one milling process has an energy input rate of over 0.05 kw per kilogram of plant material processed; wherein said heating process has a temperature from about 40° C. to about 300° C. for a period of about 2 min to about 100 hours.

In some other embodiments, this invention relates to an emulsifier prepared according to the process disclosed herein, wherein said emulsifier is prepared from a flour, a meal, a fraction, or a whole grain of cereal grains, legumes, seeds, nuts, or other plant materials or a combination thereof, wherein said process comprising one or a plurality of steps of milling and heating of said flour, meal, fraction, or whole grain to afford an emulsifier, wherein each said milling process lasts about 2 min to about 50 hours; wherein at least one milling process has an energy input rate of over 0.1 kw, 0.2 kw, 0.3 kw, 0.4 kw, or 0.5 kw per kilogram of plant material processed; wherein said heating process has a temperature from about 40° C. to about 300° C. for a period of about 2 min to about 100 hours.

In some other embodiments, this invention relates to an emulsifier prepared according to the process disclosed herein, wherein said emulsifier is prepared from a flour, a meal, a fraction, or a whole grain of cereal grains, legumes, seeds, nuts, or other plant materials or a combination thereof, wherein said process comprising one or a plurality of steps of milling and heating of said flour, meal, fraction, or whole grain to afford an emulsifier, wherein each said milling process lasts about 2 min to about 50 hours; wherein at least one milling process has an energy input rate of over 0.05 kw per kilogram of plant material processed; wherein said heating process has a temperature from about 40° C. to about 300° C. for a period of about 2 min to about 100 hours; and wherein said emulsifier contains about 1%-85% protein, 15%-99% carbohydrate including starch; and wherein the crystallinity of starch is less than 90% of the crystallinity of starch in the original plant material. Wherein said emulsifier is able to form an emulsion with one or more feature below:
a. the average size (diameter) of the oil droplets in said emulsion is not greater than 1000 μm, 500 μm, 100 μm, 50 μm, 30 μm, 10 μm, 5 μm, 1 μm, 0.5 μm, 0.2 μm, 0.1 μm, or 0.05 μm; or
b. the portion (by volume, weight, or number) of the oil droplets with size (diameter) greater than 500 μm is not greater than 95%, 90%, 80%, 70%, 60%, 50%, 40%, 30%, 20%, 10%, 5%, 2%, or 1%; or
c. the portion (by volume, weight, or number) of the oil droplets with size (diameter) greater than 50 μm is not greater than 95%, 90%, 80%, 70%, 60%, 50%, 40%, 30%, 20%, 10%, 5%, 2%, or 1%; or
d. the portion (by volume, weight, or number) of the oil droplets with size (diameter) greater than Sum is not greater than 95%, 90%, 80%, 70%, 60%, 50%, 40%, 30%, 20%, 10%, 5%, 2%, or 1%; or
e. the portion (by volume, weight, or number) of the oil droplets with size (diameter) greater than 1 μm is not greater than 95%, 90%, 80%, 70%, 60%, 50%, 40%, 30%, 20%, 10%, 5%, 2%, or 1%; or
f. under 1×g force (e.g. regular gravity), the visible separation in the emulsion, including but not limited to creaming, flocculation, aggregation, and precipitation, occurs at least after 0.1 min, 0.5 min, 1 min, 5 min, 10 min, 20 min, 50 min, 100 min, 200 min, 500 min, 1000 min, 2000 min, 5000 min, 10,000 min, 20,000 min, 50,000 min, or 100,000 min; or
g. after subjecting the emulsion to centrifugation for about 1 minute, the visible separation in the emulsion, including but not limited to creaming, flocculation, aggregation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g; or
h. after subjecting the emulsion to centrifugation for 10 minute, the visible separation in the emulsion, including but not limited to creaming, flocculation, aggregation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g.
i. The emulsion containing said emulsifier composition is at least 1 time, 2 times, 3 times, 4 times, 5 times, 6 times, 7 times, 8 times, 9 times, or 10 times more stable than the emulsion containing same amount of regular flour from the same plant origin.

In some other embodiments, this invention relates to an emulsion that containing any of the flour emulsifier disclosed herein.

In some other embodiments, this invention relates to an emulsion that containing a flour emulsifier that is a flour from wheat, corn, rice, barley, sorghum, millet, oats, rye, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taco, tuber, tapioca, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, or the combination thereof.

In some other embodiments, this invention relates to an emulsion containing a flour emulsifier that contains at least a protein component and a carbohydrate component including starch of much reduced crystallinity.

In some other embodiments, this invention relates to an emulsion prepared according to a process comprising the steps of but not limited to.
a. preparing a lipophilic compound or a combination of lipophilic compounds thereof;
b. preparing water or an aqueous solution;
c. preparing an emulsifier or a combination thereof, wherein said emulsifier is prepared from a flour, a meal, a fraction, or a whole grain of cereal grains, legumes (pulses), or other plant materials or a combination thereof, by one or a plurality of steps of milling and heating of said flour, meal, fraction, or whole grain to afford said emulsifier, wherein said emulsifier contains at least a protein component and a carbohydrate component including starch of much reduced crystallinity;
d. mixing components of a., b. and c. to afford a mixture; and wherein the preparing and mixing take no particular order
e. homogenizing said mixture of step d. to afford an emulsion.

In some other embodiments, this invention relates to an emulsion containing an emulsifier prepared according to the process described herein, wherein said lipophilic compound in said emulsion contains a fat, oil, active pharmaceutical ingredient, herbicide, pesticide, biocide, flavor, color, natural extract, nutrient, vitamin, emollient, food additive or ingredient, cosmetic additive or ingredient, or additive or ingredient for personal care products, or a combination thereof.

In some other embodiments, this invention relates to an emulsion containing an emulsifier processed according to the process disclosed herein, wherein said aqueous solution in said emulsion is a solution, suspension, or a mixture of sugar, salt, protein, peptide, flavor, color, seasoning, vitamin, antioxidant, antimicrobial compound, antibody, enzyme, active pharmaceutical ingredient, herbicide, pesticide, nutrient, food component, food ingredient, food additive, cosmetic additive and ingredients, stabilizer, and emulsifier, moisturizer, additive or ingredient for personal care products.

In some other embodiments, this invention relates to an emulsion containing an emulsifier prepared according to the process disclosed herein, wherein said emulsion is further processed to food product, beverage, flavor additive, color additive, nutrition additive, supplement, fragrance, pharmaceutical product for human or animal consumption, product for agricultural industry, or product for personal care or hygiene, including cream, lotion, skincare products, foundation, powders, shampoo, conditioner, soap, detergent, dish cleanser, house cleaning supply, and the like.

In some other embodiments, this invention relates to an emulsion containing an emulsifier prepared according to the process disclosed herein, wherein said emulsion is further processed to food product, wherein said food product is cake, muffin, donut, tortilla, bread, flat bread, chip, cracker, cookie, pie, bar, batter, dough, icing, topping, filling, baked goods, frozen dough, ice cream, frozen food, frozen dessert, condiment or culinary food, soup, sauce, dressing, gravy, food entry, coffee creamer, dried or liquid color formulation, dried or liquid flavor formulation, dried or liquid nutrients formulation, meat product, plant meat alternative products, brine, dried powder food or animal feed, dairy, milk alternative, protein drink, energy drink, beverage, yoghurt, meal replacer, plant protein drink, etc.

In some other embodiments, this invention relates to an emulsion containing a flour emulsifier, wherein said emulsifier contains 1% to about 85% (w/w) of protein component.

In some other embodiments, this invention relates to an emulsion containing a flour emulsifier, wherein said emulsifier contains carbohydrate component ranging from about 15% to about 99%.

In some other embodiments, this invention relates to an emulsion containing a flour emulsifier, wherein the crystallinity of the starch component in said emulsifier ranges from about 0% to about 70%.

In some other embodiments, this invention relates to an emulsion containing a flour emulsifier, wherein said emulsifier was made by a process including a milling process comprise at least one selected from hammer mill, ball mill, jet mill, stone mill, roller mill, stirred mill, stirred ball mill, colloidal mill, attritor, homogenizer, fluidizer, high speed blender, sigma blender, and extruder.

In some other embodiments, this invention relates to an emulsion containing a flour emulsifier, wherein said emulsifier was made by a process including a milling process lasts about 2 minutes to about 50 hours, wherein at least one of said milling processes has an energy input rate over 0.05 kw per kilogram of emulsifier processed.

In some other embodiments, this invention relates to an emulsion containing a flour emulsifier, wherein said emulsifier was made by a process including a heating process comprise the use of at least one method or facility selected from vacuum oven, ventilated oven, microwave oven, near infrared oven, steaming, hot gas heating, container with jacket for heating, static heating, stirred heating, jet cooking, temperature regulator or controller, and heat exchanger.

In some other embodiments, this invention relates to an emulsion containing a flour emulsifier, wherein said emulsifier was made by a process including a heating process, wherein said heating has a temperature from about 40° C. to about 300° C., or a stepwise gradient thereof, or a combination of different temperature thereof, for a period of about 2 min to about 100 hours.

In some other embodiments, this invention relates to an emulsion containing a flour emulsifier, wherein said flour is a mixture of different plant materials, including but not limited to cereal, legume, pulse, root, tuber, stem, seed, nut.

In some other embodiments, this invention relates to an emulsion containing a flour emulsifier, wherein said emulsifier was made by a process including a milling process and a heating process, wherein said milling and heating take no particular order.

In some other embodiments, this invention relates to an emulsion containing a flour emulsifier, wherein said emulsifier was made by a process including a milling process and a heating process, wherein said milling and heating are combined into a single step of milling under an elevated temperature.

In some other embodiments, this invention relates to an emulsion containing a flour emulsifier, wherein said cereal grains comprise one or more cereals from maize (corn), wheat, rice, barley, oats, sorghum, rye, and millet etc.

In some other embodiments, this invention relates to an emulsion containing a flour emulsifier, wherein said flour is made from legumes or pulses comprise kidney bean, pinto bean, mung bean, northern bean, and peas, etc.

In some other embodiments, this invention relates to an emulsion containing a flour emulsifier, wherein said flour is made from a flour, meal, fraction, or whole grain is of corn, wheat, rice, oats, rye, barley, sorghum, amaranth, millet, legumes, beans, peas, peanut, soybean, potato, yam, and tapioca, or a combination thereof.

In some other embodiments, this invention relates to an emulsion prepared according to the process disclosed herein, wherein said process further comprises the step of hydrothermal treatment of starting material, including at least one method selected from cooking, annealing, gelatinizing, steaming, baking, microwaving, extruding, and homogenizing the plant material.

In some other embodiments, this invention relates to an emulsion prepared according to the process disclosed herein, wherein said process further comprises the step of drying the plant material after subjecting it to hydrothermal treatment including at least one method selected from cooking, annealing, gelatinizing, steaming, baking, microwaving, extruding, and homogenizing the plant-based material.

In some other embodiments, this invention relates to an emulsion containing an emulsifier prepared according to the process disclosed herein, wherein said emulsion using an emulsifier prepared from a flour, a meal, a fraction, or a whole grain of cereal grains, legumes, or other plant materials or a combination thereof, comprising one or a plurality of steps of milling and heating of said flour, meal, fraction, or whole grain to afford said emulsifier,
wherein each said milling process lasts about 2 min to about 50 hours;
wherein at least one of the milling processes has power input of at least 0.05 kw per kg of plant material processed;
wherein said heating process has a temperature from about 40° C. to about 300° C. for a period of about 2 min to about 100 hours

In some other embodiments, this invention relates to an emulsion containing an emulsifier prepared according to the process disclosed herein, wherein said emulsion using an emulsifier prepared from a flour, a meal, a fraction, or a whole grain of cereal grains, legumes, or other plant materials or a combination thereof, comprising one or a plurality of steps of milling and heating of said flour, meal, fraction, or whole grain to afford said emulsifier,
wherein each said milling process lasts about 2 min to about 50 hours;
wherein at least one of said milling process has a power input or power consumption of not less than 0.05 kw per kg of plant material processed;
wherein said heating process has a temperature from about 40° C. to about 300° C. for a period of about 2 min to about 100 hours; and
wherein said emulsifier contains about 1%-85% protein, 15%-99% carbohydrate including starch with a crystallinity less than 90% of its original value.

In some embodiments, the heating or heating process of making an emulsifier is conducted at a temperature of 40° C. to 300° C. for 2 minutes to 100 hours.

In one embodiment, the heating is conducted at a temperature of 50° C.-200° C. for 10 minutes to 20 hours.

In another embodiment, the heating is conducted at a temperature of 80° C.-150° C. for 20 minutes to 10 hours.

In another embodiment, the heating is conducted at a temperature of 90° C.-130° C.

In another embodiment, the heating is conducted at a temperature of 100° C.-130° C.

In another embodiment, the heating is conducted for 20 minutes to 5 hours.

In another embodiment, the heating is conducted for 30 minutes to 3 hours.

In this invention, the milling or milling process is conducted for 2 minutes to 50 hours with a power input (or powder consumption) of the milling unit of not less than 0.05 kw (kilowatts).

In one embodiment, the milling is conducted for 2 minutes to 20 hours with a power input (or powder consumption) of the milling unit of not less than 0.10 kw.

In another embodiment, the milling is conducted for 5 minutes to 10 hours.

In another embodiment, the milling is conducted for 5 minutes to 5 hours.

In another embodiment, the milling is conducted for 10 minutes to 5 hours.

In another embodiment, the milling is conducted with a power input (or powder consumption) of the milling unit of not less than 0.15 kw.

In another embodiment, the milling is conducted with a power input (or powder consumption) of the milling unit of not less than 0.2 kw.

In another embodiment, the milling is conducted with a power input (or powder consumption) of the milling unit of not less than 0.3 kw.

In another embodiment, the milling is conducted with a power input (or powder consumption) of the milling unit of not less than 0.4 kw.

In another embodiment, the milling is conducted with a power input (or powder consumption) of the milling unit of not less than 0.5 kw.

In another embodiment, the milling is conducted with a power input (or powder consumption) of the milling unit of not less than 0.6 kw.

In another embodiment, the milling is conducted with a power input (or powder consumption) of the milling unit of not less than 0.8 kw.

In another embodiment, the milling is conducted with a power input (or powder consumption) of the milling unit of not less than 0.9 kw.

In another embodiment, the milling is conducted with a power input (or powder consumption) of the milling unit of not less than 1 kw.

In another embodiment, the milling is conducted with a power input (or powder consumption) of the milling unit of not less than 1.2 kw.

In another embodiment, the milling is conducted with a power input (or powder consumption) of the milling unit of not less than 1.5 kw.

In another embodiment, the milling is conducted with a power input (or powder consumption) of the milling unit of not less than 2 kw.

In another embodiment, the milling is conducted with a power input (or powder consumption) of the milling unit of not less than 3 kw.

In another embodiment, the milling is conducted with a power input (or powder consumption) of the milling unit of not less than 5 kw.

In another embodiment, the milling is conducted with a power input (or powder consumption) of the milling unit of not less than 6 kw.

In another embodiment, the milling is conducted for 20 minutes to 5 hours with a power input (or powder consumption) of the milling unit of not less than 0.2 kw.

In another embodiment, the milling is conducted for 20 minutes to 3 hours with a power input (or powder consumption) of the milling unit of not less than 0.3 kw.

In another embodiment, the milling is conducted for 20 minutes to 3 hours with a power input (or powder consumption) of the milling unit of not less than 0.4 kw.

In some other embodiments, this invention relates to an encapsulation composition containing an emulsifier disclosed herein.

In some other embodiments, this invention relates to an encapsulation composition containing an emulsifier, wherein said emulsifier is prepared from a flour, a meal, a fraction, or a whole grain of cereal grains, legumes, tubers, roots, stems, seeds, nuts, or other plant materials or a combination thereof, by one or a plurality of steps of milling and heating of said flour, meal, fraction, or whole grain to afford said emulsifier, wherein said emulsifier contains at least a protein component and a carbohydrate component including starch of much reduced crystallinity.

In some other embodiments, this invention relates to an encapsulation composition further containing a lipophilic compound or a combination of lipophilic compounds thereof;

In some other embodiments, this invention relates to an encapsulation composition disclosed herein, wherein said lipophilic compound is an active pharmaceutical ingredient, herbicide, pesticide, flavor, nutrient, food additive or ingredient, cosmetic additive or ingredient, or additive or ingredient for personal care products.

In some other embodiments, this invention relates to an encapsulation composition disclosed herein, wherein said aqueous solution is a solution of sugar, salt, protein, peptide, flavor, color, vitamin, antioxidant, antimicrobial compound, antibody, enzyme, active pharmaceutical ingredient, herbicide, pesticide, flavor, nutrient, food additive or ingredient, cosmetic additive or ingredients, stabilizer, and/or emulsifier.

In some other embodiments, this invention relates to an encapsulation composition disclosed herein, wherein said encapsulation composition is further processed to or used in food product, beverage, fragrance, pharmaceutical product for human or animal consumption, product for agricultural industry, product for personal hygiene, lotion, shampoo, conditioner, soap, and/or the like.

In some other embodiments, this invention relates to an encapsulation composition disclosed herein, wherein said food product is cake, muffin, donut, tortilla, bread, flat bread, chip, cracker, baked good, ice cream, soup, sauce, dressing, soup, culinary food, meant products, brine, gravy, coffee creamer, dried powder food or animal feed.

In some other embodiments, this invention relates to an encapsulation composition disclosed herein, wherein said emulsifier is prepared from a flour, meal, fraction, or whole grain is of wheat, corn, rice, wild rice, barley, fonio, Job's tears, sorghum, millet, oats, rye, teff, triticale, buckwheat, tartary buckwheat, amaranth, quinoa, pitseed goosefoot, canihua, chia, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taro, tuber, cassava (tapioca), water chestnut, arrowroot, sweet potato, Chinese yam, lotus root, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, or the combination thereof.

In some other embodiments, this invention relates to an encapsulation composition disclosed herein, wherein the process of making said encapsulation further comprises the step of hydrothermal treatment of starting plant material, including at least one method selected from cooking, annealing, gelatinizing, steaming, baking, microwaving, extruding, and homogenizing the plant material.

In some other embodiments, this invention relates to an encapsulation composition disclosed herein, wherein the process of making said encapsulation further comprises a step of drying the plant material after subjecting it to hydrothermal treatment including at least one method selected from cooking, annealing, gelatinizing, steaming, baking, microwaving, extruding, and homogenizing the plant-based material.

In some other embodiments, this invention relates to an encapsulation composition using an emulsifier prepared from a flour, a meal, a fraction, or a whole grain of cereal grains, legumes, or other plant materials or a combination thereof, comprising one or a plurality of steps of milling and heating of said flour, meal, fraction, or whole grain to afford said emulsifier,
wherein each said milling process lasts about 2 min to about 50 hours;
wherein at least one of said milling process has a power input of over 0.05 kw per kg of plant material processed;
wherein said heating process has a temperature from about 40° C. to about 300° C. for a period of about 2 min to about 100 hours; and
wherein said emulsifier contains about 1%-85% protein, 15%-99% carbohydrate including starch, and
wherein the crystallinity of said starch is less than 90% of the crystallinity of starch in the original plant material.

In some other embodiments, this invention relates to an emulsifier prepared by alternating milling and heating of a plant material, comprising at least a protein component ranging from about 1%-85% of the emulsifier and a carbohydrate component ranging from about 15%-99% of the emulsifier, wherein said carbohydrate component comprising starch, and wherein the crystallinity of starch is less than 90% of the crystallinity of starch in the original plant material.

In some other embodiments, this invention relates to an emulsion comprising a lipophilic compound, an emulsifier prepared according to the process disclosed herein, and an aqueous solution mixed by homogenization, wherein said emulsion has at least twice stability compared to the homogenized lipophilic compound in said aqueous solution without said emulsifier prepared according to the process disclosed herein.

In some other embodiments, this invention relates to an encapsulation composition disclosed herein, wherein the process of making said encapsulation further comprises a step of dehydrating.

Lipophilic materials, which include oil, fat, oil or fat-soluble compounds, and their combinations, have been used broadly in the food, personal care, pharmaceutics, agricultural chemical, and other areas. A primary method of formulating and/or using lipophilic materials is to form emulsions, so they can be water-soluble or dispersible, or mixable with other components in formulations. In general, emulsions can be oil-in-water, water-in-oil, or a combination of both.

For food, emulsion systems include oil-containing beverages, dairy products, ice cream, meat products, bakery, sauces, soups, salad dressing, gravies, formulations of oleoresins and essential oils for coloring, flavoring, nutrient, and providing protection from oxidations, encapsulation and microencapsulation of active ingredients, coffee creamers, and the delivery of lipophilic nutrients such as omega-3 fatty acids (e.g. fish oil, DHA, EPA). Not only a lot of food beverage are emulsions systems, but also, emulsions are formed over the processing for making certain dried food and food ingredients. For examples, encapsulated fat or oil-soluble flavors are made by forming oil-in-water emulsions first, and then the emulsions are spray-dried into powder solid. For personal care, a lot of products are emulsions, such as creams, lotions, shampoos, soaps, etc. In personal care applications, emulsions are prepared to deliver antioxidants such as vitamin A and vitamin E by forming emulsions first with active ingredients, and then add the emulsions to the end products. For pharmaceutics, the emulsions are used to solubilize and deliver lipophilic or poorly water-soluble pharmaceutical active ingredients. For agricultural chemicals, emulsions are used to disperse or solubilize pesticides, plant nutrients, hormones, or other active ingredients. In addition, the emulsions are used in many other areas not listed here.

A bakery food, including but not limited to cake, muffin, donut, cookie, bread, flat bread, pie, cracker, chip, tortilla, pudding, bar, dessert, icing, topping, filling, candy, frozen dessert, refrigerated or frozen dough, snack food, containing a flour emulsifier disclosed in this invention. Said bakery food may also contain other emulsifiers, stabilizers, sugar, butter, oil, butter, gums, protein, color, flavor, egg, starch, etc.

A food ingredient or food component, including but not limited to color formulation, flavor formulation, natural extract formulation, antioxidants formulation, nutrient formulation, DHA formulation, vitamin formulation, micro nutrients additives, dietary supplement, supplement ingredient, bakery ingredients mix, beverage ingredients mix, containing a flour emulsifier disclosed in this invention. Said food ingredient or food component may further contain other emulsifiers, active ingredients, nutrients, stabilizers, flavors, colors, proteins, starch, fat or oil, gums, etc.

A frozen food, including frozen entries, dairy or non-dairy ice cream, frozen yoghurt, frozen dessert, containing a flour emulsifier disclosed in this invention. Said frozen food may also contain other food ingredients, emulsifiers, stabilizers, etc. Said frozen food may have improved freeze and thaw stability, better texture, better mouthfeel, and taste.

A condiment or culinary food, including but not limited to sauce, dressing, soup, entries, containing a flour emulsifier disclosed in this invention. Said condiment may also contain other emulsifiers, stabilizers, flavor, seasoning, etc.

A beverage, including but not limited to protein drink, energy drink, meal replacer, dried beverage powder, dairy and non-dairy beverage, almond milk, nut milk, soy milk, yoghurt, probiotic or prebiotic drink, nutrition supplement, containing a flour emulsifier disclosed in this invention. Said beverage may also contain other emulsifiers, stabilizers, flavor, other food ingredients, etc.

A meat product, including but not limited to sausage, deli meat, canned meat, meat brine, meat alternative, plant sourced protein, containing a flour emulsifier disclosed in this invention. Said meat or protein product may also contain other emulsifiers, stabilizers, flavor, other food ingredients, etc.

A feed product, including animal feed, feed additives, pet food, fish feed, containing a flour emulsifier disclosed in this invention.

A personal care product, including cream, lotion, moisturizer, skincare product, cosmetics, powder, foundation, eye shadow, bronzer, makeup, cleanser, soap, serum, sunscreen, shampoo, conditioner, hair product, detergent, dishwasher, wipe, baby powder, ointment, balm, lip product, household spray, fabric spray, fabric coating, containing a flour emulsifier disclosed in this invention. Said personal care product may also contain other emulsifiers, stabilizers, gums, surfactants, emollient, moisturizing agent or polysaccharides, sunscreen active ingredients, antioxidants, vitamins, skin nutrients, color, oil, wax, skin active compounds, etc.

A pharmaceutical product, including drug, antibiotics, anti-infection drug, anti-viral drug, anti-fungal drug, anti-cancer drug, vaccine, steroid, nasal spray, topical cream, ointment, containing a flour emulsifier disclosed in this invention. Said pharmaceutical product may also contain active pharmaceutical ingredients, excipients, etc.

An agriculture product, including pesticide, herbicide, plant protection, plant nutrients, fertilizer, spray, plant hormone, seed protection, biocides, seed coating, fungicides, containing a flour emulsifier disclosed in this invention. Said agriculture product may also contain bioactive compound, biocide, agricultural active ingredients, other emulsifiers, stabilizers, etc.

An industrial product, including plastic, rubber, container, utensil, packaging, tire, cloth, fabric, film, leather, containing or treated with a formulation containing a flour emulsifier disclosed in this invention.

This invention is also related to using flour emulsifiers in above food, beverage, personal care, pharmaceutical, agricultural, and industrial products, and creating new formulations of food, beverage, personal care, pharmaceutical, agriculture, and industrial products that contain flour emulsifier. In some embodiments, we first time developed food, beverage, personal care, pharmaceutical, agriculture, and industrial products that contain flour emulsifier disclosed in this invention.

Above new food, beverage, personal care, pharmaceutical, agriculture, and industrial products described include but not limited to a oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, animal feed, feed additive, pet food, fish feed, fragrance, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, fabric or leather treatment chemical, plastic, rubber, container, utensil, packaging, tire, cloth, fabric, leather and the like, or the ingredient of any of above products.

Above new food, beverage, personal care, pharmaceutical, agriculture, and industrial products described may also include one or more other ingredients for food, beverage, personal care, pharmaceutical, medical, agricultural, and industrial use, including but not limited to protein, carbohydrate, polysaccharide, moisturizer, regular flour, vitamin, DHA, EPA, micronutrients, flavor, fragrant, color, small-molecule emulsifiers, mono/di-glycerides, polysorbates, calcium stearoyl lactylate, sodium stearoyl lactylate, polyglycerol ester, sorbitan ester, propylene glycol ester, sugar ester, acetylated monoglyceride, lactylated monoglycerides, lecithin, saponin, modified starch, gum arabic (gum acacia), protein-based emulsifiers such as pea protein, sodium caseinate, whey protein isolates, starch, maltodextrins, syrups, sugars, sugar alcohols, oligosaccharides, hydrolyzed biopolymers, polysaccharides hydrolysates, protein hydrolysates, rheological property modifiers including but not limited to polysaccharide gums, proteins, xanthan gum, locus bean gum, guar gum, alginate, pectin, cellulose, carboxymethylcellulose, modified cellulose, starch or its derivatives, protein-based hydrocolloids, gelatin, soy protein, pea protein, egg white, protein materials including but not limited to pea proteins, soy proteins, cricket powder, protein hydrolysates, whole egg, egg yolk, egg whites, pea flour, bean flours, lentil flour, prebiotics, probiotics, microbiome-related materials, and the like.

The use of emulsifiers is essential for the formation and stability of emulsions. Emulsifiers, sometimes also referred as surfactants, are a group of surface-active, amphiphilic materials that can be distributed at the oil-water interface. Usually an amphiphilic molecule or particulate has one or multiple lipophilic moieties and one or multiple hydrophilic moieties. By reducing the interfacial tension, emulsifiers enhance the stability of oil-water interface and reduce the rate of aggregations of dispersed droplets.

In addition to emulsification properties, flour emulsifier in this invention also contribute with properties including but not limited to thickening, viscosity, texture improving, creaminess, improved mouth feel, improved freeze-thaw stability, improved physical stability, and or other properties to an emulsion or a product.

The flour emulsifier in this invention also provides protection to the oil phase of an emulsion. Said protection is protecting the oil phase or compounds in the oil phase again photo instability, oxidation, chemical instability, volatility, pH instability, temperature instability, or color instability, taste change, flavor change, etc. Products containing flour emulsifier hereof have better photo stability, color stability, chemical stability, anti-oxidant property or better resisting to oxidation, better flavor and fragrance stability, pH stability, and temperature stability, etc.

Sometimes, an HLB (hydrophile-lipophile-balance) value can be used to indicate the degree of an emulsifier in its distribution in water and oil phases. Usually, an emulsifier with a high HLB value is used to stabilize oil-in-water emulsion, and an emulsifier with a low HLB value is used to stabilize water-in-oil emulsion.

In addition to HLB value, emulsifiers can be classified based on other features. For example, emulsifiers can be classified based on their size, such as small-molecule emulsifiers (e.g. lecithin, saponin, fatty acids, sugar esters, mono/diglycerides, polysorbates) and macromolecule emulsifiers (e.g. gum arabic, octenylsuccinate starch, octenylsuccinate gum arabic). In particular, particle-based emulsifiers can form Pickering emulsion, usually with high stabilities.

Emulsifiers can be classified based on their resources, i.e. synthetic, partially synthetic, and naturally occurring. Sugar esters, polysorbates, mono- and diglycerides, propylene glycol esters of fatty acids, sorbitan monostearate and tristearate, sodium and calcium stearoyl lactylate, and DATEM (diacetyl tartaric acid ester of mono- and diglycerides) are examples of synthetic emulsifiers. Gum arabic, lecithin, dairy proteins (e.g. caseinate, whey protein), and saponin are examples of natural emulsifiers. Octenylsuccinate starch (OSA-starch) and octenylsuccinate gum arabic are examples of partially synthetic emulsifier.

The primary property to evaluate an emulsion is its stability. The stability of an emulsion can be conceptually classified as physical stability and chemical stability. Physical stability is usually used to describe the change of physical state of an emulsion, which is mostly related to the state of the dispersed droplets (e.g. the oil droplets in the oil-in-water emulsions or the water droplets in the water-in-oil emulsions). In general, the physical stability of an oil-in-water emulsion is described as the extent or the capability of this emulsion against creaming, sedimentation, flocculation, coalescence, or aggregation of oil droplets. The rate of the loss of volatile lipophilic components, such as an essential oil from the emulsion, can also be considered as an indicator of physical stability.

Chemical stability of an emulsion is usually used to describe the capability of emulsion against the chemical change of any component in an emulsion. In an oil-in-water emulsion, chemical stability can be referred as the resistance of lipophilic component against light, heating, pro-oxidative compounds, radiations, or the combinations of these factors. For example, an emulsion of paprika oleoresin may be susceptible to prolonged exposure to light and thus lose its specific color strength. An emulsion of fish oil may be degraded by the oxidative components in the aqueous system such as oxygen, free radicals, and heavy metal ions, and such degradations can be accelerated in the presence of high temperature.

The stability of emulsion can be affected by multiple factors. Among those, the type of emulsifier used can be very important. In general, emulsifier molecules (or particles) form an interfacial layer, that is, a layer at the oil-water interface. The properties of interfacial layer usually have large impact on the interactions between a droplet and its surrounding components. At the interface, the hydrophilic portion of the emulsifier interacts with the aqueous phase and the lipophilic portion interacts with the oil phase. The interfacial layer not only reduces the interface tension, but also forms a physical barrier against the mass transfer between the oil and aqueous phases. For example, large molecules or nano-particulates may form a thick and/or dense interfacial layer and thus reduce the diffusion of pro-oxidative compounds from aqueous phase to oil phase, leading to reduced oxidation of lipophilic compounds. A thick interfacial layer may also benefit the physical stability of an emulsion through the steric repelling among individual droplets, thus reducing the rate of agglomeration, aggregation, flocculation, or coalescence. A thick and dense interfacial layer may also effectively reduce the evaporation loss of volatile lipophilic compounds, such as essential oils, from dispersed oil droplets. Ionized emulsifier may form an interfacial layer with charges (either positive charge or negative charge), thus causing static repelling among individual droplets and enhancing the physical stability of emulsion.

In addition to the type of emulsifier, other factors (e.g. environmental factors) may also affect emulsion stability. These factors include, but not limited to the pH value, ionic strength, viscosity, temperature change, presence of oppositely charged molecules or particles, presence of polymers or biopolymers, exposure to light, and presence of oxidative compounds. For example, an extreme pH value (very high or low) or high salt concentration could lead to flocculation of emulsions. High viscosity may stabilize an emulsion from creaming or sedimentation. High or low temperature may increase or decrease the oxidation rate. High light intensity, high temperature, or the presence of oxidative compounds may accelerate the oxidation and degradation of lipophilic compound in the oil phase. High temperature may lead to a quick loss of volatile components (e.g. essential oil) from the oil phase. Presence of polymer or biopolymer may lead to bridging flocculation or depletion flocculation.

In many circumstances, the environmental factors affect the stability of an emulsion through affecting the performance of emulsifiers. For example, at low pH, the protonation (i.e. adding a proton) to a negatively charged carboxylate group may reduce the charge density of the emulsifier, thus reducing the repelling among individual oil droplets in an oil-in-water emulsion. On the other hand, high pH may lead to higher stability of oil-in-water emulsions through bringing more negative charges.

In certain circumstances, adding a different emulsifier in an emulsion does not necessarily lead to enhanced stability. For example, adding a small-molecule emulsifier in a large-molecule emulsifier-stabilized emulsion may cause the replacement of large molecules with small molecules at the interfacial layer, which might reduce the physical stability of the emulsion.

In an oil-in-water emulsion, adding polymer or biopolymer (e.g. polysaccharides) usually increases the viscosity of an emulsion and thus reduces the rate of creaming, flocculation, coalescence, or aggregation. However, some polymer or biopolymer may lead to instability of emulsions. For example, bridging flocculation may occur if the added polymer or biopolymer has an opposite charge with the emulsifier (and thus the oil droplets). High concentration of polymer or biopolymer may lead to depletion flocculation.

In the food systems, there are several types of emulsifiers normally used: (1) small molecule surfactants, such as fatty acids, monoglyceride, saponin, and DATEM, (2) protein based emulsifiers, such as sodium caseinate and whey protein, (3) polysaccharide based emulsifiers, such as gum arabic and OSA-starch. Due to their difference in size, charge type and density, and the nature of lipophilic and hydrophilic moieties, these emulsifiers have displayed different properties in forming and stabilizing emulsions. In addition, the stability of lipophilic materials in the solids prepared from the dehydration of emulsions may also different with different types of emulsifier.

Use of emulsifiers and wall materials for encapsulations. In many circumstances, an emulsion needs to be dehydrated for convenient processing, storage, and usage. In other circumstances, a lipophilic compound, such as a polyunsaturated fatty acid (PUFA), essential oil, or natural colorant, needs to be in a solid form to be protected from oxygen, moisture, light, and other environmental factors that may cause the oxidation, degradation, and/or other chemical changes. For this purpose, an emulsion formed with the lipophilic material needs to be dehydrated to obtain solid. The mostly used dehydration process is spray drying, which yields the micron-sized particles in a process usually called encapsulation (or microencapsulation). Other methods can also be used for the encapsulation of lipophilic materials, such as freeze-drying, drum drying, extrusion, and coacervation.

In the dehydrated product of an emulsion, oil droplets are covered with emulsifiers and embedded in bulking agents, such as gelatin, maltodextrin, or starch. In the solid, these materials (e.g. emulsifiers and bulking agents) have the capability to protect oil droplets from oxygen, moisture, light, and other degradation factors, and usually they are called as “wall materials”, a normally used term in the description of encapsulation systems. Some large-molecule emulsifiers such as gum arabic, sodium caseinate, and octenylsuccinate starch, not only perform as emulsifiers during emulsification, but also function as wall materials. The molecular weight, hygroscopicity (the capability to absorb moisture from environment), glass transition temperature (Tg), and other physicochemical properties of wall materials may substantially affect the stability of encapsulated lipophilic materials against oxygen, moisture, light, and abnormal temperatures. Usually, a biopolymer with good film-forming and barrier properties is preferred as wall materials.

Usually, the process of preparing encapsulated composition including lipophilic materials includes several steps: (1) incorporating (e.g. dissolving) the lipophilic material in an oil, (2) dissolving or dispersing the emulsifier(s) and wall materials in an aqueous solvent (e.g. water or buffer), (3) mixing the oil and aqueous solvent and subject the mixture to homogenization through high-speed, high-pressure, high-shear, or ultrasonic approaches, with a goal to form micron or sub-micron sized oil droplets, and (4) subjecting the emulsion to a dehydration procedure, such as spray-drying, to collect the solid that is usually in a powder form. Sometimes, the powder is further granulated to form larger particles for better flowing and dispersing properties.

For food applications, most food-grade emulsifiers can be used to prepare emulsions for encapsulation. For wall materials, however, only those food materials with acceptable barrier properties are preferred. Biopolymers usually are good candidates for wall materials, and they include but are not limited to: (1) protein-based materials such as caseinate, whey protein, and gelatin, and (2) oligosaccharides and polysaccharides such as inulin, polydextrose, maltodextrin, gum arabic, pullulan, and cellulose derivatives.

Several biopolymers can be used as both emulsifiers and wall materials, such as sodium caseinate, gum arabic, octenylsuccinate starch, and octenylsuccinate gum arabic. These bi-functional biopolymers are unique for encapsulations; however, they are usually associated with high cost. Therefore, non-emulsifier, low-cost wall materials, such as maltodextrins or corn syrups are added to partially replace bi-functional biopolymers.

To address the problems and challenges encountered in emulsion systems, encapsulation systems, and products, we have been looking for cost-effective and sustainable emulsifiers for food, beverage, personal care, drug, medical, agricultural, industrial applications. Through assiduous effort, we have surprisingly found that high-quality emulsifiers can be prepared from starch and protein-containing flours from a variety of plant materials, such as cereals (e.g. corn, wheat, rice), legumes (beans, peas), and tubers and roots (potato and sweet potato).

In each of these flours, there is a substantial amount of starch and protein materials. However, conventional flour-making processes do not generate flours with acceptable emulsification properties. In another word, flour emulsifiers cannot be prepared using any reported or practiced milling approach prior to this invention.

For the first time, we found that prolonged milling in combination with heating was able to generate flours with strong emulsification capabilities comparable with or superior to that of gum arabic and octenylsuccinate starch (OSA-starch), two mostly used emulsifiers in the food industry.

The preparation of flour emulsifier can involve physical processing only, without adding any external chemicals such as acids, bases, salts, and enzymes. Adding external chemicals during the processing, however, may affect the functionalities of flour products either positively or negatively.

The following paragraphs list some examples of practice or embodiments only to depict the concept of this invention. The list does not limit the scope in which the invention applies.

In one embodiment, a cereal grain or legume seed is heated for a total of 2.5 hour at 110° C. and ball-milled for a total of 4 hours. The flour yielded shows strong emulsification capability.

In another embodiment, a milled rice grain is milled for 2 hours to yield a rice flour with emulsification capability greater than that of a regular rice flour.

In another embodiment, a milled rice grain is milled for 2 hours to yield a rice flour with emulsification capability greater than that of a regular rice flour, wherein the milling process has a power input of over 0.05 kw per kg of the plant material processed.

In another embodiment, the rice flour that is treated with 2 hours of milling is further heated for 1 hour at 110° C., yielding a flour with greater emulsification capability than that of the rice flour prepared without heating.

In another embodiment, a regular corn flour is heated for 2 hours at 110° C. and milled for 3 hours, yielding a corn flour with enhanced emulsification capability.

In another embodiment, mung bean seeds are ground and passed 80-mesh sieve, and thereafter subjected to 3 hours of milling and then 1.5 hours of 110° C. heating. The mung bean flour produced shows emulsification properties.

In another embodiment, soaked, steamed, and then dried split pea is ground to pass 80-mesh sieve, and thereafter subjected to 1 hour of 110° C. heating, 3 hours of milling, and then 1.5 hours of 110° C. heating. The flour thus prepared shows emulsification capability.

In another embodiment, soaked, steamed, and then dried kidney bean is ground to pass 80-mesh sieve, and thereafter subjected to a combined heating for 2.5 hours at 110° C. and ball-milling for 4 hours. The flour thus prepared shows emulsification capability.

In another embodiment, a rice grain is first ground and passed 80-mesh sieve, dried to moisture content to 0.0 to 10%, subjected to 10 minutes to 10 hours of ball-milling, and thereafter treated with 10 minutes to 20 hours of 100 to 150° C. heating. The rice flours produced show higher emulsification properties than the rice flour without being subjected to combined heating and ball-milling.

Without limiting the scope and applications of this invention, the mechanism of high emulsification capability of flour emulsifiers is probably associated with the physical state of protein and starch, such as their particle size, crystallinity, and conformation change. For example, prolonged milling or heating may lead to a reduction of starch crystallinity and a change of hydrophobic packing of protein. While the protein and starch are subjected to physical processing only, the exposure of hydrophobic portions of proteins as well as a quick dissolution of starch material may synergistically enhance the emulsification properties of flours.

Since different plant materials have different types of proteins and starches, the emulsification properties of corresponding flours can be different accordingly.

The flour emulsifiers in this invention are used to form and stabilize emulsions.

In one embodiment, a flour emulsifier is used to emulsify a lipophilic material to form an emulsion. The emulsion prepared using the flour emulsifier shows greater stability than the emulsion prepared without using the flour emulsifier.

In another embodiment, paprika oleoresin is emulsified with a rice flour emulsifier. The emulsion formed shows greater stability than the emulsion formed without the rice flour emulsifier and the emulsion formed with gum arabic.

In another embodiment, astaxanthin oleoresin is emulsified with a rice flour emulsifier. The emulsion formed shows greater stability than the emulsion formed without the rice flour emulsifier and the emulsion formed with gum arabic.

In another embodiment, a mixed tocopherol is emulsified with a rice flour emulsifier. The emulsion formed shows greater stability than the emulsion formed without the rice flour emulsifier and the emulsion formed with gum arabic.

In another embodiment, retinol oil is emulsified with a rice flour emulsifier.

The emulsion formed shows greater stability than the emulsion formed without the rice flour emulsifier and the emulsion formed with gum arabic.

In another embodiment, soybean oil is emulsified with a rice flour emulsifier.

The emulsion formed shows greater stability than the emulsion formed without the rice flour emulsifier and the emulsion formed with gum arabic.

In another embodiment, fish oil is emulsified with a rice flour emulsifier. The emulsion formed shows greater stability than the emulsion formed without the rice flour emulsifier and the emulsion formed with gum arabic.

In another embodiment, orange oil is emulsified with a rice flour emulsifier.

The emulsion formed shows greater stability than the emulsion formed without the rice flour emulsifier and the emulsion formed with gum arabic.

In another embodiment, rosemary oil is emulsified with a rice flour emulsifier. The emulsion formed shows greater stability than the emulsion formed without the rice flour emulsifier and the emulsion formed with gum arabic.

The flour emulsifiers in this invention are used for encapsulations and microencapsulations

Surprisingly, it was found that flour emulsifiers generated in this invention not only showed outstanding emulsification capabilities, but also can be used as wall materials for encapsulations or microencapsulations. While without limiting the scope of this invention, we think that flour emulsifiers can be used as the wall materials for encapsulation due to their properties as biopolymers. Flour emulsifiers contain protein and starch, both having high molecular weight, high glass transition temperature (Tg), and low hygroscopicity. Such properties of flour emulsifiers allow for sufficient solid coverage over oil droplets and therefore high protection of lipophilic compounds from oxygen, moisture, light in the environment, and other degrading factors. It was further found that the solid products of encapsulation were effectively hydrated, dispersed, or dissolved in water or other aqueous solvent, forming stable emulsions.

In one embodiment, a flour emulsifier is used to emulsify a lipophilic material to form an oil-in-water emulsion. The emulsion is thereafter dehydrated to form a solid material. The solid is thereafter successfully rehydrated into a stable emulsion.

In another embodiment, a rice flour emulsifier is used to emulsify a paprika oleoresin and form an emulsion. This emulsion is dried in a ventilated oven at 60° C. to collect solid. The solid is dispersed in water and a stable emulsion is formed.

In another embodiment, a rice flour emulsifier is used to emulsify orange oil and an emulsion is formed. The emulsion is spray-dried to collect solid in powder form. The powder is rehydrated to form a stable emulsion.

In another embodiment, a rice flour emulsifier is used to emulsify fish oil and an emulsion is formed. The emulsion is spray-dried to collect solid in powder form. The powder is rehydrated to form a stable emulsion.

In another embodiment, a rice flour emulsifier is used to emulsify rosemary oil and an emulsion is formed. The emulsion is spray-dried to collect solid in powder form. The powder is rehydrated to form a stable emulsion.

In another embodiment, a rice flour emulsifier is used to emulsify a vegetable oil and an emulsion is formed. The emulsion is spray-dried to collect solid in powder form. The powder is rehydrated to form a stable emulsion.

In another embodiment, a rice flour emulsifier is used to emulsify a vegetable oil that dissolves paprika oleoresin, and an emulsion is formed. The emulsion is spray-dried to collect solid in powder form. The powder is rehydrated to form a stable emulsion.

In another embodiment, a rice flour emulsifier is used to emulsify a lycopene oleoresin and an emulsion is formed. The emulsion is spray-dried to collect solid in powder form. The powder is rehydrated to form a stable emulsion.

In another embodiment, a rice flour emulsifier is used to emulsify a vegetable oil that contained paprika oleoresin and lycopene oleoresin and an emulsion is formed. The emulsion is oven-dried to collect solid in powder form. The powder is rehydrated to form a stable emulsion.

In another embodiment, a rice flour emulsifier and a corn flour emulsifier are combined to emulsify a vegetable oil that contains paprika oleoresin and lycopene oleoresin and an emulsion is formed. The emulsion is oven-dried to collect solid in powder form. The powder is rehydrated to form a stable emulsion.

In another embodiment, a rice flour emulsifier and a corn flour emulsifier are combined to emulsify a vegetable oil that contains paprika oleoresin and lycopene oleoresin and an emulsion is formed. The emulsion is drum-dried to collect solid in powder form. The powder is rehydrated to form a stable emulsion.

In another embodiment, a corn flour emulsifier is used to emulsify astaxanthin oleoresin and an emulsion is formed. The emulsion is freeze-dried to collect solid in powder form. The powder is rehydrated to form a stable emulsion.

In another embodiment, a rice flour emulsifier is used to emulsify capsicum oleoresin and an emulsion is formed. The emulsion is freeze-dried to collect solid in powder form. The powder is rehydrated to form a stable emulsion.

In another embodiment, a rice flour emulsifier is used to emulsify a vegetable that contained curcumin. Curcumin solid is first dispersed in the vegetable oil, and thereafter an emulsion is formed using the rice flour emulsifier. The emulsion is spray-dried to collect solid in powder form. The powder is rehydrated to form a stable emulsion.

In another embodiment, a barley flour emulsifier is used to emulsify beta-carotene. Beta-carotene solid is first dispersed in vegetable oil, and thereafter an emulsion is formed using the barley flour emulsifier. The emulsion is spray-dried to collect solid in powder form. The powder is rehydrated to form a stable emulsion.

In another embodiment, a pea flour emulsifier is used to emulsify lutein.

Lutein solid is first dispersed in vegetable oil, and thereafter an emulsion is formed using the pea flour emulsifier. The emulsion is spray-dried to collect solid in powder form. The powder is rehydrated to form a stable emulsion.

In another embodiment, a rice flour emulsifier is used to emulsify co-enzyme Q 10. Co-enzyme Q 10 is first diluted in a vegetable oil, and thereafter an emulsion is formed using the rice flour emulsifier. The emulsion is spray-dried to collect solid in powder form. The powder is rehydrated to form a stable emulsion.

Traditionally, flours without chemical treatment have been used as a food component. Traditional flours without chemical treatment does not process emulsification properties described herein, and have not been used as an emulsifier in food, beverage, personal care, agriculture, or industrial products.

This invention is about a flour emulsifier described herein.

This invention is about a process described herein making a flour emulsifier.

This invention is about a flour emulsifier made by the process disclosed.

This invention is about the uses of a flour emulsifier described herein.

This invention is about a product, wherein the product is a food, beverage, drug, medical product, personal care product, cosmetics, agriculture product, or industrial product, containing a flour emulsifier herein.

In another embodiment, an emulsifier described in this invention is used to make a bakery food, including but not limited to cake, muffin, donut, cookie, bread, flat bread, pie, cracker, chip, tortilla, pudding, bar, dessert, icing, topping, filling, candy, frozen dessert, refrigerated or frozen dough, snack food, and the like.

This invention is related to a new bakery food that contains a flour emulsifier described, said bakery food include but not limited to cake, muffin, donut, cookie, bread, flat bread, pie, cracker, chip, tortilla, pudding, bar, dessert, icing, topping, filling, candy, frozen dessert, refrigerated or frozen dough, snack food, and the like.

In another embodiment, an emulsifier described in this invention is used to make a food ingredient or food component, including but not limited to oleoresin, encapsulation, color formulation, flavor formulation, natural extract formulation, antioxidants formulation, nutrient formulation, DHA formulation, vitamin formulation, micro nutrients additives, dietary supplement, supplement ingredient, bakery ingredients mix, beverage ingredients mix, and the like.

This invention is related to a new food ingredient or food component that contains a flour emulsifier described, said food ingredient or food component include but not limited to oleoresin, encapsulation, color formulation, flavor formulation, natural extract formulation, antioxidants formulation, nutrient formulation, DHA formulation, vitamin formulation, micro nutrients additives, dietary supplement, supplement ingredient, bakery ingredients mix, beverage ingredients mix, and the like.

In another embodiment, an emulsifier described in this invention is used to make a frozen food, including frozen entries, dairy or non-dairy ice cream, frozen yoghurt, frozen dessert, frozen dough, frozen food component, frozen meal, frozen sauce, and the like.

This invention is related to a new frozen food that contains a flour emulsifier described, said frozen food include but not limited to frozen entries, dairy or non-dairy ice cream, frozen dough, frozen food component, frozen meal, frozen sauce, frozen yoghurt, frozen dessert, and the like.

In another embodiment, an emulsifier described in this invention is used to make a condiment or culinary food, including but not limited to sauce, dressing, soup, entries, sprinkle, food decoration, fillings, inclusions, and the like.

This invention is related to a new condiment or culinary food that contains a flour emulsifier described, said condiment or culinary food include but not limited to sauce, dressing, soup, entries, sprinkle, food decoration, fillings, inclusions, and the like.

In another embodiment, an emulsifier described in this invention is used to make a beverage, including but not limited to protein drink, energy drink, meal replacer, dried beverage powder, dairy and non-dairy beverage, almond milk, nut milk, soy milk, yoghurt, probiotic or prebiotic drink, nutrition supplement, shake, smoothie, and the like.

This invention is related to a beverage that contains a flour emulsifier described, said beverage include but not limited to protein drink, energy drink, meal replacer, dried beverage powder, dairy and non-dairy beverage, almond milk, nut milk, soy milk, yoghurt, probiotic or prebiotic drink, nutrition supplement, shake, smoothie, and the like.

In another embodiment, an emulsifier described in this invention is used to make a meat product, including but not limited to sausage, deli meat, canned meat, meat brine, alternative plant sourced meat, plant protein product, fungi protein product, plant burger, plant chicken, plant fish, and the like.

This invention is related to a meat or alternative protein product that contains a flour emulsifier described, said meat or alternative protein product include but not limited to sausage, deli meat, canned meat, meat brine, alternative plant sourced meat, plant protein product, fungi protein product, plant sourced burger, plant sourced chicken, plant sourced fish, and the like.

In another embodiment, an emulsifier described in this invention is used to make a feed product, including animal feed, feed additives, pet food, fish feed, and the like.

This invention is related to a feed protein product that contains a flour emulsifier described, said feed include but not limited to animal feed, feed additives, pet food, fish feed, feed ingredients, and the like.

In another embodiment, an emulsifier described in this invention is used to make a personal care product, including cream, lotion, moisturizer, skincare product, cosmetics, powder, foundation, eye shadow, bronzer, makeup, cleanser, serum, sunscreen, shampoo, conditioner, soap, hair product, detergent, dishwasher, wipe, baby powder, ointment, balm, lip product, household spray, fabric spray, fabric coating, and the like.

This invention is related to a personal care product that contains a flour emulsifier described, said personal care product include but not limited to cream, lotion, moisturizer, skincare product, cosmetics, powder, foundation, eye shadow, bronzer, makeup, cleanser, serum, sunscreen, shampoo, conditioner, soap, hair product, detergent, dishwasher, wipe, baby powder, ointment, balm, lip product, household spray, fabric spray, fabric coating, and the like.

In another embodiment, an emulsifier described in this invention is used to make a pharmaceutical product, including drug, antibiotics, anti-infection drug, anti-viral drug, anti-fungal drug, anti-cancer drug, vaccine, steroid, nasal spray, topical cream, ointment, injection, spray, medical drink, medical food, and the like.

This invention is related to a pharmaceutical product that contains a flour emulsifier described, said pharmaceutical product include drug, antibiotics, anti-infection drug, anti-viral drug, anti-fungal drug, anti-cancer drug, vaccine, steroid, nasal spray, topical cream, ointment, injection, spray, medical drink, medical food, and the like.

In another embodiment, an emulsifier described in this invention is used to make an agriculture product, including pesticide, herbicide, biocide, plant protection, plant nutrients, fertilizer, spray, plant hormone, seed protection, biocides, seed coating, fungicides, organic plant protection or nutrients, and the like.

This invention is related to an agriculture product that contains a flour emulsifier described, said agriculture product include but not limited to pesticide, herbicide, biocide, plant protection, plant nutrients, fertilizer, spray, plant hormone, seed protection, biocides, seed coating, fungicides, organic plant protection or nutrients, and the like.

In another embodiment, an emulsifier described in this invention is used to make an industrial product, including plastic, rubber, container, utensil, packaging, tire, cloth, fabric, film, leather, industrial spray, industrial cleaning liquid, and the ingredients of any above products.

This invention is related to an agriculture product that contains a flour emulsifier described, said industrial product include but not limited to plastic, rubber, container, utensil, packaging, tire, cloth, fabric, film, leather, industrial spray, industrial cleaning liquid, and the ingredients making above products.

In another embodiment, this invention is related to products described herein, wherein containing a flour emulsifier, and other ingredients.

This invention is related to combined use of flour emulsifier described together with other ingredients in an emulsion, ingredient, or product, said other ingredients include but not limited to other emulsifiers, stabilizers, bulking agents, rheological property modifiers, proteins, prebiotics, probiotics, and any other food, personal care, drug, agricultural, or industrial ingredients.

This invention is related to combined use of flour emulsifier described together with other ingredients in an emulsion, ingredient, or product, said other ingredients include but not limited to small molecule emulsifiers, mono-di glycerides, polysorbates, calcium stearoyl di laciate, sodium stearoyl lactylate, polyglycerol ester, sorbitan ester, propylene glycol ester, sugar ester, acetylated monoglyceride, lactylated monoglycerides, lecithin, modified starch, Gum Arabic, protein-based emulsifiers such as pea flour, sodium caseinate, whey protein isolates, starch, maltodextrins, syrups, sugars, sugar alcohols, oligosaccharides, hydrolyzed biopolymers, polysaccharides hydrolysates, protein hydrolysates, polysaccharide gums, xanthan gum, locus bean gum, guar gum, cellulose, carboxymethylcellulose, modified cellulose, starch, protein-based hydrocolloids, gelatin, soy protein, pea protein, egg white, pea proteins, soy proteins, cricket powder, protein hydrolysates, egg whites, pea flour, bean flours, lentil flour, prebiotics, probiotics, microbiome related materials, and the like.

In this invention, the purpose of milling is not only to break solid materials into smaller pieces, but also ensure a sufficient mixing of components in the flour, such as starch and protein. There are a variety of mills that can be used for dry materials, including but not limited to hammer mill, ball mill, rod mill, grinding roll, stone mill, jet mill, mortar and pestle, disc mill etc. For wet and semi-wet (semi-dry) materials, the milling or shearing can be conducted using (but not limited to) extruder, blender, sigma blender, colloid mill, high-speed homogenizer, high-pressure homogenizer, and fluidizer etc.

The heating can be conducted in a variety of ways, including but not limited to oven heating with or without ventilation, oven heating with or without vacuum, heating in a stirred vessel using direct hot air or through a jacket, and microwave. The purpose of heating is to control the temperature of materials at required range. The temperature can be maintained at a constant level or can change.

The protein content of flour material should be sufficient for bringing emulsification properties. In general, the protein content of flour materials is >1% and can be as high as 85%. For most flour materials, the protein content usually ranges 2%-50%.

The starch content of flour material should be sufficient to stabilize emulsions. In general, the starch content of flour material is over 20% and can be as high as over 90%. For most flour materials, the starch content usually ranges 15%-85%.

The crystallinity of starch indicates the percentage of crystalline regions in starch materials. The most common approach of determining starch crystallinity is to use X-ray powder diffraction. Usually the crystallinity of starch is around 20%-80%, depending on the types of starch. To have a desirable dispersing property, emulsification property, or a combination of both properties, the crystallinity of starch is reduced. The crystallinity of starch can be reduced to 90% or less of the original level, which means that the crystallinity of starch in the processed plant material equals to or is less than 90% of the crystallinity of starch in the original plant material. For example, if the starch in the original plant material has a crystallinity of 40% and after processing the starch crystallinity is 36%, then the crystallinity of starch is 90% of the crystallinity of starch in the original plant material. For another example, if the starch in the original plant material has a crystallinity of 40% and after processing the starch crystallinity is 30%, then the crystallinity of starch is 75% of the crystallinity of starch in the original plant material. In this invention, the crystallinity of starch in the processed material can be 0%-90% of the crystallinity of starch in the original plant material.

A heating only processing method of original plant materials (e.g. cereal and legume seeds, tubers, roots), or a milling only processing method may not lead to the same results as combined heating and milling process method. However, the original plant material may be optionally subject to hydrothermal treatments such as gelatinization, boiling, or steaming before subjecting to the combined heating and milling process described.

In one embodiment, a rice flour is subjected to a pre-gelatinization process and thereafter subjected to a heating and milling process that includes 2 hours of heating at 110° C. and 40 minutes of milling at a power input of 0.5 kw per kg of rice flour, thus to generate a flour emulsifier.

In another embodiment, a corn flour is subjected to an annealing process and thereafter subjected to a heating and milling process that includes 2 hours of heating at 110° C. and 90 minutes of milling at a power input of 0.3 kw per kg of corn flour, thus to generate a flour emulsifier.

In another embodiment, a rice flour is subjected to a pre-gelatinization process and a corn flour is subjected to an annealing process, and thereafter the rice flour and corn flour are combined and subjected to a heating and milling process that includes 2 hours of heating at 120° C. and 60 minutes of milling at a power input of 0.8 kw per kg of flour, thus to generate a flour emulsifier.

In another embodiment, a barley flour is subjected to a cooking process and then subjected to a heating and milling process that includes 1.5 hours of heating at 110° C. and 120 minutes of milling at a power input of 1 kw per kg of flour, thus to generate a flour emulsifier.

In another embodiment, a wheat flour is subjected to an autoclaving process and then subjected to a heating and milling process that includes 2.5 hours of heating at 110° C. and 120 minutes of milling at a power input of 0.7 kw per kg of flour, thus to generate a flour emulsifier.

In another embodiment, a pea flour is subjected to a steaming process and then subjected to a heating and milling process that includes 2 hours of heating at 110° C. and 120 minutes of milling at a power input of 0.3 kw per kg of flour, thus to generate a flour emulsifier.

This invention is also related to an emulsifier composition that comprises protein component (1%-85%) and carbohydrate component (15%-99%) including starch, wherein the protein component and carbohydrate component including starch are not necessarily from the same original material, wherein the crystallinity of starch in the emulsifier composition is less than 90% of the crystallinity of starch in the original plant material. The protein component and the carbohydrate component including starch can be obtained or isolated from one or a plurality of plant, animal, and/or microbial resources.

This invention is also related to an emulsifier composition that comprises protein component (1%-85%) and carbohydrate component (15%-99%), wherein the protein component and carbohydrate component are not necessarily from the same original material, and wherein a mixture of protein component and carbohydrate component is subjected to heating and milling to generate the said emulsifier composition. The protein component and the carbohydrate component can be obtained or isolated from one or a plurality of plant, animal, and/or microbial resources.

This invention is also related to an emulsifier composition that comprises protein component (1%-85%) and carbohydrate component (15%-99%), wherein the protein component and carbohydrate component are not necessarily from the same original material, wherein a mixture of protein component and carbohydrate component is subjected to heating and milling to yield the said emulsifier composition, wherein the heating process is conducted at a temperature from about 40° C. to about 300° C. for about 2 min to about 100 hours, and wherein the milling process is conducted for about 2 min to about 50 hours with a power input or power consumption of not less than 0.05 kw per kg of material processed. The protein component and the carbohydrate component can be obtained or isolated from one or a plurality of plant, animal, and/or microbial resources.

This invention is also related to the use of a milling process to generate emulsifier, wherein the milling process provides a power input, power output, and/or power consumption of not less than 0.05 kw (kilowatts), 0.1 kw, 0.2 kw, 0.3 kw, 0.4 kw, 0.5 kw, 0.6 kw, 0.7 kw, 0.8 kw per kg (kilogram) of processed material.

A number of specific examples or embodiments are provided herein to illustrate the present invention without limiting the scope of current invention.

In some examples, the preparations of various flour emulsifiers are described and their capabilities to stabilize emulsions are indicated using images.

The starch content and the starch crystallinity before and after the combined milling and heating treatment were determined for the flour.

The starch content was determined using Megazyme's total starch assay procedure. The Megazyme's kit include:
Thermostable alpha-amylase, 3000 U/mL, stabilized solution; 1 mL of enzyme solution was diluted with 29 mL sodium acetate buffer (100 mM, pH5.0)
Amyloglucosidase, 330 U/mL, stabilized solution;
Glucose determination reagent (GOPOD), glucose oxidase >2000 U/L, peroxidase >650 U/L, 4-aminoantipyrine 0.4 mM;
Glucose reagent buffer;
Glucose standard solution, 100 μg/0.1 mL in 0.2% benzoic acid;
Ethanol, aqueous, 80% v/v.
   To determine the total starch content of each individual sample, 100 mg of material was added in 15 mL centrifuge tube. The sample was moistened with 0.2 mL of 80% (v/v) ethanol and stirred using a vortex mixer to aid in dispersion. To each dispersed sample, 3 mL of the diluted thermostable alpha-amylase was added, and the mixture was incubated in a boiling-water bath for 12 minutes. To ensure the homogeneity of the slurry, the test tube was agitated at various time intervals (4, 8 and 12 minutes).
   After heating, the sample was allowed to cool and equilibrate at 50° C. in a water bath. To each tube, 0.1 mL of amyloglucosidase was added, and the mixture was incubated at 50° C. for 30 minutes. Thereafter, the content of the tube was transferred to a 100 mL volumetric flask, and the volume was adjusted using distilled water. From the volumetric flask, 1.00 mL of the dispersion was aspirated and centrifuged at 845×g for 10 min. From the supernatant, an aliquot (50 μL) was dispensed in a glass test tube and then GOPOD reagent (1.5 mL) was added, followed with 20 min of incubation at 50° C. After incubation, measure the absorbance for each sample at 510 nm against the reagent blank.
   The total starch content was calculated as following:

Starch (%)=ΔA×F×(FV/0.05)×(1/1000)×(100/W)×(162/180)

### Where:

ΔA=absorbance read against the reagent blank; [symbol]
F=100/(absorbance for 100 μg of glucose)
FV: final volume (100 mL)
0.05: volume of sample analyzed
1/1000: conversion factor from μg to mg
100/W: factor to indicate “starch” as a percentage of flour by weight
W: the weight in mg of the flour
162/180=adjustment from free D-glucose to anhydro D-glucose

Starch crystallinity (%) was obtained using X-ray powder diffraction of the samples and software integration of crystalline areas of the resulting crystallograms. Samples (each 500 mg) were mounted in aluminum holders and then analyzed using a Philips PW3710 diffractometer equipped with Ni-filtered CuKα (1.5418 Å) radiation, with the tube operated at 40 kV and 25 mA. Wide-angle X-ray crystallograms were obtained at room temperature within the 10-35° 2θ range and scan rate of 0.5°/min. The crystallograms were smoothed for further analysis by Automated Powder Diffraction (APD) software (version 3.6). The raw data was then loaded for calculation of crystallinity (%) using Origin Pro 2018 software, version 95E. Crystallinity (%) was determined using total area and baseline area for each crystallogram in the 10-30° 2θ range. The baseline pattern was obtained by using points in the non-peak area. Crystallinity (%) was calculated as follows:

$$\% crystallinity = ( \frac{total area - background area}{total area} ) \times 100$$

Protein content was determined using nitrogen content measured by combustion method with a LECO model FP-2000 Nitrogen Analyzer (LECO Co., St. Joseph, Mich.). For each material, 0.2 g were placed in ceramic containers and then nitrogen was determined by combustion with its value multiplied by 5.75 to obtain the protein content. Combustion was performed at 1100° C. with a lance and purge flow of 1.8 and 4.2 L/min, 3 purge cycles, and cooler set at 5° C. EDTA was used as standard (9.56±0.02% N content as determined by manufacturer).

### Example 1: Rice Variety #1

Milled rice grains variety #1 were ground to pass an 80-mesh sieve. The flour (untreated rice flour #1, URF-1) obtained was treated with 2.5 h (hours) of heating at 110° C. and 4 h (hours) of ball-milling. The flour emulsifier (RFE) collected was coded as rice flour emulsifier #1 (RFE-1).

For the rice flour, the starch content was 72.8%, and the protein content was 7.35%. The starch crystallinity was 46.9% and 41% before and after the combined milling and heating treatment, respectively, indicating that the treatment reduced the starch crystallinity to about 87.4% (41/46.9=0.874) of its original value.

2.5 grams of RFE-1 was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the RFE-1 based emulsion, 3 additional emulsions were prepared as controls, including: 1) with no emulsifier, 2) with untreated rice flour #1 (URF-1) as emulsifier, and 3) with gum arabic as emulsifier. The emulsions were placed at room temperature (20° C.). Before photographing, all emulsions (aliquot of each sealed in a 50-mL tube) were shaken together for 10 s. After shaking, the tubes were photographed at various time intervals to record the physical stability of emulsions.

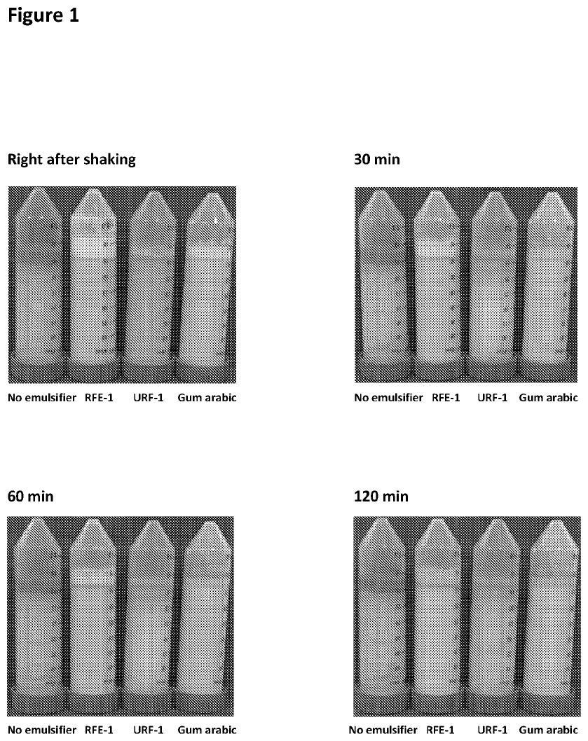

FIG. 1 shows the images of homogenized mixtures taken right after shaking (within 2 minutes after shaking) and at 30, 60, and 120 min after the shaking. The image taken at 120 min after the shaking showed that without the use of emulsifier (no emulsifier), the majority of oil droplets moved to the top of aqueous phase. For URF-1 stabilized emulsion, both creaming and sedimentation occurred, indicating the low stability of emulsion and thus the lack of emulsification capability of URF-1. For gum arabic stabilized emulsion, a layer of creaming occurred with its thickness much lower than that of no emulsifier and of URF-1. For RFE-1 stabilized emulsion, the creaming layer was comparable as that of gum arabic, showing a much greater emulsification capability of RFE-1 than URF-1.

The capability of a flour emulsifier to stabilize an oil-in-water emulsion can be evaluated and determined through comparing the images of homogenized mixture at various time intervals. For example, in FIG. 1, through comparing the images of “120 min” and “60 min”, it was found that the RFE-1 mixture at 120 min after shaking was more stable than the URF-1 mixture at 30, 60, and 120 min. Since 120 min is 4 times that of 30 min, we can conclude that the capability of RFE-1 to stabilize emulsion was at least 4 times that of URF-1.

As one method, this invention evaluates the capability of a flour emulsifier to stabilize an emulsion through the following procedure:
1) Observe, document, and compare the emulsion stability of the homogenized mixtures at various time intervals after shaking. For example, photographs of homogenized mixture can be taken at several time intervals.
2) Select a comparison mixture, which can be the homogenized mixture with no emulsifier, with a flour without combined treatment of milling and heating, or with a commercial emulsifier such as gum arabic. For example, the homogenized mixture with a flour without combined treatment of milling and heating can be used as a comparison mixture.
3) For the comparison mixture, select a time interval after the shaking, such as right after shaking (within 2 minutes after shaking) or at 30 min, 60 min, or 120 min after shaking. The selected comparison mixture and the selected time interval together form the comparison standard. For example, the homogenized mixture with untreated flour at 30 min after shaking can be used as the comparison standard.
4) Against the established comparison standard, the emulsification capability of flour emulsifier can be quantitatively evaluated. In this invention, the homogenized mixture with the flour emulsifier (at a certain time interval) is compared with the comparison standard. If the homogenized mixture with the flour emulsifier appears more stable (e.g. more evenly dispersed) than the comparison standard, then the ratio (N) between the time interval (after shaking) of the homogenized mixture with the flour emulsifier and the time interval used for the comparison standard is used to define that the flour emulsifier has the emulsification capability of “at least N times” that of the material used in the comparison standard.
5) For example, FIG. 1 shows that the homogenized mixture with RTE-1 after 120 min (i.e. at 120 min after the shaking) was more stable (or more evenly dispersed) than the homogenized mixture with URF-1 after 30 min (i.e. at 30 min after the shaking). Since 120 min is 4 times the duration of 30 min, we determine that RTE-1 has the emulsification capability at least 4 times that of URF-1. We also say that the stability of emulsion formed by RTE-1 was at least 4 times that formed by URF-1.

### Example 2: Rice Variety #2

Mill rice grains (variety #2) were ground to pass an 80-mesh sieve. The untreated rice flour #2 (URF-2) obtained was treated with a combination of 2.5 h of heating at 110° C. and 4 h of ball-milling. The flour emulsifier collected was coded as rice flour emulsifier #2 (RFE-2).

For the rice flour, the starch content was 73.0%, and the protein content was 6.65%. The starch crystallinity was 48.2% and 35.4% before and after the combined milling and heating treatment, respectively, indicating that the treatment reduced the starch crystallinity to about 73.4% of its original value.

2.5 grams of RFE-2 was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the RFE-2 based emulsion, 3 emulsions were prepared as controls, including: 1) with no emulsifier, 2) with untreated rice flour #2 (URF-2) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

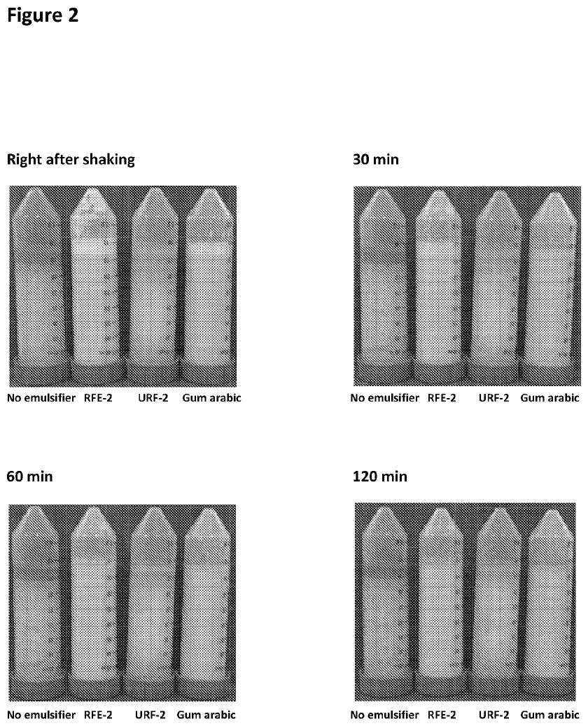

FIG. 2 shows the image taken right after shaking (within 2 minutes after shaking) and at 30, 60, and 120 min after shaking. For the “120 min” group, it is shown that without the use of emulsifier, the majority of oil droplets moved to the top of aqueous phase. For URF-2 stabilized emulsion, both creaming and sedimentation occurred, indicating the low stability of emulsion and thus the lack of emulsification capability of URF-2. For gum arabic stabilized emulsion, a layer of creaming occurred with its thickness much lower than that of no emulsifier and of URF-2. For RFE-2 stabilized emulsion, the creaming layer was much thinner than that of gum arabic, showing a greater emulsification capability of RFE-2 than URF-2 and gum arabic.

Since the emulsion with RFE-2 at 120 min after shaking was more stable (or more evenly dispersed) than the emulsion with URF-2 at 30 min, it was concluded that the emulsification capability of RFE-2 was at least 4 times (120/30=4) that of URF-2

Since the emulsion with RFE-2 at 120 min after shaking was more stable (or more evenly dispersed) than the emulsion with URF-2 right after shaking (within 2 min after shaking), it was concluded that the emulsification capability of RFE-2 was at least 60 times (120/2=60) that of URF-2.

Since the emulsion with RFE-2 at 120 min after shaking was more stable (or more evenly dispersed) than the emulsion with gum arabic at 60 min after shaking, it was concluded that the emulsification capability of RFE-2 was at least twice (120/60=2) that of gum arabic.

### Example 3: Rice Variety #2

Mill rice grains variety #2 were ground to pass an 80-mesh sieve. The untreated rice flour #2 (URF-2) obtained was subjected to 6 cycles of the combination of 30 min ball milling and 30 min heating at 110° C. The flour emulsifier collected was coded as RFE-2-1 (rice flour emulsifier #2-1).

For the rice flour, the starch content was 73.0%, and the protein content was 6.65%. The starch crystallinity was 48.2% and 36.4% before and after the combined milling and heating treatment, respectively, indicating that the treatment reduced the starch crystallinity to about 75.5% of its original value.

2.5 grams of RFE-2-1 was dispersed in 72.5 g distilled water. To dispersion, 5 grams of soybean oil (containing with 1% w/w paprika oleoresin to show color) was added. The mixture was homogenized for 10 times, each time for 15 second using a food processor. Along with the RFE-2-1 based emulsion, 3 emulsions were prepared as controls, including: 1) with no emulsifier, 2) with untreated rice flour #2 (URF-2) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

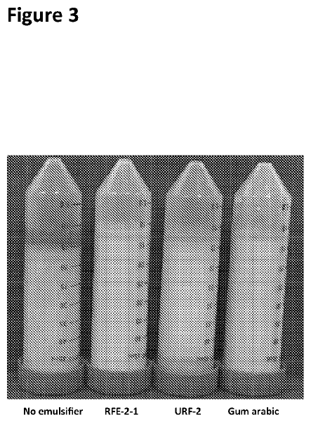

FIG. 3 shows the image taken at 120 min after tube shaking. Without the use of emulsifier, the majority of oil droplets moved to the top of aqueous phase. For URF-2 stabilized emulsion, both creaming and sedimentation occurred, indicating the low stability of emulsion and thus the lack of emulsification capability of URF-2. For gum arabic stabilized emulsion, a layer of creaming occurred with its thickness much lower than that of no emulsifier and of URF-2. For RFE-2-1 stabilized emulsion, the creaming layer was lighter and thinner than that of gum arabic, showing a greater emulsification capability of RFE-2-1 than URF-2 and gum arabic.

### Example 4: Rice Variety #3

Mill rice grains (variety #3) were ground to pass an 80-mesh sieve. The untreated rice flour #3 (URF-3) obtained was subjected to 10 cycles of the combination of 30 min milling and 30 min heating at 110° C. The flour emulsifier collected was coded as rice flour emulsifier #3 (RFE-3).

For the rice flour, the starch content was 79.1%, and the protein content was 5.5%. The starch crystallinity was 46.6% and 15% before and after the combined milling and heating treatment, respectively, indicating that the treatment reduced the starch crystallinity to about 32.2% of its original value.

2.5 grams of RFE-3 was dispersed in 72.5 g distilled water. To dispersion, 5 grams of soybean oil (containing with 1% w/w paprika oleoresin to show color) was added. The mixture was homogenized for 10 times, each time for 15 second using a food processor. Along with the RFE-3 based emulsion, 3 emulsions were prepared as controls, including: 1) with no emulsifier, 2) with untreated rice flour #3 (URF-3) as emulsifier, and 3) with gum arabic. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

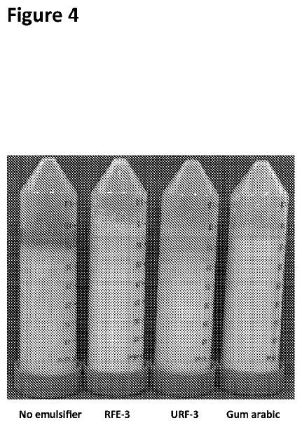

FIG. 4 shows the image taken at 120 min after tube shaking. Without the use of emulsifier, the majority of oil droplets moved to the top of aqueous phase. For URF-3 stabilized emulsion, both creaming and sedimentation occurred, indicating the low stability of emulsion and thus the lack of emulsification capability of URF-3. For gum arabic stabilized emulsion, a layer of creaming occurred with its thickness much lower than that of no emulsifier and of URF-3. For RFE-3 stabilized emulsion, the creaming layer was much lighter and thinner than that of URF-3, showing a greater emulsification capability of RFE-3 than that of URF-3.

### Example 5: Barley

Barley grains were ground to pass an 80-mesh sieve. The untreated barley flour (UBF) obtained was treated with 1 h of heating at 110° C., 4 h of ball-milling (Planetary ball mill, PQ-N2, 580 rpm), and again 1.5 h of heating at 110° C. The barley flour emulsifier (BFE) was thus collected.

For the barley flour, the starch content was 65.4%, and the protein content was 6.96%. The starch crystallinity was 27.5% and 9.7% before and after the combined milling and heating treatment, respectively, indicating that the treatment reduced the starch crystallinity to about 35.3% of its original value.

2.5 grams of BFE was dispersed in 72.5 g distilled water. To dispersion, 5 grams of soybean oil (containing with 1% w/w paprika oleoresin to show color) was added. The mixture was homogenized for 10 times, each time for 15 second using a food processor. Along with the BFE based emulsion, 3 emulsions were prepared as controls, including: 1) with no emulsifier, 2) with untreated barley flour (UBF) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

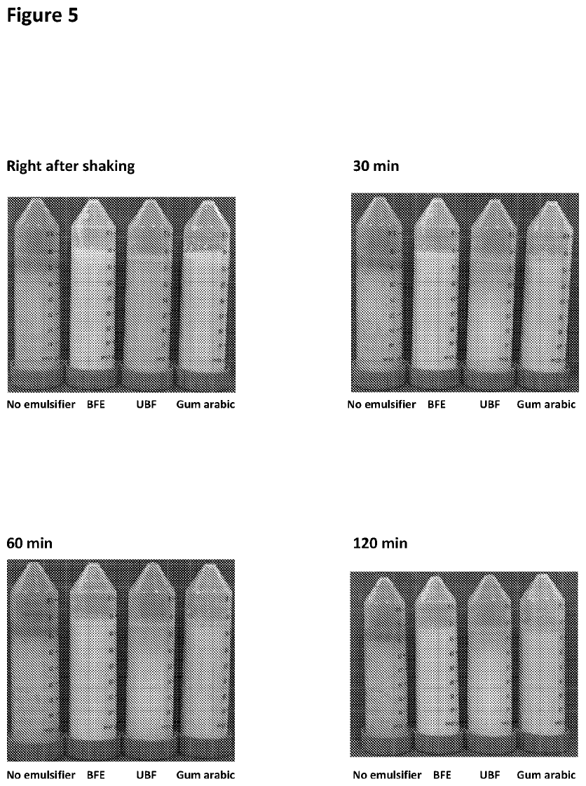

FIG. 5 shows the image taken right after shaking (within 2 min after shaking) and at 30, 60, and 120 min after shaking. For the “120 min” group, it is shown that without the use of emulsifier, the majority of oil droplets moved to the top of aqueous phase. For UBF stabilized emulsion, both creaming and sedimentation occurred, indicating the low stability of emulsion and thus the lack of emulsification capability of UBF. For gum arabic stabilized emulsion, a layer of creaming occurred with its thickness much lower than that of no emulsifier and of UBF. For BFE stabilized emulsion, the creaming layer was much lighter and thinner than that of UBF and gum arabic, showing a greater emulsification capability of BFE than that of UBF and gum arabic.

Since the emulsion with BFE at 120 min after shaking was more stable than the emulsion with UBF at 30 min after shaking, it was concluded that the emulsification capability of BFE was at least 4 times (120/30=4) that of UBF.

Since the emulsion with BFE at 120 min after shaking was more stable than the emulsion with UBF right after shaking (within 2 min after shaking), it was concluded that the emulsification capability of BFE was at least 60 times (120/2=60) that of UBF.

Since the emulsion with BFE at 120 min after shaking was more stable than the emulsion with gum arabic at 60 min after shaking, it was concluded that the emulsification capability of BFE was at least twice (120/60=2) that of gum arabic.

### Example 6: Rice Variety #4

Mill rice grains (variety #4) were ground to pass an 80-mesh sieve. The untreated rice flour #4 (URF-4) obtained was treated with a combination of 2.5 h of heating at 110° C. and 4 h of ball-milling. The flour emulsifier collected was coded as rice flour emulsifier #4 (RFE-4).

For the rice four, the starch content was 71.1%, and the protein content was 6.18%. The starch crystallinity was 45.5% and 25.9% before and after the combined milling and heating treatment, respectively, indicating that the treatment reduced the starch crystallinity to about 56.9% of its original value.

2.5 grams of RFE-4 was dispersed in 72.5 g distilled water. To dispersion, 5 grams of soybean oil (containing with 1% w/w paprika oleoresin to show color) was added. The mixture was homogenized 10 times, each time for 15 seconds using a food processor. Along with the RFE-4 based emulsion, 3 emulsions were prepared as controls, including: 1) with no emulsifier, 2) with untreated rice flour #4 (URF-4) as emulsifier, and 3) with gum arabic. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

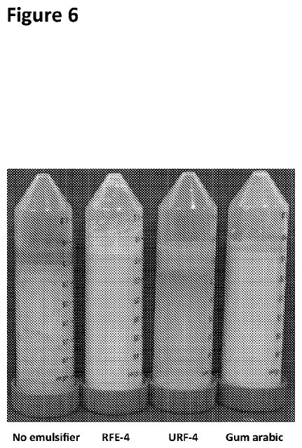

FIG. 6 shows the image taken at 120 min after tube shaking. Without the use of emulsifier, the majority of oil droplets moved to the top of aqueous phase. For URF-4 stabilized emulsion, both creaming and sedimentation occurred, indicating the low stability of emulsion and thus the lack of emulsification capability of URF-4. For gum arabic stabilized emulsion, a layer of creaming occurred with its thickness much lower than that of no emulsifier and of URF-4. For RFE-4 stabilized emulsion, the creaming layer was much lighter and thinner than that of URF-4 and gum arabic, showing a much greater emulsification capability of RFE-4 than that of gum arabic.

### Example 7: Wheat

Whole-wheat grains were ground to pass an 80-mesh sieve. The untreated wheat flour (UWF) obtained was treated with 2.5 h of heating at 110° C. and 4 h of ball-milling. The wheat flour emulsifier (WFE) was thus obtained.

For the wheat flour, the starch content was 59.9%, and the protein content was 10.31%. The starch crystallinity was 30.7% and 12.4% before and after the combined milling and heating treatment, respectively, indicating that the treatment reduced the starch crystallinity to about 40.4% of its original value.

2.5 grams of WFE was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the WFE based emulsion, 3 emulsions were prepared as controls, including: 1) with no emulsifier, 2) with untreated whole-wheat flour (UWF) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

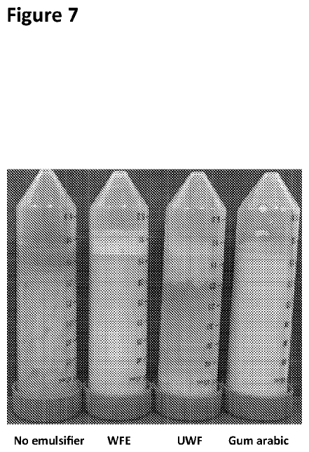

FIG. 7 shows the image taken at 120 min after tube shaking. Without the use of emulsifier, the majority of oil droplets moved to the top of aqueous phase. For UWF stabilized emulsion, both creaming and sedimentation occurred, indicating the low stability of emulsion and thus the lack of emulsification capability of UWF. For gum arabic stabilized emulsion, a layer of creaming occurred with its thickness much lower than that of no emulsifier and of UWF. For WFE stabilized emulsion, the creaming layer was much lighter and thinner than that of UWF, however, an overall separation started to occur, indicating its lower emulsification capability than that of gum arabic.

### Example 8: Degermed Corn Variety #1

Grits of degermed corn variety #1 were ground to pass an 80-mesh sieve. The flour (untreated corn flour #1, UCF-1) obtained was treated with 2.5 h of heating at 110° C. and 4 h of ball-milling. The corn flour emulsifier #1 (CFE-1) was thus collected.

For the corn flour, the starch content was 75.9%, and the protein content was 4.95%. The starch crystallinity was 41.7% and 12.1% before and after the combined milling and heating treatment, respectively, indicating that the treatment reduced the starch crystallinity to about 29% of its original value.

2.5 grams of CFE-1 was dispersed in 72.5 g distilled water. To dispersion, 5 grams of soybean oil (containing with 1% w/w paprika oleoresin to show color) was added. The mixture was homogenized for 10 times, each time for 15 seconds using a blender. Along with the CFE-1 based emulsion, 3 emulsions were prepared as controls, including: 1) with no emulsifier, 2) with untreated corn flour #1 (UCF-1) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

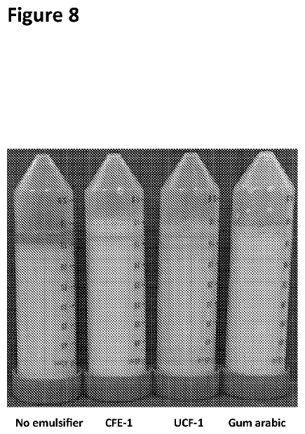

FIG. 8 shows the image taken at 120 min after tube shaking. Without the use of emulsifier, the majority of oil droplets moved to the top of aqueous phase. For UCF-1 stabilized emulsion, both creaming and sedimentation occurred, indicating the low stability of emulsion and thus the lack of emulsification capability of UCF-1. For gum arabic stabilized emulsion, a layer of creaming occurred with its thickness much lower than that of no emulsifier and of UCF-1. For CFE-1 stabilized emulsion, the creaming layer was much lighter and thinner than that of UCF-1 but similar to that of gum arabic, indicating a similar emulsification capability of CFE-1 to that of gum arabic.

### Example 9: Degermed Corn Variety #2

Grits of degermed corn variety #2 were ground to pass an 80-mesh sieve. The flour (untreated corn flour #2, UCF-2) obtained was treated with 2.5 h of heating at 110° C. and 4 h of ball-milling. The corn flour emulsifier #2 (CFE-2) was thus collected.

2.5 grams of CFE-2 was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the CFE-2 based emulsion, 3 emulsions were prepared as controls, including: 1) with no emulsifier, 2) with untreated corn flour (UCF-2) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

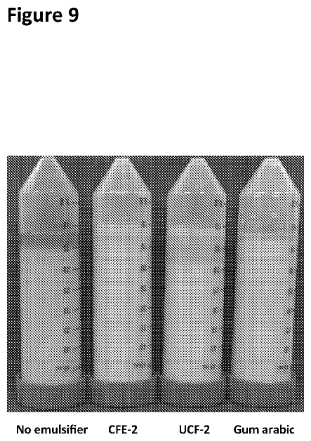

FIG. 9 shows the image taken at 120 min after tube shaking. Without the use of emulsifier, the majority of oil droplets moved to the top of aqueous phase. For UCF-2 stabilized emulsion, substantial creaming occurred, indicating the low stability of emulsion and thus the low emulsification capability of UCF-2. For gum arabic stabilized emulsion, a layer of creaming occurred with its thickness much lower than that of no emulsifier and of UCF-2. For CFE-2 stabilized emulsion, the creaming layer was much lighter and thinner than that of UCF-2 but similar to that of gum arabic, indicating a similar emulsification capability of CFE-2 to that of gum arabic.

### Example 10: Rice Variety #5

Mill rice grains variety #5 were ground to pass an 80-mesh sieve. The untreated rice flour #5 obtained was treated with 2.5 h of heating at 110° C. and 4 h of ball-milling. The rice flour emulsifier thus collected was coded as rice flour emulsifier #5 (RFE-5).

For the rice flour, the starch content was 71.9%, and the protein content was 6.08%. The starch crystallinity was 45.4% and 15.4% before and after the combined milling and heating treatment, respectively, indicating that the treatment reduced the starch crystallinity to about 33.9% of its original value.

2.5 grams of RFE-5 was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the RFE-5 based emulsion, 3 emulsions were prepared as controls, including: 1) with no emulsifier, 2) with untreated rice flour #5 (URF-5) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

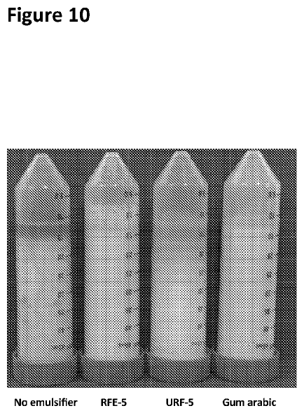

FIG. 10 shows the image taken at 120 min after tube shaking. Without the use of emulsifier, the majority of oil droplets moved to the top of aqueous phase. For URF-5 stabilized emulsion, substantial creaming occurred, indicating the low stability of emulsion and thus the lack of emulsification capability of URF-5. For gum arabic stabilized emulsion, a layer of creaming occurred with its thickness much lower than that of no emulsifier and of URF-5. For RFE-5 stabilized emulsion, the creaming layer was much thinner than that of URF-5 and similar to that of gum arabic, indicating a comparable emulsification capability of RFE-5 with gum arabic.

### Example 11: Rice Variety #6

Mill rice grains variety #6 were ground to pass an 80-mesh sieve. The untreated rice flour #6 (URF-6) obtained was treated with 2.5 h of heating at 110° C. and 4 h of ball-milling. The rice flour emulsifier collected was coded as RFE-6 (rice flour emulsifier #6).

For the rice flour, the starch content was 75.0%, and the protein content was 5.64%. The starch crystallinity was 48.6% and 19.6% before and after the combined milling and heating treatment, respectively, indicating that the treatment reduced the starch crystallinity to about 40.3% of its original value.

2.5 grams of RFE-6 was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the RFE-6 based emulsion, 3 emulsions were prepared as controls, including: 1) with no emulsifier, 2) with untreated rice flour #6 (URF-6) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

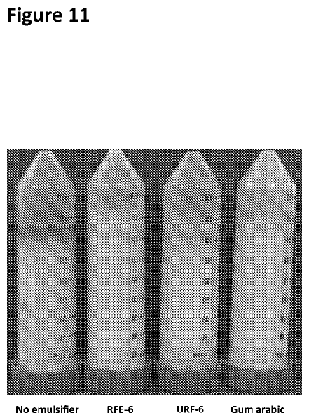

FIG. 11 shows the image taken at 120 min after tube shaking. Without the use of emulsifier, the majority of oil droplets moved to the top of aqueous phase. For URF-6 stabilized emulsion, substantial creaming and sedimentation occurred, indicating the low stability of emulsion and thus the lack of emulsification capability of URF-6. For gum arabic stabilized emulsion, a layer of creaming occurred with its thickness much lower than that of no emulsifier and of URF-6. For RFE-6 stabilized emulsion, the creaming layer was much thinner than that of URF-6 and similar to that of gum arabic, indicating a comparable emulsification capability of RFE-6 with gum arabic.

### Example 12: Northern Bean

Northern beans were soaked (4° C. overnight), steamed (60 min), dried (50° C. for 11 h), and then ground to pass an 80-mesh sieve to collect “untreated northern bean flour, UNBF”. UNBF was treated with 2.5 h of heating at 110° C. and 4 h of ball-milling. The flour obtained was northern bean flour emulsifier (NBFE).

For the northern bean flour, the starch content was 37.5%, and the protein content was 19.91%. The combined treatment of milling and heating reduced the starch crystallinity to about 70.1% of its original value.

2.5 grams of NBFE was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the NBFE based emulsion, 3 emulsions were prepared as controls, including: 1) with no emulsifier, 2) with untreated northern bean flour (UNBF) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

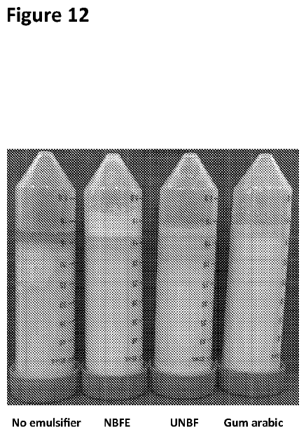

FIG. 12 shows the image taken at 120 min after tube shaking. Without the use of emulsifier, the majority of oil droplets moved to the top of aqueous phase. For UNBF stabilized emulsion, substantial creaming and sedimentation occurred, indicating the low stability of emulsion and thus the lack of emulsification capability of UNBF. For gum arabic stabilized emulsion, a layer of creaming occurred with its thickness much lower than that of no emulsifier and of UNBF. For NBFE stabilized emulsion, the creaming layer was much thinner than that of UNBF but thicker than that of gum arabic, indicating an increased emulsification capability of northern bean flour due to the combined heating and milling treatment.

### Example 13: Kidney Bean

Kidney beans were soaked (4° C. overnight), steamed (60 min), dried (50° C. for 11 h), and then ground to pass an 80-mesh sieve to collect “untreated kidney bean flour, UKBF”. UKBF was treated with 2.5 h of heating at 110° C. and 4 h of ball-milling. The flour obtained was kidney bean flour emulsifier (KBFE).

2.5 grams of KBFE was dispersed in 72.5 g distilled water. To dispersion, 5 grams of soybean oil (containing with 1% w/w paprika oleoresin to show color) was added. The mixture was homogenized for 10 times, each time for 15 seconds using a blender. Along with the KBFE based emulsion, 3 emulsions were prepared as controls, including: 1) with no emulsifier, 2) with untreated kidney bean flour (UKBF) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

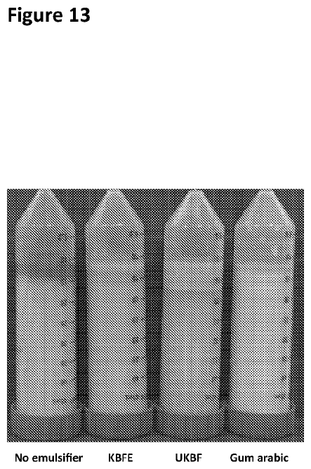

FIG. 13 shows the image taken at 120 min after tube shaking. Without the use of emulsifier, the majority of oil droplets moved to the top of aqueous phase. For UKBF stabilized emulsion, substantial creaming and sedimentation occurred, indicating the low stability of emulsion and thus the lack of emulsification capability of UKBF. For gum arabic stabilized emulsion, a layer of creaming occurred with its thickness much lower than that of no emulsifier and of UKBF. For KBFE stabilized emulsion, the creaming layer was lighter than that of UKBF but thicker than that of gum arabic, indicating an increased emulsification capability of kidney bean flour due to the combined heating and milling treatment.

### Example 14: Rice Variety #2

Rice grains (variety #2) were ground to pass an 80-mesh sieve and then the flour (untreated rice flour #2, URF-2) obtained was subjected to combined treatment of heating at 110° C. for 90 min and milling for 2 h. The product collected was termed as RFE-2-2.

For the rice flour, the starch content was 73.0%, and the protein content was 6.65%. The starch crystallinity was 48.2% and 9.3% before and after the combined milling and heating treatment, respectively, indicating that the treatment reduced the starch crystallinity to about 19.3% of its original value.

2.5 grams of RFE-2-2 was dispersed in 72.5 g distilled water. To dispersion, 5 grams of soybean oil (containing with 1% w/w paprika oleoresin to show color) was added. The mixture was homogenized for 10 times, each time for 15 seconds using a food processor. Along with the RFE-2-2 based emulsion, 3 emulsions were prepared as controls, including: 1) with no emulsifier, 2) with untreated rice flour #2 (URF-2) as emulsifier, and 3) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

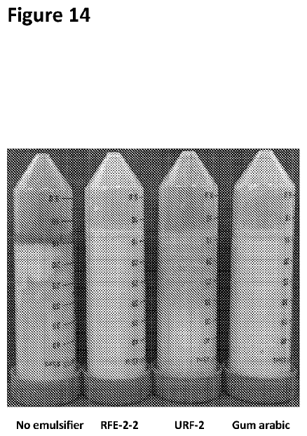

FIG. 14 shows the image taken at 120 min after tube shaking. Without the use of emulsifier, the majority of oil droplets moved to the top of aqueous phase. For URF-2 stabilized emulsion, both creaming and sedimentation occurred, indicating the low stability of emulsion and thus the lack of emulsification capability of URF-2. For gum arabic stabilized emulsion, a layer of creaming occurred with its thickness much lower than that of no emulsifier and of URF-2. For RFE-2-2 stabilized emulsion, the creaming layer was lighter and thinner than that of gum arabic, showing a greater emulsification capability of RFE-2-2 than URF-2 and gum arabic.

### Example 15: RFE-2-2 to Form Emulsion of Retinol-Soybean Oil Mixture

2.5 grams of RFE-2-2 was dispersed in 72.5 g distilled water. To dispersion, 5 grams of retinol-soybean oil mixture (10% w/w retinol in soybean oil) was added. The mixture was homogenized for 10 times, each time for 15 seconds using a food processor. Along with the RFE-2-2 based emulsion, 2 emulsions were prepared as controls, including: 1) with no emulsifier, and 2) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

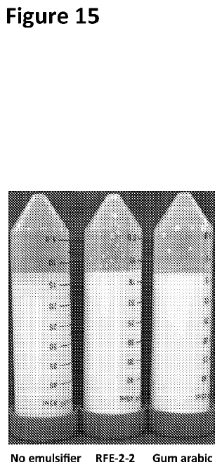

FIG. 15 shows the image taken at 120 min after tube shaking. Without the use of emulsifier, the majority of oil droplets moved to the top of aqueous phase. For gum arabic stabilized emulsion, a layer of creaming occurred with its thickness much lower than that of no emulsifier. For RFE-2-2 stabilized emulsion, the creaming layer was nearly invisible, showing a greater emulsification capability of RFE-2-2 than gum arabic for retinol-soybean oil mixture.

### Example 16: RFE-2-2 Enables Tocopherol Dispersing in Water

2.5 grams of RFE-2-2 was dispersed in 72.5 g distilled water. To dispersion, 5 grams of tocopherol was added. The mixture was homogenized for 10 times, each time for 15 seconds using a food processor. Along with the RFE-2-2 based emulsion, 2 emulsions were prepared as controls, including: 1) with no emulsifier, and 2) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

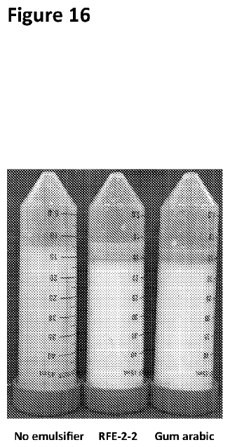

FIG. 16 shows the image taken at 120 min after tube shaking. Without the use of emulsifier, the majority of oil droplets separated from the water phase. For gum arabic stabilized emulsion, a layer of creaming occurred. For RFE-2-2 stabilized emulsion, the creaming layer also formed, however, with lower density, showing the emulsification capability of RFE-2-2 comparable with or superior to that of gum arabic for tocopherol.

### Example 17: RFE-2-2 to Form Emulsion of Astaxanthin

2.5 grams of RFE-2-2 was dispersed in 72.5 g distilled water. To dispersion, 5 grams of astaxanthin oleoresin (10%) was added. The mixture was homogenized for 10 times, each time for 15 seconds using a food processor. Along with the RFE-2-2 based emulsion, 2 emulsions were prepared as controls, including: 1) with no emulsifier, and 2) with gum arabic as emulsifier. The emulsions were placed at room temperature (20° C.). Before photographing, all emulsions (aliquot of each sealed in a 50-mL tube) were shaken for 10 s. After shaking, the tubes were photographed at various time intervals to record the physical stability of emulsions.

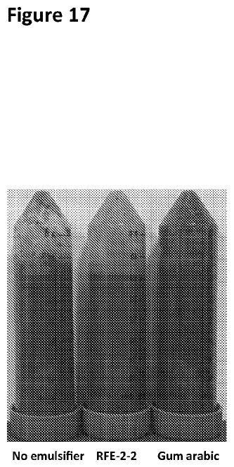

FIG. 17 shows the image taken at 120 min after tube shaking. Without the use of emulsifier, the astaxanthin oleoresin cannot be well dispersed and thus form paste dots along the wall of tube. For gum arabic, the oleoresin was better dispersed but a substantial amount of paste still attached at the wall surface. For RFE-2-2, the paste was nearly negligible due to a much better dispersion formed. Apparently, RFE-2-2 has a much greater capability to disperse and emulsify astaxanthin oleoresin than gum arabic.

### Example 18: RFE-2-2 Protected Paprika Oleoresin Emulsion from Light-Triggered Degradation

The mixture that contained paprika oleoresin, RFE-2-2, and water in a weight-based ratio of 1:3:10 was subjected to homogenization to prepare an emulsion. As the control, gum arabic was used to replace RFE-2-2 to prepare an emulsion with the same ratios among oil, emulsifier, and water as that for the RFE-2-2 group. Both emulsions were diluted with water for a concentration of paprika oleoresin around 200 μg/mL. The diluted emulsions were exposed to light treatment for 48 hours.

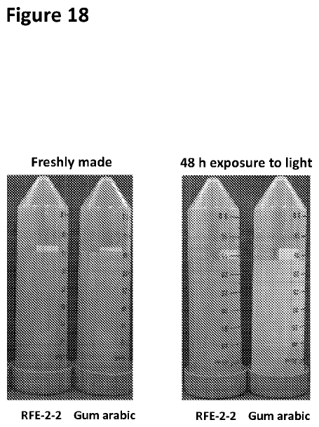

As shown in the FIG. 18, both REF-2-2 and gum arabic were able to form emulsions of paprika oleoresin. After 48 h exposure to light, the color of gum arabic based emulsion was substantially reduced, whereas the color of RFE-2-2 emulsion nearly retained its original strength. Therefore, the used of REF-2-2 as an emulsifier was able to protect the coloring component in paprika oleoresin from being degraded due to the light exposure.

### Example 19: Combined Heating and Milling of Untreated Flour is Needed to Yield Flour Emulsifier with Acceptable Emulsification Properties

Mill rice grains (variety #2) were ground to pass an 80-mesh sieve. One portion of the untreated rice flour #2 (URF-2) obtained was subjected to a combined treatment of 2.5 h heating at 110° C. and 4 h ball-milling. The flour emulsifier collected was coded as rice flour emulsifier #2 (RFE-2). The second portion of URF-2 was only treated with 4 h of ball milling (without heating), and the flour collected was coded as URF-2B.

2.5 grams of each of RFE-2 and URF-2B was dispersed in 72.5 g distilled water. To dispersion, 5 grams of soybean oil (containing with 1% w/w paprika oleoresin to show color) was added. The mixture was homogenized for 10 times, each time for 15 seconds using a food processor. The emulsions were placed at room temperature (20° C.). Before photographing, both emulsions (aliquot of each sealed in a 50-mL tube) were shaken for 10 s. After shaking, the tubes were photographed at various time intervals to record the physical stability of emulsions.

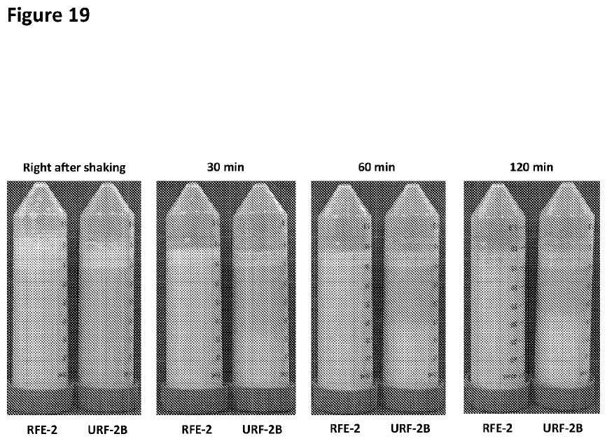

FIG. 19 shows the images of homogenized mixtures taken right after shaking (within 2 minutes after shaking) and at 30, 60, and 120 min after shaking. It is shown that at any point, the emulsion formed with RFE-2 was much more stable than the emulsion formed with URF-2B, demonstrating the role of combined heating and milling on producing flour emulsifier with superior emulsification properties, compared to milling-only treatment.

When regular flour (e.g. URF-2) was heated only, the material generated (URF-2H) did not show acceptable emulsification capability. In general, heating only (e.g. drying heating, steaming, microwaving, boiling) without applying any shear (e.g. milling or extrusion) does not generate flour emulsifier with acceptable emulsification capability.

Therefore, it is essential to apply a combined heating and milling to afford a flour emulsifier with acceptable and/or superior emulsification properties.

Other processes that apply shear force, such as extrusion, may also enhance the emulsification capability of a flour if the shear process is conducted in combination with a heating process.

### Example 20. Isolated Starch Subjected to Combined Heating and Milling Did not Show Emulsification Capability

Mill rice grains (variety #2) were ground to pass an 80-mesh sieve to produce the untreated rice flour #2 (URF-2).

Starch was isolated from the URF-2 using the following procedure.

To start the extraction, 100 g of milled rice grains were ground into grits using a food processor to pass through a 16-mesh sieve. The grits were mixed with 350 mL of 0.1% (w/v) sodium hydroxide (NaOH) solution and kept at 50° C. in a water bath for 30 min with constant agitation. The mixture was then homogenized using a food processor at high speed for 4 min and passed through a 270-mesh sieve. The retained solids (by the sieve) were extracted again using another 350 mL of NaOH solution. The fractions permeated through the sieve were combined and centrifuged at 3,000×g for 15 min. The precipitate, as the crude starch material, was collected.

Crude starch material was re-suspended in 300 mL of NaOH solution (pH 10), agitated for 30 min, and centrifuged again. The precipitate was washed with NaOH solution for four times, during which proteins on the top layer of the precipitate was scrapped away using a spatula. Thereafter, the precipitate was re-suspended in deionized water, neutralized to pH 7.0 using 1.0 M hydrogen chloride (HCl) solution, and centrifuged. The starch precipitate was further washed twice with deionized water and once with ethanol. The material collected was subjected to vacuum filtration and then dried overnight in a fume hood. The dried starch material was collected, and the protein content of this starch was 0.97%.

Both URF-2 and isolated rice starch were subjected to a combined heating and ball milling process that included heating at 110° C. for 2.5 h and ball milling for 4 h. The flour emulsifier collected from the combined heating and milling treatment of URF-2 was coded as rice flour emulsifier #2 (RFE-2); the starch material collected from the combined heating and milling treatment of isolated rice starch was coded as Starch-HB.

For the rice flour, the combined heating and ball milling treatment reduced the starch crystallinity to about 73.4% of its original value. For the isolated starch, the starch crystallinity before and after the combined heating and milling treatment was 57.8% and 9.91%, respectively, indicating that the treatment reduced the starch crystallinity to about 17.1% or its original value.

2.5 grams of each of RFE-2 and Starch-HB was dispersed in 72.5 g distilled water. To dispersion, 5 grams of soybean oil (containing with 1% w/w paprika oleoresin to show color) was added. The mixture was homogenized for 10 times, each time for 15 seconds using a food processor. The emulsions were placed at room temperature (20° C.). Before photographing, both emulsions (aliquot of each sealed in a 50-mL tube) were shaken for 10 s. After shaking, the tubes were photographed at various time intervals to record the physical stability of emulsions.

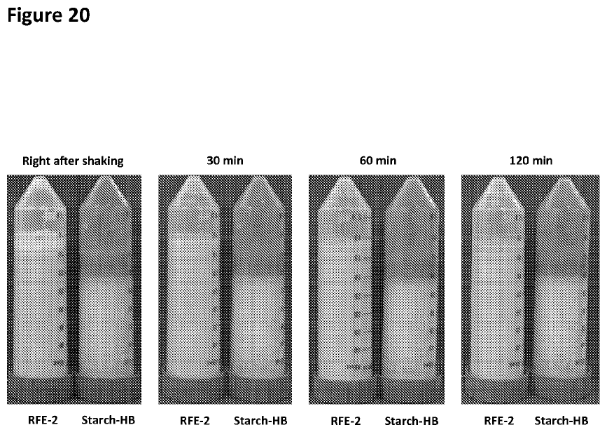

FIG. 20 shows the images of homogenized mixtures taken right after shaking (within 2 min after shaking) and at 30, 60, and 120 min after shaking. It is shown that at any point after shaking, Starch-HB was not able to form emulsion. Even right after the shaking, the oil-water separation occurred immediately. This shows that the emulsification capability of Starch-HB was negligible. In contrast, RFE-2 stabilized the emulsion all through 120 min after shaking, highlighting the importance of protein components in the flour for providing acceptable emulsification properties.

Therefore, while a combined heating and milling treatment of flour leads to the production of flour emulsifier, such a combined treatment does not yield an acceptable emulsifier from isolated starch (with protein removed).

### Example 21. Use of Flour Emulsifier in Cake Making

Rice flour emulsifiers prepared using a combined heating and milling treatment were used in the cake formulation, with a goal to fully replace conventional emulsifiers and gums, as well as to partially replace egg.

The formulations of standard cake, negative control cake (without added emulsifiers and gums and with reduced amount of egg), and rice flour enhanced cake are listed below:
Standard cake: cake flour 200 g, sugar 240 g, fresh egg 240 g, shortening 120 g, baking powder 1 g, salt 1 g, SSL (sodium stearoyl lactylate) 0.2 g, lecithin 0.6 g, guar gum 0.2 g, xanthan gum 0.2 g, and water 40 g
Negative control cake: cake flour 200 g, sugar 240 g, fresh egg 204 g (a 36 g, or 12.5% reduction), shortening 120 g, baking powder 1 g, salt 1 g, SSL (sodium stearoyl lactylate) 0 g (complete removal), lecithin 0 g (complete removal), guar gum 0 g (complete removal), xanthan gum 0 g (complete removal), and water 40 g
Rice flour enhanced cake: cake flour 200 g, sugar 240 g, fresh egg 204 g (a 36 g, or 12.5% reduction), shortening 120 g, baking powder 1 g, salt 1 g, SSL (sodium stearoyl lactylate) 0 g (complete removal), lecithin 0 g (complete removal), guar gum 0 g (complete removal), xanthan gum 0 g (complete removal), rice flour emulsifier 11.4 g, and water 67 g (water increased to compensate for the moisture loss due to the reduction of fresh egg).
In the rice flour enhanced cake, the rice flour emulsifier added provided multiple functionalities that included emulsification, viscosity increase, and bulking.
   Among the cakes prepared using 3 different formulations, the sensory panel indicated the following results:
Rice flour enhanced cake showed the best texture, sensory quality, including the right amount of moistness, softness, and fluffiness, and prolonged refrigeration shelf life.
Rice flour enhanced cake had similar volume as standard cake.
Negative control cake had worst texture and mouth feel (too dry and fluffy).

### Example 22. Use of Flour Emulsifier for Encapsulation of Oils

In this example, the capability of flour emulsifier to form encapsulation with oil was demonstrated. A rice flour emulsifier (RFE) subjected to combined heating and milling was tested as carrier, along with gum arabic and octenylsuccinate starch (OSA-starch) as controls. The oil used was orange oil. The procedure is as following:

The carriers (i.e. RFE, gum arabic, OSA-starch) was dissolved in 0.02 mM sodium acetate (NaAc) buffer to form 15% (w/v) dispersion. Orange oil was added to the dispersion in a ratio of oil/carrier 1:3. The pH was adjusted to around 7.0.
The coarse oil-in-water emulsions were prepared using a food processor at room temperature. Thereafter, the coarse emulsion was subjected to a high-pressure homogenizer (Panda, GEA) for 5 passes at around 8000-9000 psi. The fine emulsions obtained were spray-dried using a Büchi spray dryer (B-290, Büchi). Operating conditions were: inlet temperature at 125-130° C., outlet temperature at 80-90° C., and feed rate at 6 mL/min. The solids in powder form were collected as the encapsulated products of orange oil. The RFE-orange oil encapsulation product dispersed in water rapidly to form an emulsion.

### Example 23. Use of Flour Emulsifier in the Preparation of Coffee Creamer

115 g water and 15 g flour emulsifier were mixed and the mixture was homogenized for 10 times, each time for 30 seconds using a food processor. To the dispersion, 15 g coconut oil was added and the mixture was further homogenized for 20 times, each time for 15 seconds to form the emulsion. To the emulsion, 0.72 g dipotassium phosphate, 1 g vanilla extract, and 5 g Great value TM sweetener (containing sucralose) was added. The mixture thus obtained was flour emulsifier-containing creamer. Coffee was prepared by dispersing 2 g NESCAFÉ Clásico™ dark roast instant coffee powder in 180 g water. To each cup of coffee, 15 g flour emulsifier-containing creamer was added. As a comparison, the coffee was added with a commercial creamer (International Delight French Vanilla Creamer Singles).

The result showed that the creamer formulated with flour emulsifier was comparable with commercial creamer with regard to the whitening effect, the effect to mask coffee acidity, and the effect to offer smoothness and creaminess of the coffee drink.

### Example 24. Use of Flour Emulsifier in the Preparation of Muffin

Muffins were prepared using the ingredients listed in Table 4. Sugar, shorting, and egg were mixed for 2 min for creaming. Thereafter, other ingredients were added and mixed for 1.5 minutes. In each muffin cup, 60 g batter was loaded and baked at 425° F. for 23 min. The appearance and sensory qualities of muffins were compared.

TABLE 4
Formulations to prepare muffins

| | Negative | Positive | |
|---|---|---|---|
| Ingredient | Control | control | Flour emulsifier- |
| (grams) | (gram) | (gram) | containing (gram) |
| Pastry flour | 110 | 100 | 100 |
| Sugar | 60 | 60 | 60 |
| Baking powder | 5 | 5 | 5 |
| Salt | 1.25 | 1.25 | 1.25 |
| Nonfat dry milk | 7.5 | 7.5 | 7.5 |
| Shortening | 40 | 40 | 40 |
| Egg | 30 | 30 | 30 |
| Water | 110 | 110 | 110 |
| Monoglyceride | — | 1.5 | — |
| Propylene glycol | — | 2 | — |
| monostearate | | | |
| Sodium stearoyl | — | 0.25 | — |
| lactylate | | | |
| Xanthan gum | — | 0.6 | — |
| Guar gum | — | 0.6 | — |
| Flour emulsifier | — | — | 10 |

The result showed that the muffins made without using emulsifiers and gums (negative control) were smallest among the three formulations. The muffins formulated with flour emulsifier showed better dome shape than muffins formulated with gums and synthetic emulsifiers (positive control).

The sensory qualities of muffins formulated with flour emulsifier was better than that of negative control and positive control. The flour emulsifier-formulated muffin was moist and tender after refrigeration storage. In contrast, the negative control was dry and crumbly, and the positive control was very gummy and sticky.

### Example 25. Use of Flour Emulsifier in the Preparation of Frozen Dessert

Frozen desserts were prepared using the ingredients listed in Table 5. First, ingredients except for the oil were dispersed in water and the dispersion was homogenized for 10 times, each time for 30 seconds using a food processor. Thereafter, oil was added to the dispersion and the mixture was heated at 75° C. for 45 minutes. Then, the mixture was homogenized for 20 times, each time for 15 seconds. Thereafter, the mixture (emulsion) formed was cooled to the room temperature and then chilled overnight in a refrigerator. After chilling, the emulsion was processed using an ice cream maker (Whynter ICM-200LS) and the material obtained was hardened in a freezer to yield frozen dessert product.

TABLE 5
Formulations to prepare frozen desserts

| | | | Flour |
|---|---|---|---|
| | Negative | Positive | emulsifier- |
| | control | control | containing |
| Ingredient | (gram) | (gram) | (gram) |
| Sugar | 20 | 20 | 20 |
| Lecithin | 0 | 1 | 0 |
| Guar gum | 0 | 0.07 | 0 |
| Locust bean gum | 0 | 0.07 | 0 |
| Carrageenan | 0 | 0.07 | 0 |
| Maltodextrin M180 | 5 | 3.8 | 0 |
| Flour emulsifier | 0 | 0 | 3 |
| Mono- and diglycerides | 0 | 0.1 | 0 |
| Coconut/vegetable oil | 15 | 15 | 15 |
| (75/25, v/v) | | | |
| Water | 60 | 59.9 | 62 |

The sensory properties of frozen dessert formulated with flour emulsifier was comparable with that of positive control (froze dessert formulated with gums, lecithin, and mono- and diglycerides) and better than that of negative control (without gums and emulsifiers). For the negative control, it was very hard to scoop due to the rigid texture and tasted very icy. In comparison, the positive control showed smooth and fluffy texture, was easy to scoop, and tasted very creamy. For the frozen dessert formulated with flour emulsifier, it showed comparable smoothness, fluffiness, easy-to-scoop, and creaminess with the positive control.

### Example 26. Use of Flour Emulsifier in the Preparation of Tortillas

The formulations of tortilla are listed in Table 6. Flour, other dry ingredients, and shortening were mixed for 3 minutes. Thereafter, water was added and the dough was further mixed for 6 minutes. The dough obtained was divided, rounded, proofed, hot pressed, and baked. The tortillas produced were then cooled, stacked, and packaged in plastic bags.

The tortillas prepared using flour emulsifier showed similar performances in processing, taste, flexibility, appearance, texture, and non-stickiness to that of control (i.e. tortillas prepared using guar gum and mono- and diglycerides).

TABLE 6
Formulations to prepare tortillas

| | | Flour emulsifier- |
|---|---|---|
| | Control | containing |
| Ingredients/Product | (Gram) | (Gram) |
| Salt | 13.5 | 13.5 |
| Calcium propionate | 7.2 | 7.2 |
| Fumaric acid | 3.15 | 3.15 |
| Potassium sorbate | 3.15 | 3.15 |
| Sodium bicarbonate | 8.1 | 8.1 |
| Sodium aluminium sulfate | 8.1 | 8.1 |
| Mono-calcium phosphate | 1.35 | 1.35 |
| Guar gum | 2.25 | 0 |
| Sodium metabisulfite | 0.04 | 0.04 |
| Mono- and diglycerides | 6.3 | 0 |
| Flour emulsifier | 0 | 8.55 |
| Wheat Flour | 900 | 900 |
| Shortening | 90 | 90 |
| Water | 480 | 480 |

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### Example 27: Preparation of Flour Emulsifier

Polished rice grain was firstly milled using a hammer mill to pass an 80-mesh sieve and the generated rice flour was subjected to heating and milling. The heating temperature was 110° C. with a total time of 150 minutes, and the milling was conducted for a total of 120 minutes with a power input of 0.7 kw to 1.2 kw per kg of rice flour. The material collected after the heating and milling process was a flour emulsifier termed as “FE1”. The FE1 material was used in Examples 28 to 35.

### Example 28: Freeze-Thaw Stability of Emulsion Containing FE1

Preparation of emulsion: Five grams of FE1 was dispersed in 85 mL of distilled water and the dispersion was subjected to 5 times of blending using a food processor (SMASH BLEND 14, Oster, Hilliard, Ohio) with each time for 30 seconds. Thereafter, 10 grams of oil (vegetable oil containing 3% of paprika oleoresin) was added to the dispersion, and the mixture was homogenized for 10 times, each time for 15 seconds using a food processor (SMASH BLEND 14, Oster).

Freeze-thaw testing: The emulsion formed was added to a 50-mL tube and then subjected to 5 cycles of freeze-thaw treatment. Each freeze-thaw cycle comprised a step of placing the tube at −20° C. for 21 hours and a step of placing the tube at 30° C. for 3 hours. The changes of emulsion appearance over the freeze-thaw treatment were photographed. Emulsion formed using a commercial octenylsuccinate starch (OSA-starch) was used as comparison.

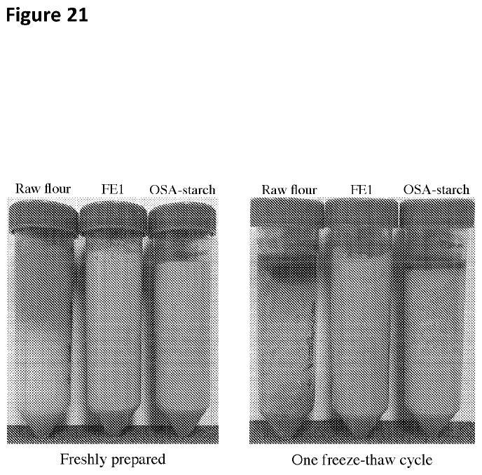

Result: As shown in FIG. 21, both FE1 and OSA-starch were able to form emulsions. In contrast, rice flour not subjected to heating and milling could not form emulsion effectively. For emulsions subjected to freeze-thaw treatment, emulsion formed with OSA-starch showed a thick oil layer, whereas no noticeable oil layer was found with emulsion formed with FE1. The result indicated that the freeze-thaw stability of emulsion formed with FE1 was much higher than the emulsion formed with regular rice flour and the emulsion formed with OSA-starch.

### Example 29: Particle Size of Emulsions Formed Using FE1

Preparation of emulsion: Formulations of emulsions are shown in Table 8. Thirty grams of FE1 were dispersed in 510, 540, or 555 grams of distilled water. To each dispersion, 60, 30, or 15 grams of vegetable oil were added and the mixture was

| | | | | Percentage | Percentage |
|---|---|---|---|---|---|
| | | | | of particles | of particles |
| | FE1 | Oil | | with size | with size |
| | con- | con- | Z- | (diameter) | (diameter) |
| Emul- | tent, | tent, | average | greater | greater |
| sion | % | % | size, nm | than 5 μm, % | than 10 μm, % |
| 1 | 5 | 2.5 | 734.5 ± 13.6 | <1 | <0.5 |
| 2 | 5 | 5 | 840.8 ± 37.9 | <0.5 | <0.5 |
| 3 | 5 | 10 | 977.0 ± 85.6 | <0.5 | <0.5 |

first homogenized for 10 times, each time for 15 seconds using a food processor (SMASH BLEND 14, Oster) and then further homogenized using a high-pressure homogenizer (PandaPlus, GEA, Italy) at 2500 psi for 2 passes to generate emulsion. The particle size values of emulsions were determined using Zetasizer Nano (ZS90, Malvern Instruments) at 25° C.

Result: The particle size properties of emulsions are shown in Table 8. The Z-average particle size with Emulsion 1, 2, and 3 were 734.5, 840.8, and 977.0 nm, respectively. The percentages of particles with size greater than 5 μm were <1%, <0.5%, and <0.5% for emulsion 1, 2, and 3, respectively.

Table 8. Formulations and particle size properties of emulsions

### Example 30: Use of FE1 to Prepare Coffee Creamer

Coffee creamer preparation: In 97.6 grams of distilled water, 13.4 grams of FE1, 66.6 grams of sucrose, 1 gram of dipotassium phosphate, and 2 grams of vanilla liquid were dispersed. To the dispersion, 20 grams of palm oil were added and the mixture was homogenized for 10 times, each time for 15 seconds using a food processor (SMASH BLEND 14, Oster). The emulsion formed was collected as the coffee creamer.

Test of coffee creamer: Black coffee drink was prepared through dispersing 2 grams NESCAFE Clásico™ dark roast instant coffee powder in 180 grams of hot water. To one cup (182 grams) of black coffee drink, 15 grams of creamer was added. The black coffee and the coffee drink added with a commercial creamer (International Delight French Vanilla Creamer Singles) were used as references. The organoleptic properties including whiteness, acidity, and bitterness were evaluated by panelists.

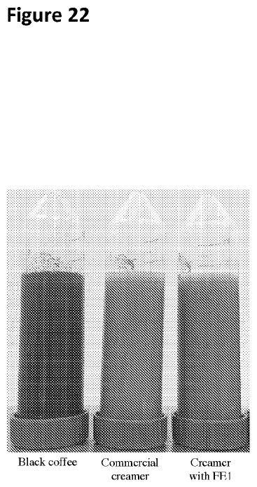

Result: As shown in FIG. 22, the coffee creamer prepared using flour emulsifier FE1 showed essentially the same whitening effect as that of the commercial creamer. Based on the sensory tests, both FE1-containing creamer and commercial creamer performed similarly in providing creamy mouthfeel and masking the bitterness and acidity originally in black coffee.

### Example 31: Almond Milk Prepared Using FE1

Preparation of crude almond milk: 100 grams of raw almond from a local grocery store were added in 300 grams of distilled water. After 15 hours of soaking, the mixture was ground using a food processor (MX1100XTX Xtreme, Waring). The slurry generated was passed through a 140-mesh sieve, the retentate was blended with additional 300 grams of water, and the slurry was again passed through a 140-mesh sieve. The filtrates were combined as crude almond milk.

Further processing of crude almond milk: 10 grams of FE1 was dispersed in 90 grams of water. The dispersion was then mixed with 100 grams of crude almond milk and the mixture was homogenized using a food processor (SMASH BLEND 14, Oster) for 10 times, each time for 15 seconds. The emulsion generated was mixed with 800 grams of water to yield the almond milk. As a reference, 100 grams of crude almond milk was mixed with 100 grams of water, the mixture was homogenized, and the emulsion was further mixed with 800 grams of water to yield the almond milk without PE1.

Observation of oil droplets in almond milk: The oil droplet of almond milk was observed using a Compound Monocular Microscope (Boreal, N.Y.).

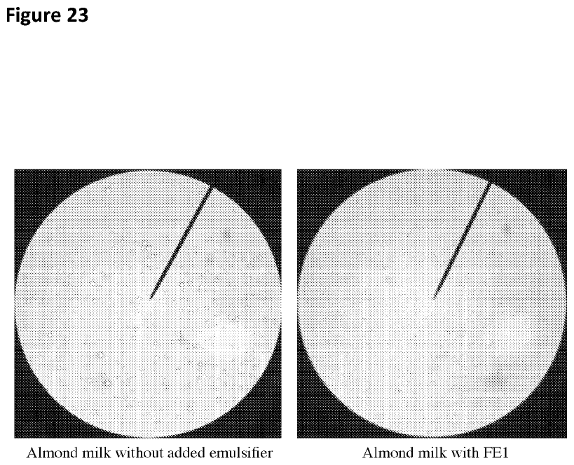

Result: As shown in FIG. 23, the almond milk with FE1 showed much smaller oil droplets than the almond milk without added emulsifier, suggesting the capability of FE1 to stabilize almond milk as an emulsion.

### Example 32: Black Pepper Oleoresin Emulsified Using FE1

Preparation of emulsion of black pepper oleoresin: In 78 grams of water, 22 grams of sodium chloride was added to make a 22% salt solution. To 83.3 grams of this salt solution, 10 grams of FE1 was dispersed. To the dispersion generated, 6.7 grams of black pepper oleoresin was added, and the mixture was homogenized using a food processor (SMASH BLEND 14, Oster) for 10 times, each time for 15 seconds to yield the emulsion of black pepper oleoresin. As a reference, 6.7 grams of black pepper oleoresin were mixed with 93.3 grams of salt solution and the mixture was homogenized using the same procedure described above to yield an emulsion without PE1. Both emulsions were added in 50-mL tubes for comparison. Photographs were taken after shaking both tubes at the same time for 20 times.

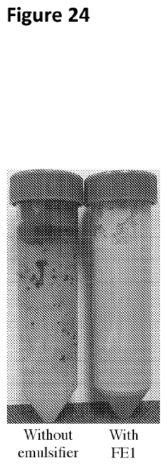

Result: As shown in FIG. 24, a stable emulsion of black pepper oleoresin was formed using FE1 as emulsifier.

### Example 33: Jojoba Oil Cream Prepared Using FE1

Preparation of jojoba oil cream: Three grams of FE1 was dispersed in 75.8 grams of water. To the PE1 dispersion, 20 grams of jojoba oil was added, and the mixture was homogenized using a food processor (SMASH BLEND 14, Oster) for a total of 150 seconds to yield the emulsion. As a reference, 20 grams of jojoba oil was mixed with 78.8 grams of water and the mixture was homogenized for a total of 150 seconds to yield the reference emulsion. To each emulsion (98.8 grams), 0.6 grams of guar gum and 0.6 grams of xanthan gum were added, the mixture was homogenized using a food processor for 30 seconds and then agitated for 1 hour in a shaking water bath (50° C., 100 rpm). The products (jojoba cream with or without PE1) collected were each added to a 1.5-mL centrifuge tube and subjected to centrifugation (3000×g, 30 minutes).

Result: For jojoba cream without emulsifier, centrifugation resulted in a noticeable oil layer on the top of the cream. In contrast, for jojoba cream with PE1, centrifugation did not lead to the formation of visible oil layer, suggesting a long-term storage stability of this jojoba oil cream.

### Example 34: French Dressing Prepared Using FE1

Preparation of French dressing: 10 grams of FE1, 2 grams of salt, and 18 grams of vinegar were dispersed in 94 grams distilled water. To the dispersion, 76 grams of oil (vegetable oil containing 3% of paprika oleoresin) were added and the mixture was homogenized using a food processor for 10 times, each time for 15 seconds to yield the emulsion as the French dressing. To prepare reference dressing, rice flour not subjected to heating and milling (raw flour) was used to prepare emulsion using the same procedure as described above.

Observation of oil droplets in French dressing: French dressings were diluted for 40 times with water and observed using a Compound Monocular Microscope.

Result: As shown in FIG. 25, the dressing made with raw flour showed layer separation, whereas the dressing made with FE1 was uniform and stable. In addition, the oil droplets of dressing formed with FE1 were much smaller than those of dressing formed with raw flour. Such a result indicates that FE1 is an effective emulsifier for preparing French dressing.

### Example 35: Viscosity of FE1 Dispersion

Preparation of FE1 dispersions: The 5%, 10%, 20%, and 30% (w/w) FE1 dispersions were prepared through dispersing 10 grams of FE1 in 190 grams of water, 20 grams of FE1 in 180 grams of water, 40 grams of FE1 in 160 grams of water, and 75 grams of FE1 in 175 grams of water, respectively. Each FE1 dispersion was prepared using an KitchenAid mixer (speed 2, 15 min).

Viscosity measurement: Each FE1 dispersion was transferred to a cup, and its viscosity was measured using a digital viscometer (DV-1, WANT).

Result: The viscosity of 5%, 10%, 20%, and 30% (w/w) FE1 dispersions were 191.3, 464.3, 5378, and 33271 mPa·s, respectively.

### Additional Embodiments

In addition to the foregoing disclosure, there are provided the following embodiments.

Embodiment 1. An emulsifier composition originated from a plant material comprising about 1% to 85% protein and about 15% to 99% carbohydrate, wherein said carbohydrate includes starch component.

Embodiment 2. The emulsifier composition of embodiment 1, wherein said plant material is selected from flour, meal, fraction or whole grain of cereal grain, legume, tuber, root, stem, seed, nut of a plant, and the combination thereof.

Embodiment 3. The emulsifier composition of embodiment 1, wherein the crystallinity of the starch component in said emulsifier composition is about 90% or less of the crystallinity of the starch component of the original plant material.

Embodiment 4. The emulsifier composition of embodiment 1, wherein said emulsifier composition is able to form an emulsion.

Embodiment 5. The emulsifier composition of embodiment 1, wherein said emulsifier composition is a flour originated from a plant material, wherein said plant material comprises one or more selected from wheat, corn, rice, wild rice, barley, fonio, Job's tears, sorghum, millet, oats, rye, teff, triticale, buckwheat, tartary buckwheat, amaranth, quinoa, pitseed goosefoot, canihua, chia, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, arhar/toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taro, tuber, cassava (tapioca), water chestnut, arrowroot, sweet potato, Chinese yam, lotus root, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, and the combination thereof.

Embodiment 6. The emulsifier composition of embodiment 1 is a flour of one or a mixture of cereal grains, legumes, tubers, roots, stems, seeds, nuts, and other plant materials, wherein the emulsifier composition is able to form an emulsion that contains an oil phase and wherein said emulsion has emulsifier composition-to-oil ratio of about 1/100 to about 100/1, preferentially of about 1/10 to about 10/1, wherein the oil phase optionally comprises additional compounds.

Embodiment 7. The emulsifier composition of embodiment 6, wherein the emulsion is characterized with at least one of following features:
a. the average size (diameter) of the oil droplets in said emulsion is not greater than 1000 μm, 500 μm, 100 μm, 50 μm, 30 μm, 10 μm, 5 μm, 1 μm, 0.5 μm, 0.2 μm, 0.1 μm, or 0.05 μm; or
b. the portion (by volume, weight, or number) of the oil droplets with size (diameter) greater than 500 μm is not greater than 95%, 90%, 80%, 70%, 60%, 50%, 40%, 30%, 20%, 10%, 5%, 2%, or 1%; or
c. the portion (by volume, weight, or number) of the oil droplets with size (diameter) greater than 50 μm is not greater than 95%, 90%, 80%, 70%, 60%, 50%, 40%, 30%, 20%, 10%, 5%, 2%, or 1%; or
d. the portion (by volume, weight, or number) of the oil droplets with size (diameter) greater than 10 μm is not greater than 95%, 90%, 80%, 70%, 60%, 50%, 40%, 30%, 20%, 10%, 5%, 2%, or 1%; or
e. the portion (by volume, weight, or number) of the oil droplets with size (diameter) greater than Sum is not greater than 95%, 90%, 80%, 70%, 60%, 50%, 40%, 30%, 20%, 10%, 5%, 2%, or 1%; or
f. the portion (by volume, weight, or number) of the oil droplets with size (diameter) greater than 1 μm is not greater than 95%, 90%, 80%, 70%, 60%, 50%, 40%, 30%, 20%, 10%, 5%, 2%, or 1%; or
g. under 1×g force (e.g. regular gravity), the visible separation in the emulsion, including but not limited to creaming, flocculation, aggregation, sedimentation, and precipitation, occurs at least after 0.1 min, 0.5 min, 1 min, 5 min, 10 min, 20 min, 50 min, 100 min, 200 min, 500 min, 1000 min, 2000 min, 5000 min, 10,000 min, 20,000 min, 50,000 min, or 100,000 min; or
h. after subjecting the emulsion to centrifugation for about 1 minute, the visible separation in the emulsion, including but not limited to creaming, flocculation, aggregation, sedimentation and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g; or
i. after subjecting the emulsion to centrifugation for 10 minute, the visible separation in the emulsion, including but not limited to creaming, flocculation, aggregation, sedimentation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g; or
j. the emulsion containing said emulsifier composition is at least 1 time, 2 times, 3 times, 4 times, 5 times, 6 times, 7 times, 8 times, 9 times, or 10 times more stable than the emulsion containing same amount of regular flour from the same plant origin.

Embodiment 8. The emulsifier composition according to embodiment 6, wherein said emulsifier composition provides protection to the oil phase or compounds in the oil phase against photo instability, oxidation, chemical instability, volatility, pH instability, temperature instability, or color instability, taste change, flavor change, etc.

Embodiment 9. A product comprising the emulsifier composition of embodiment 1, wherein said product is food, food ingredient, food additive, beverage, personal care ingredient or product, cosmetics ingredient or product, medical product, drug excipient, drug, industrial product, agricultural product, or other product alike.

Embodiment 10. A product comprising the emulsifier composition of claim 1, wherein said product is a oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, animal feed, feed additive, pet food, fish feed, fragrance, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, fabric or leather treatment chemical, plastic, rubber, container, utensil, packaging, tire, cloth, fabric, leather, or an ingredient to any of above products.

Embodiment 11. The product according to any of embodiments 9-10, wherein the emulsifier composition provides thickening, texture improving, creaminess, improved mouthfeel, improved freeze-thaw stability, improved physical stability, and/or other properties to an emulsion or a product.

Embodiment 12. An emulsifier composition comprising a flour made from a plant material selected from flour, meal, fraction or whole grain of a cereal grain, legume, tuber, root, stem, seed, nut, and the combination thereof, wherein the emulsifier composition contains both protein and carbohydrate including starch, wherein the content of protein is about 1% to 85% of said emulsifier composition, wherein the content of carbohydrate is about 15% to 99% of said emulsifier composition and wherein the crystallinity of said starch in said emulsifier composition is less than 90% of the crystallinity of the starch in the original plant material.

Embodiment 13. A process for preparing an emulsifier, wherein said emulsifier comprises at least a protein component ranging from about 1% to about 85% of said emulsifier, and a carbohydrate component ranging from about 15% to about 99% of said emulsifier, wherein said process comprises one or more steps of milling and heating an original plant material selected from at least one of flour, meal, fraction or whole grain of cereal grain, legume, potato, yam, taco, tuber, root, stem, nut, seed, and the combination thereof, and wherein the carbohydrate component contains starch.

Embodiment 14. The process for producing the emulsifier according to embodiment 13, wherein the crystallinity of the starch in said emulsifier is less than 90% of the crystallinity of the starch in the original plant material.

Embodiment 15. The process for producing the emulsifier according to embodiment 13, wherein the crystallinity of said starch in said emulsifier is less than 85% of the crystallinity of the starch in the original plant material.

Embodiment 16. The process for producing the emulsifier according to embodiment 13, wherein said milling process uses at least one form of mill selected from hammer mill, ball mill, jet mill, stone mill, roller mill, stirred mill, stirred ball mill, colloidal mill, attritor, homogenizer, fluidizer, high speed blender, sigma blender, or extruder.

Embodiment 17. The process for producing the emulsifier according to embodiment 13, wherein said milling process lasts about 2 minutes to about 50 hours.

Embodiment 18. The process for producing the emulsifier according to embodiment 13, wherein at least one of said milling process has a power input of not less than 0.05 kilowatts (kw) per kilogram (kg) of said plant material.

Embodiment 19. The process for producing the emulsifier according to embodiment 13, wherein at least one of said milling process has a power input of not less than 0.1 kw per kg of said plant material.

Embodiment 20. The process for producing the emulsifier according to embodiment 13, wherein at least one of said milling process has a power input of not less than 0.2 kw per kg of said plant material.

Embodiment 21. The process for producing the emulsifier according to embodiment 13, wherein at least one of said milling process has a power input of not less than 0.5 kw per kg of said plant material.

Embodiment 22. The process for producing the emulsifier according to embodiment 13, wherein said heating process uses at least one method or facility of heating selected from oven, vacuum oven, ventilated oven, microwave oven, near infrared oven, steaming, hot gas heating, container with jacket for heating, static heating, stirred heating, jet cooking, temperature regulator or controller, dryer, heat tunnel, heat tube, or heat exchanger.

Embodiment 23. The process for producing the emulsifier according to embodiment 13, wherein said heating process has a temperature from about 40° C. to about 300° C., a stepwise gradient thereof, or a combination of different temperature thereof, for a period of about 2 minutes to about 100 hours.

Embodiment 24. The process for producing the emulsifier according to embodiment 13, wherein said milling process and heating process take no particular order.

Embodiment 25. The process for producing the emulsifier according embodiment 13, wherein said milling process and heating process are combined into a single step of milling under elevated temperature.

Embodiment 26. The process for producing the emulsifier according to embodiment 13, wherein said plant material is one or a mixture of plant materials from wheat, corn, rice, wild rice, barley, fonio, Job's tears, sorghum, millet, oats, rye, teff, triticale, buckwheat, tartary buckwheat, amaranth, quinoa, pitseed goosefoot, canihua, chia, alfalfa, clover, peas, beans, chickpeas, lentils, lupin bean, mesquite, carob, soybeans, peanuts, tamarind, kidney bean, navy bean, pinto bean, hericot bean, lima bean, butter bean, adzuki bean, azuki bean, mung bean, golden gram, green gram, black gram, urad, scarlet runner bean, ricebean, moth bean, tepary bean, horse bean, broad bean, field bean, garden pea, protein pea, chickpea, cowpea, black-eyed pea, blackeye bean, pigeon pea, Arhar/Toor, cajan pea, Congo bean, gandules, Bambara groundnut, earth pea, vetch, common vetch, lupins, lablab, hyacinth bean, jack bean, sword bean, winged bean, vevet bean, cowitch, yam bean, potato, yam, taro, tuber, cassava (tapioca), water chestnut, arrowroot, sweet potato, Chinese yam, lotus root, almond, cashew, chestnut, coconut, hazelnut, macadamia, peanut, pecan, pine nut, pistachio, walnut, betel nut, kola nut, brazil nut, sesame seed, ginko nut, bread nut, jack nut, acorn, beech, or the combination thereof.

Embodiment 27. The process for producing the emulsifier according to embodiment 13, further comprising a step of hydrothermal treatment of said plant material, including at least one method selected from cooking, annealing, gelatinizing, steaming, baking, microwaving, extruding, and homogenizing said plant material.

Embodiment 28. The process for producing the emulsifier according to embodiment 13, further comprising a step of drying of said plant material after subjecting it to hydrothermal treatment including at least one method selected from cooking, annealing, gelatinizing, steaming, baking, microwaving, extruding, fluid bed treatment, granulation, and homogenizing said plant material.

Embodiment 29. A process for preparing an emulsifier from a plant material selected from flour, meal, fraction or whole grain of cereal grains, legumes, a tuber, root, stem, seed, nut, and a combination thereof, comprising one or more steps of milling process and heating process of said plant material to afford an emulsifier,
wherein each said milling process lasts about 2 minutes to about 50 hours;
wherein at least one of said milling processes has a power input no less than about 0.05 kw per kg of said plant material; and
wherein each said heating process has a temperature ranging from about 40° C. to about 300° C. for a period of about 2 minutes to about 100 hours.

Embodiment 30. A process for preparing an emulsifier from an original plant material selected from flour, meal, fraction or whole grain of cereal grains, legume, tuber, root, stem, seed, nut, and the combination thereof, comprising one or more steps of milling process and heating process of said plant material to afford an emulsifier, wherein:
a. each said milling process lasts about 2 minutes to about 50 hours;
b. at least one of said milling processes has a power input no less than about 0.05 kw per kg of said plant material;
c. each said heating process has a temperature from about 40° C. to about 300° C. for a period of about 2 minutes to about 100 hours;
d. said emulsifier contains both protein and carbohydrate component including starch, wherein the content of said protein is 1% to 85% of the said emulsifier, the content of carbohydrate is about 15% to about 99% of the said emulsifier; and the crystallinity of said starch in said emulsifier is less than 90% of the crystallinity of the starch in the original plant material.

Embodiment 31. An emulsifier prepared according to the process of any of embodiments 13-30.

Embodiment 32. The emulsifier of embodiment 31, wherein the emulsifier can form an emulsion.

Embodiment 33. The emulsifier of embodiment 32, wherein the emulsion comprises an emulsifier-to-oil ratio of about 1/100 to about 100/1, preferentially of about 1/10 to about 10/1, wherein the emulsion is characterized with at least one of following features:
a. the average size (diameter) of the oil droplets in said emulsion is not greater than 1000 μm, 500 μm, 100 μm, 50 μm, 30 μm, 10 μm, 5 μm, 1 μm, 0.5 μm, 0.2 μm, 0.1 μm, or 0.05 μm; or
b. the portion (by volume, weight, or number) of the oil droplets with size (diameter) greater than 500 μm is not greater than 95%, 90%, 80%, 70%, 60%, 50%, 40%, 30%, 20%, 10%, 5%, 2%, or 1%; or
c. the portion (by volume, weight, or number) of the oil droplets with size (diameter) greater than 50 μm is not greater than 95%, 90%, 80%, 70%, 60%, 50%, 40%, 30%, 20%, 10%, 5%, 2%, or 1%; or
d. the portion (by volume, weight, or number) of the oil droplets with size (diameter) greater than 10 μm is not greater than 95%, 90%, 80%, 70%, 60%, 50%, 40%, 30%, 20%, 10%, 5%, 2%, or 1%; or
e. the portion (by volume, weight, or number) of the oil droplets with size (diameter) greater than Sum is not greater than 95%, 90%, 80%, 70%, 60%, 50%, 40%, 30%, 20%, 10%, 5%, 2%, or 1%; or
f. the portion (by volume, weight, or number) of the oil droplets with size (diameter) greater than 1 μm is not greater than 95%, 90%, 80%, 70%, 60%, 50%, 40%, 30%, 20%, 10%, 5%, 2%, or 1%; or
g. under 1×g force (e.g. regular gravity), the visible separation in the emulsion, including but not limited to creaming, flocculation, aggregation, sedimentation, and precipitation, occurs at least after 0.1 min, 0.5 min, 1 min, 5 min, 10 min, 20 min, 50 min, 100 min, 200 min, 500 min, 1000 min, 2000 min, 5000 min, 10,000 min, 20,000 min, 50,000 min, or 100,000 min; or
h. after subjecting the emulsion to centrifugation for about 1 minute, the visible separation in the emulsion, including but not limited to creaming, flocculation, aggregation, sedimentation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g; or
i. after subjecting the emulsion to centrifugation for 10 minute, the visible separation in the emulsion, including but not limited to creaming, flocculation, aggregation, sedimentation, and precipitation, occurs at a centrifugation force of at least 1×g, 2×g, 5×g, 10×g, 20×g, 50×g, 100×g, 200×g, 500×g, 1000×g, 2000×g, 5000×g, 10,000×g, 20,000×g, and 50,000×g.
j. The emulsion containing said emulsifier composition is at least 1 time, 2 times, 3 times, 4 times, 5 times, 6 times, 7 times, 8 times, 9 times, or 10 times more stable than the emulsion containing same amount of regular flour from the same plant origin.

Embodiment 34. An emulsion comprising an emulsifier of any of embodiments 1-12, or 31.

Embodiment 35. The emulsion of embodiment 34 comprising an aqueous phase and an oil phase,
a. wherein the oil phase of said emulsion comprises a lipophilic component or a plurality of lipophilic components, and
b. wherein the aqueous phase of said emulsion contains water or a combination of water and one or a plurality of non-water components.

Embodiment 36. The emulsion of embodiment 35, wherein said lipophilic component includes but is not limited to oil, fat, butter, other emulsifier, active pharmaceutical ingredient, pharmaceutical excipient, biocide, herbicide, pesticide, hormone or plant hormone, plant nutrients, fertilizer, plant protection ingredient, agricultural chemical, agricultural carrier, preservative, flavor or fragrant, nutrients, vitamin, nutrient, color, natural extract, antibody, antibiotics, antimicrobial, food additive or ingredient, supplement additive or ingredients, cosmetic additive or ingredient, additive or ingredient for personal care products, agricultural additive or ingredient, medical additive or ingredients, and industrial products.

Embodiment 37. The emulsion of embodiment 35, wherein said aqueous phase is a solution, suspension, or a mixture of sugar, salt, protein, peptide, flavor, color, vitamin, antioxidant, antimicrobial compound, fragrant, antibody, enzyme, active pharmaceutical ingredient, pharmaceutical excipient, fertilizer, plant hormone, plant nutrient, agricultural chemical, agricultural carrier, herbicide, pesticide, nutrient, food additive and ingredient, cosmetic additive and ingredient, additive or ingredient for personal care products, stabilizer, or emulsifier.

Embodiment 38. The emulsion of embodiment 34, wherein said emulsion is further processed to products, including but not limited to a food, beverage, food ingredient, pharmaceutical product for human or animal consumption, feed product, plant protection, product for agricultural industry, product for personal care, personal hygiene, including lotion, cream, shampoo, conditioner, soap, industrial product, and the like.

Embodiment 39. The emulsion of embodiment 38, wherein said products include but are not limited to oleoresin, essential oil, encapsulation, protein shake, smoothie, cake, muffin, donut, tortilla, bread, flat bread, chip, cracker, cookie, pie, bar, batter, dough, baked goods, frozen dough, icing, topping, filling, ice cream, frozen food, frozen dessert, condiment or culinary food, soup, sauce, dressing, gravy, food entry, coffee creamer, dried or liquid color formulation, dried or liquid flavor formulation, dried or liquid nutrients formulation, meat product, plant meat alternative products, brine, dried powder food or animal feed, dairy, milk alternative, protein drink, energy drink, beverage, yoghurt, meal replacer, plant protein drink, flavor additive, color additive, nutrition additive, supplement, fragrance, marijuana or cannabis products allowed by law, pharmaceutical product for human or animal consumption, product for agricultural industry, cream, lotion, skincare products, foundation, powders, shampoo, conditioner, soap, detergent, dish cleanser, household or industrial cleaning supply, plastic, rubber, industrial coating, paint, fabric or leather coating or treatment liquid, construction material, and the like, or the ingredients or combinations of any of above products.

Embodiment 40. An encapsulation composition contains an emulsifier of any of embodiments 1-12, or 31.

Embodiment 41. An encapsulation composition made through the dehydration of the emulsion in claim 35.

Embodiment 42. The encapsulation composition of embodiment 41, wherein
a. said lipophilic component is at least one selected from an oil, fat, butter, other emulsifier, active pharmaceutical ingredient, pharmaceutical excipient, biocide, herbicide, pesticide, hormone or plant hormone, plant nutrients, fertilizer, plant protection ingredient, agricultural chemical, agricultural carrier, preservative, flavor or fragrant, nutrients, vitamin, nutrient, color, natural extract, antibody, antibiotics, antimicrobial, food additive or ingredient, supplement additive or ingredients, cosmetic additive or ingredient, additive or ingredient for personal care products, agricultural additive or ingredient, medical additive or ingredients, and industrial products; and
b. said aqueous phase may further contains at least one non-water component selected from a sugar, salt, protein, peptide, flavor, color, vitamin, antioxidant, antimicrobial compound, fragrant, antibody, enzyme, active pharmaceutical ingredient, pharmaceutical excipient, fertilizer, plant hormone, plant nutrient, agricultural chemical, agricultural carrier, herbicide, pesticide, nutrient, food additive and ingredient, cosmetic additive and ingredient, additive or ingredient for personal care products, stabilizer, and emulsifier;

Embodiment 43. The encapsulation composition in embodiment 40, is further processed to a product including but not limited to a food, beverage, fragrance, pharmaceutical product for human or animal consumption, feed product, product for agricultural industry, cosmetic product, construction material and product, industrial cleaning ingredient and product, paint, coating, product for personal hygiene, lotion, shampoo, conditioner, soap, and the like, or the ingredients or combinations of any of above products.

Embodiment 44. The encapsulation composition in embodiment 43 wherein said product is a oleoresin, essential oil, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, cannabis-containing products allowed by law, animal feed, feed additive, pet food, fish feed, fragrance, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, fabric or leather treatment chemical, plastic, rubber, container, utensil, packaging, tire, cloth, fabric, leather, or an ingredient or combinations of any of above products.

Embodiment 45. A product containing the emulsifier of any of embodiments 1-12, or 31 is a food, beverage, dietary supplement, personal care, cosmetics, recreational, smoke, inhaled, pharmaceutical, agricultural, or industrial product

Embodiment 46. The product in embodiment 45, includes but is not limited to an oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, marijuana or cannabis products allowed by law, animal feed, feed additive, pet food, fish feed, fragrance, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, fabric or leather treatment chemical, plastic, rubber, container, utensil, packaging, tire, cloth, fabric, leather and the like, or the ingredient of, or the combination of any of above products.

Embodiment 47. The product of embodiment 46 further contains one or more other food, beverage, dietary supplement, personal care, cosmetics, recreational, smoke, inhaled, pharmaceutical, agricultural, or industrial product ingredients.

Embodiment 48. A composition that comprises the emulsifier of any of claim 1-12, or 31, and a second component.

Embodiment 49. The composition of embodiment 48, wherein the second component comprises one or a plurality of emulsifiers including but not limited to small-molecule emulsifiers, mono/di-glycerides, polysorbates, calcium stearoyl lactylate, sodium stearoyl lactylate, polyglycerol ester, sorbitan ester, propylene glycol ester, sugar ester, acetylated monoglyceride, lactylated monoglycerides, lecithin, saponin, modified starch, gum arabic (gum acacia), protein-based emulsifiers such as pea protein, sodium caseinate, whey protein isolates, or the like.

Embodiment 50. The composition of embodiment 48, wherein the second component comprises one or a plurality of bulking agents including but not limited to starch, maltodextrins, syrups, sugars, sugar alcohols, oligosaccharides, hydrolyzed biopolymers, polysaccharides hydrolysates, protein hydrolysates, or the like.

Embodiment 51. The composition of embodiment 48, wherein the second component comprises one or a plurality of rheological property modifiers including but not limited to polysaccharide gums, proteins, xanthan gum, locus bean gum, guar gum, alginate, pectin, cellulose, carboxymethylcellulose, modified cellulose, starch or its derivatives, protein-based hydrocolloids, gelatin, soy protein, pea protein, egg white, or the like.

Embodiment 52. The composition of embodiment 48, wherein the second component comprises one or a plurality of protein materials including but not limited to pea proteins, soy proteins, cricket powder, protein hydrolysates, whole egg, egg yolk, egg whites, pea flour, bean flours, lentil flour, and the like.

Embodiment 53. The composition of embodiment 48, wherein the second component comprises one or a plurality of components related to prebiotics, probiotics, microbiome-related materials, and the like.

1. An emulsifier composition originated from a plant material comprising:
1 wt % to 85 wt % protein; and
15 wt % to 99 wt % carbohydrate, wherein said carbohydrate includes a starch component, wherein the crystallinity of said starch component in said emulsifier composition is reduced from the crystallinity of the starch component in its native form in the original plant material of the same plant origin,
wherein said emulsifier composition is useful for forming an emulsion of lipophilic material with an emulsifier composition-to-lipophilic material ratio of between 1/100 and 100/1, preferentially between 1/10 and 10/1.

2. The emulsifier composition of claim 1, wherein said plant material comprises at least one component from flour, meal, fraction or whole grain of cereal grain, legume, tuber, root, stem, seed, nut of a plant, or the combination thereof.

3. The emulsifier composition of claim 1, wherein the crystallinity of said starch component in said emulsifier composition is about 90% or less of the crystallinity of the starch component in its native form in the original plant material.

4. The emulsifier composition according to claim 1, wherein said emulsifier composition provides protection to said lipophilic material against photo instability, oxidation, chemical instability, volatility, pH instability, temperature instability, or color instability, taste change, flavor change, etc.

5. A process for producing an emulsifier comprising:
treating plant material with at least one step of milling and at least one step of heating, said milling is under conditions including a period of time and a power input, and said heating is under conditions including a period of time and a temperature,
wherein said plant material is at least one component selected from the group consisting of flour, meal, fraction or whole grain of cereal grain, legume, potato, yam, taco, tuber, root, stem, nut, seed, and combinations thereof,
wherein said emulsifier comprises a protein component ranging from about 1 wt % to about 85 wt % of total dry weight, and a carbohydrate component ranging from about 15 wt % to about 99 wt % of total dry weight, and wherein the carbohydrate component contains a starch, and wherein the crystallinity of said starch in said emulsifier is reduced from the crystallinity of said starch in the original plant material of the same plant origin, and wherein said emulsifier is useful for preparing emulsions of lipophilic materials.

6. The process for producing an emulsifier according to claim 5, wherein the crystallinity of said starch in said emulsifier is about 90% or less of the crystallinity of the starch in the original plant material of the same plant origin.

7. The process for producing an emulsifier according to claim 5, wherein said milling process uses at least one form of mill selected from hammer mill, ball mill, jet mill, stone mill, roller mill, stirred mill, stirred ball mill, colloidal mill, attritor, homogenizer, fluidizer, high speed blender, sigma blender, and extruder, and wherein said milling lasts at least 2 minutes.

8. The process for producing an emulsifier according to claim 5, wherein said milling has a power input of not less than 0.1 kilowatts (kw) per kilogram (kg) of said plant material.

9. The process for producing an emulsifier according to claim 5, wherein said milling has a power input of not less than 0.2 kw per kg of said plant material.

10. The process for producing an emulsifier according to claim 5, wherein said milling has a power input of not less than 0.5 kw per kg of said plant material.

11. The process for producing an emulsifier according to claim 5, wherein said heating uses at least one method or facility of heating selected from oven, vacuum oven, ventilated oven, microwave oven, near infrared oven, steaming, hot gas heating, container with jacket for heating, static heating, stirred heating, jet cooking, temperature regulator or controller, dryer, heat tunnel, heat tube, or heat exchanger, and wherein the temperature for said heating is between 40° C. and 300° C., a stepwise gradient thereof, or a combination of different temperatures thereof, and wherein said heating lasts for at least 2 minutes.

12. The process for producing an emulsifier according to claim 5, wherein said milling and said heating overlap or take no particular order.

13. The process for producing an emulsifier according to claim 5, further comprising a step of hydrothermal treatment of said plant material, wherein said hydrothermal treatment is conducted using at least one from cooking, annealing, gelatinizing, steaming, baking, microwaving, fluid bed treatment, granulation, extruding, and homogenizing said plant material.

14. The process for producing an emulsifier according to claim 13, further comprising a step of drying of said plant material after subjecting it to said hydrothermal treatment.

15. The process for producing an emulsifier according to claim 5 in which said plant material is at least one component selected from the group consisting of flour, meal, fraction or whole grain of cereal grains, legume, tuber, root, stem, seed, nut, and combination thereof, comprising at least one step of milling and at least one step of heating of said plant material to afford said emulsifier, wherein:
a. said at least one step of milling lasts for at least 2 minutes;
b. said at least one step of milling has a power input no less than about 0.1 kw per kg of said plant material;
c. said at least one step of heating is conducted at a temperature from about 40° C. to about 300° C., and for a period of about 2 minutes to about 100 hours;
d. said emulsifier contains both protein component and carbohydrate component including a starch, wherein the content of said protein component is 1% to 85% of the said emulsifier, the content of said carbohydrate component is about 15% to about 99% of the said emulsifier, and the crystallinity of said starch in said emulsifier is less than 90% of the crystallinity of the starch in its native form in the original plant material of the same plant origin.

16. An emulsifier prepared according to the process of claim 5.

17. An emulsion comprising:
an emulsifier of claim 1 or claim 16; and
at least one lipophilic materials,
wherein said emulsion comprises an oil phase and an aqueous phase,
a. wherein the oil phase of said emulsion comprises said at least one lipophilic material, and
b. wherein the aqueous phase of said emulsion contains water or a combination of water and at least one non-water component.

18. The emulsion of claim 17, wherein the stability of said emulsion is at least twice the stability of an emulsion comprising the original plant material from the same plant origin instead of comprising said emulsifier, the stability being determined based on a comparison of at least one property selected from the group consisting of creaming, flocculation, aggregation, sedimentation, or precipitation following application of gravity, centrifugation over time, and combinations thereof.

19. The emulsion of claim 17, wherein said lipophilic material includes a material selected from the group consisting of oil or fat from plant, animal, microbial or petroleum sources, essential oils, other emulsifier, active pharmaceutical ingredient, pharmaceutical excipient, biocide, herbicide, pesticide, hormone or plant hormone, plant nutrients, fertilizer, plant protection ingredient, agricultural chemical, agricultural carrier, preservative, flavor or fragrant, nutrients, lipophilic vitamin, nutrient, colorant, natural extract, antibody, antibiotics, antimicrobial, food additive or ingredient, supplement additive or ingredients, cosmetic additive or ingredient, additive or ingredient for personal care products, agricultural additive or ingredient, medical additive or ingredients, industrial products, and combinations thereof.

20. The emulsion of claim 17, wherein said aqueous phase is a solution, suspension, or a mixture of one or more components selected from the group consisting of sugar, salt, protein, peptide, flavor, color, vitamin, antioxidant, antimicrobial compound, fragrant, antibody, enzyme, active pharmaceutical ingredient, pharmaceutical excipient, fertilizer, plant hormone, plant nutrient, agricultural chemical, agricultural carrier, herbicide, pesticide, nutrient, food additive and ingredient, cosmetic additive and ingredient, additive or ingredient for personal care products, stabilizer, emulsifier, and combinations thereof.

21. The emulsion of claim 17, wherein said emulsion is further processed to products, including but not limited to a food, beverage, food ingredient, pharmaceutical product for human or animal consumption, feed product, plant protection, product for agricultural industry, product for personal care, personal hygiene, including lotion, cream, shampoo, conditioner, soap, industrial product, and the like.

22. An encapsulation composition comprising a dehydration of the emulsion of claim 17.

23. The encapsulation composition of claim 22, processed to a product selected from the group consisting of food, beverage, fragrance, pharmaceutical product for human or animal consumption, feed product, product for agricultural industry, cosmetic product, construction material and product, industrial cleaning ingredient and product, paint, coating, product for personal hygiene, lotion, shampoo, conditioner, soap, and the like, or the ingredients and combinations thereof.

24. A product comprising an emulsifier of claim 1 or claim 16, wherein the product is selected from the group consisting of a food, beverage, dietary supplement, personal care, cosmetics, recreational, smoke, inhaled, pharmaceutical, agricultural, or industrial product, including but is not limited to an oleoresin, essential oil, encapsulation, protein shake, smoothie, cake, muffin, donut, tortilla, bread, flat bread, chip, cracker, cookie, pie, bar, pudding, snack food, batter, dough, baked goods, frozen or refrigerated dough, dessert, icing, topping, filling, candy, ice cream, frozen yoghurt, frozen food, frozen dessert, condiment or culinary food, soup, sauce, dressing, gravy, food entry, coffee creamer, dried or liquid color formulation, dried or liquid flavor formulation, dried or liquid nutrients formulation, DHA or EPA formulation, vitamin formulation, micro nutrients additives, nutrition additive, dietary supplement, supplement ingredients, bakery ingredients mix, beverage ingredients mix, meat product, plant meat alternative products, brine, powder food, dairy, milk alternative, protein drink, energy drink, beverage, soy milk, almond milk, other nuts milk, probiotic or prebiotic drink, yoghurt, cheese, meal replacer, plant protein drink, marijuana or cannabis products, animal feed, feed additive, pet food, fish feed, fragrance, cream, lotion, moisturizer, skincare products, cosmetics, powders, foundation, eye shadow, bronzer, makeup, cleanser, serum, sunscreen, shampoo, conditioner, soap, hair product, detergent, dishwasher, wipe, baby powder, ointment, balm, lip products, household spray, fabric spray, fabric coating, pharmaceutical product for human or animal use, drug, antibiotics, anti-infection drug, anti-viral drug, anti-fundal drug, vaccine, steroid, nasal spray, topical cream, topical ointment, product for agricultural use, pesticide, herbicide, biocide, plant protection, plant nutrients, fertilizer, spray, plant hormone, seed protection, seed coating, fungicides, household or industrial cleaning supply, industrial coating, fabric or leather treatment chemical, plastic, rubber, container, utensil, packaging, tire, cloth, fabric, leather and the like, or the ingredient of, and combinations thereof.

25. The product of claim 24 further containing one or more other ingredients selected from the group consisting of: emulsifiers including but not limited to small-molecule emulsifiers, mono/di-glycerides, polysorbates, calcium stearoyl lactylate, sodium stearoyl lactylate, polyglycerol ester, sorbitan ester, propylene glycol ester, sugar ester, acetylated monoglyceride, lactylated monoglycerides, lecithin, saponin, modified starch, gum arabic (gum acacia), protein-based emulsifiers such as pea protein, sodium caseinate, whey protein isolates, or the like, bulking agents including but not limited to starch, maltodextrins, syrups, sugars, sugar alcohols, oligosaccharides, hydrolyzed biopolymers, polysaccharides hydrolysates, protein hydrolysates, or the like, rheological property modifiers including but not limited to polysaccharide gums, proteins, xanthan gum, locus bean gum, guar gum, alginate, pectin, cellulose, carboxymethylcellulose, modified cellulose, starch or its derivatives, protein-based hydrocolloids, gelatin, soy protein, pea protein, egg white, or the like, protein materials including but not limited to pea proteins, soy proteins, cricket powder, protein hydrolysates, whole egg, egg yolk, egg whites, pea flour, bean flours, lentil flour, and the like, components related to prebiotics, probiotics, microbiome, marijuana, cannabis related materials, or the like, or other ingredients for food, food, beverage, dietary supplement, personal care, cosmetics, recreational, smoke, inhaled, pharmaceutical, agricultural, industrial product, and combinations thereof.

26. The product according to claim 24, wherein said emulsifier provides for the product one or more properties selected from the group consisting of emulsifying, thickening, texture improving, creaminess, improved mouthfeel, improved freeze-thaw stability, improved physical stability, and combinations thereof.
